(19) United States
(12) Patent Application Publication
(10) Pub. No.: US 2021/0269549 A1
(43) Pub. Date: Sep. 2, 2021

(54) ANTI-SAS1B ANTIBODIES, ASSOCIATED METHODS OF USE, AND COMPOSITIONS AND METHODS FOR DETECTING AND TREATING CANCER

(71) Applicant: UNIVERSITY OF VIRGINIA PATENT FOUNDATION, Charlottesville, VA (US)

(72) Inventors: John C. HERR, Charlottesville, VA (US); Eusebio PIRES, Charlottesville, VA (US); Ryan D'SOUZA, Charlottesville, VA (US); Arabinda MANDAL, Charlottesville, VA (US); Kiley ANDERSON-KNAPP, Charlottesville, VA (US)

(73) Assignee: UNIVERSITY OF VIRGINIA PATENT FOUNDATION, Charlottesville, VA (US)

(21) Appl. No.: 17/136,788

(22) Filed: Dec. 29, 2020

Related U.S. Application Data
(63) Continuation of application No. 15/766,258, filed on Apr. 5, 2018, now abandoned.
(60) Provisional application No. 62/237,211, filed on Oct. 5, 2015.
(60) Provisional application No. 62/298,944, filed on Feb. 23, 2016.
(60) Provisional application No. 62/358,453, filed on Jul. 5, 2016.

(51) Int. Cl. C07K 16/30; G01N 33/574; G01N 33/573; A61K 39/295; C07K 16/28; A61K 47/68; A61P 35/00; C07K 2/00; C07K 16/46

(52) U.S. Cl. CPC C07K 16/3069; G01N 33/57492; G01N 33/573; A61K 39/295; C07K 16/28; C07K 16/30; A61K 2039/505; A61P 35/00; C07K 2/00; C07K 16/464; G01N 2333/96419; C07K 2317/55; A61K 47/6869

(57) ABSTRACT

The present disclosure provides anti-SAS1B antibodies, antigen-binding fragments thereof, and antibody-drug conjugates and methods of their use. This present disclosure also provides an isolated antibody or antigen-binding portion thereof having at least one of SB antibodies binding to surface exposed SAS1B, and 6B1 antibodies identifying at least one set of cancer patients testing positive for SAS1B expression.

### CROSS REFERENCE TO RELATED APPLICATIONS

This application is a continuation of U.S. patent application Ser. No. 15/766,258, filed Apr. 5, 2018, which claims the benefit of priority to International Application PCT/US2016/055557, filed Oct. 5, 2016, which is claims priority pursuant to 35 U.S.C. § 119 to U.S. Provisional Application Ser. No. 62/237,211, filed Oct. 5, 2015, Provisional Application Ser. No. 62/298,944, filed Feb. 23, 2016, and Provisional Application Ser. No. 62/358,453, filed Jul. 5, 2016, which are hereby incorporated by reference in their entireties.

### SEQUENCE LISTING

This document incorporates by reference herein an electronic sequence listing text file, which is filed in electronic format via EFS-Web. The text file is named “1548661.txt,” is 20,480 bytes, and was created on Feb. 23, 2016.

### FIELD

The present disclosure relates to anti-SAS1B antibodies, associated methods of use, and compositions and methods for detecting and treating cancer.

### BACKGROUND INFORMATION

Methods are needed in the art to identify and inhibit the growth of cells expressing SAS1B.

### SUMMARY

An isolated antibody or antigen-binding portion thereof comprising a VH CDR1 of SEQ ID NO:1; a VH CDR2 of SEQ ID NO:2 or 6; a VH CDR3 of SEQ ID NO:3, 7, or 10; a VL CDR1 of SEQ ID NO:4, 8, or 11; a VL CDR2 of GAS or KVS; and a VL CDR3 of SEQ ID NO:5 or 9 or 95% identity thereto. An isolated antibody or antigen-binding portion thereof, wherein the antibody comprises (a) a VH CDR1 of SEQ ID NO:1, a VH CDR2 of SEQ ID NO:2, a VH CDR3 of SEQ ID NO:3, a VL CDR1 of SEQ ID NO:4, a VL CDR2 of GAS, a VL CDR3 of SEQ ID NO:5; (b) a VH CDR1 of SEQ ID NO:1, a VH CDR2 of SEQ ID NO:6, a VH CDR3 of SEQ ID NO:7, a VL CDR1 of SEQ ID NO:8, a VL CDR2 of KVS, a VL CDR3 of SEQ ID NO:9; or (c) a VH CDR1 of SEQ ID NO:1, a VH CDR2 of SEQ ID NO:2, a VH CDR3 of SEQ ID NO:10, a VL CDR1 of SEQ ID NO:11, a VL CDR2 of KVS, a VL CDR3 of SEQ ID NO:9.

An isolated antibody or antigen-binding portion thereof that specifically binds human SAS1B, wherein said antibody binds the same human SAS1B epitope recognized by the monoclonal antibody produced by the hybridoma cell line having ATCC number CRL-1581 (Sp2/0-Ag14). An isolated antibody or antigen-binding portion thereof, wherein said antibody or antigen-binding portion specifically binds human SAS1B, wherein the antibody or antigen-binding portion thereof competes for binding with an antibody or antigen-binding portion thereof described herein. An isolated antibody or antigen-binding portion thereof, wherein said antibody or antigen-binding portion thereof inhibits binding of an isolated antibody or antigen-binding portion thereof described herein to human SAS1B.

An isolated antibody or antigen-binding portion thereof, wherein said antibody or antigen-binding portion thereof specifically binds to a polypeptide consisting of amino acids 24-163 of SEQ ID NO:26. An isolated antibody or antigen-binding portion thereof, wherein the antibody or antigen-binding portion thereof is a monoclonal antibody, a chimeric antibody, a humanized antibody, a synthetic antibody, a single chain antibody, a diabody, or a CDR-grafted antibody. An isolated antibody or antigen-binding portion thereof, wherein the antibody or antigen-binding portion thereof comprises a VL amino acid sequence of SEQ ID NOs:13, 15, 17, or 18. An isolated antibody or antigen-binding portion thereof, wherein said antibody or antigen-binding portion thereof comprises the VH amino acid sequence of SEQ ID NOs:12, 14, or 16.

A composition comprising (a) an antibody or antigen-binding portion thereof and a pharmaceutically acceptable carrier; or (b) an antibody or antigen-binding portion thereof, wherein the antibody or antigen-binding portion thereof is conjugated to a therapeutic agent, and a pharmaceutically acceptable carrier. An antibody-drug conjugate (ADC) comprising an antibody or antigen-binding portion thereof, wherein the antibody or antigen-binding portion is conjugated to a therapeutic agent.

An isolated antibody or antigen-binding portion thereof, wherein the antibody or antigen-binding portion thereof specifically binds human SAS1B with an affinity (K_{d}) of at least about 10⁻⁶ M. An isolated antibody or antigen-binding portion thereof, wherein said antibody or antigen-binding portion thereof binds to cancer cells. An isolated cell of hybridoma ATCC number CRL-1581 (Sp2/0-Ag14). An isolated polypeptide consisting of one of SEQ ID NO:1, 2, 3, 4, 5, 6, 7, 8, 9, 10, 11, 12, 13, 14, 15, 16, 17, 18, or amino acids 24-163 of SEQ ID NO:26. An isolated polynucleotide encoding a polypeptide described herein.

An isolated polynucleotide encoding an anti-human SAS1B antibody or antigen-binding portion thereof, wherein said isolated polynucleotide encodes a heavy chain and a light chain, wherein the immunoglobulin heavy chain complementarity determining region (CDR) CDR1 comprises SEQ ID NO:1, CDR2 comprises SEQ ID NO:2 or 6, and CDR3 comprises SEQ ID NO:3, 7, or 10, and wherein the immunoglobulin light chain CDR1 comprises SEQ ID NO:4, 8, or 11, CDR2 comprises GAS or KVS, and CDR3 comprise SEQ ID NO:5 or 9. A vector comprising one or more polynucleotides described herein. A host cell comprising a vector described herein.

A method for producing a human SAS1B antibody or antigen-binding portion thereof, comprising culturing an isolated host cell and recovering said antibody. An isolated antibody or antigen-binding portion thereof, wherein the antibody is a chimeric antibody comprising VL and VH domains obtained from a mouse antibody, wherein said VL and VH domains comprise sequences capable of binding to human SAS1B, and the VL and VH domains are fused to human CL and CH domains, respectively.

A method of treating a hyperproliferative disorder comprising administering a composition described herein to a mammal in need thereof. A method of detecting a SASB1 polypeptide in a sample comprising (a) contacting one or more antibodies with a test sample under conditions that allow polypeptide/antibody complexes to form; and (b) detecting polypeptide/antibody complexes; wherein the detection of polypeptide/antibody complexes is an indication that the human SAS1B polypeptide is present in the sample.

A method of detecting SAS1B-positive cells in a test sample comprising (a) contacting one or more antibodies with the test sample under conditions that allow SAS1B-positive cell/antibody complexes to form; and (b) detecting SAS1B positive cell/antibody complexes; wherein the detection of SAS1B positive cell/antibody complexes is an indication that SAS1B cells are present in the test sample. A method of detecting SAS1B-positive cells in a test sample wherein the sample is lymph node or tissue aspirate, serum, whole blood, cellular suspension, lymphocytes, whole blood, plasma, circulating tumor cells, tumor cells or tissue, ascites fluid, urine, or fluid effusion.

A method of detecting SAS1B-positive cells in a test sample, the method comprising (a) determining the topology of an SAS1B molecule at SAS1B-positive cell surfaces by defining alternative splice variants that function as integral membrane proteins; and (b) mapping surface accessible epitopes recognized by an isolated antibody or antigen-binding portion thereof. A method of detecting SAS1B-positive cells in a test sample, wherein an isolated antibody or antigen-binding portion is a therapeutic monoclonal antibody-drug conjugate (ADC) and/or T-cell immunotherapy that targets SAS1B-positive cell surface epitopes of a SAS1B metalloprotease.

A method of detecting SAS1B-positive cells in a test sample, wherein alternative splice variants are human astacin-like (ASTL) gene splice variants encoding at least one SAS1B protein isoform to traffic at least one site of the SAS1B-positive cell. A method of detecting SAS1B-positive cells in a test sample, wherein at least one site is a plasma membrane. A method of detecting SAS1B-positive cells in a test sample, comprising identifying at least one SAS1B protein isoform by cloning cDNA encoding splice variants into reporter constructs to express a chimeric protein with a mCherry tag at the C-terminus.

A method of detecting SAS1B-positive cells in a test sample, wherein an isolated antibody or antigen-binding portion thereof comprises a VH CDR1 of SEQ ID NO:1; a VH CDR2 of SEQ ID NO:2 or 6; a VH CDR3 of SEQ ID NO:3, 7, or 10; a VL CDR1 of SEQ ID NO:4, 8, or 11; a VL CDR2 of GAS or KVS; and a VL CDR3 of SEQ ID NO:5 or 9 or 95% identity thereto.

A method of detecting SAS1B-positive cells in a test sample, wherein an isolated antibody or antigen-binding portion thereof comprises at least one of (a) SB antibodies binding to surface exposed SAS1B, and (b) 6B1 antibodies identifying at least one set of cancer patients testing positive for SAS1B expression.

The disclosure provides antibodies and antigen-binding portions thereof that specifically bind to the human metallo-endoprotease SAS1B, a product of the ASTL gene. These antibodies and antigen-binding portions thereof have applications in diagnostic assays to measure SAS1B. They also have applications as therapeutic probes, both alone, as “naked” unconjugated antibodies, and conjugated with cytotoxic drugs and radionuclides. These antibodies also have therapeutic use as imaging agents.

Immunochemical, biochemical, morphological, molecular biology, and pharmacological working examples demonstrate several points. Antibodies and antigen-binding fragments have been identified that are specific for the N terminus regions of human SAS1B. These antibodies selectively recognize SAS1B proteins in the presence of an array of other proteins. These antibodies can be used to precipitate SAS1B from extracts of cells that express the SAS1B protein, can bind with SAS1B in fixed and permeabilized cells, and can bind with SAS1B on the surface of SAS1B^{pos} tumor cells. Several of these monoclonal antibodies kill SAS1B^{pos} cancer cells in culture. This killing effect occurs at sub-nanomolar levels of antibody.

In addition antibodies have been screened and selected for their ability to bind to human SAS1B in sections of formalin fixed paraffin embedded tumor tissue as well as normal ovary. These antibodies can be useful for studies of SAS1B on tumor sections using immunohistochemical methods. Furthermore, multiple splice variants of SAS1B exist in both normal ovary and in human tumors.

### BRIEF DESCRIPTION OF THE DRAWINGS

Other aspects and advantages disclosed herein will be described with reference to the following detailed descriptions of exemplary embodiments, when read in conjunction with the accompanying drawings, wherein:

FIG. 1 shows a diagram of the human SAS1B polypeptide;

FIGS. 2A-B shows the cytotoxic effect of anti-human SAS1B mAb SB2 and SB5 on human uterine cancer cell MMMT539 by antibody drug conjugate cytotoxicity (ADC) assay; FIG. 2A shows the percentage of cell viability. FIG. 2B shows the luminescence (RLU);

FIGS. 3A-B shows the effect of anti-EGFR mAb, staurosporine, and an anti-CABYR mAb on uterine cancer cells;

FIG. 4 shows a diagram of a capture assay by sandwich ELISA;

FIG. 5 shows the detection of magnetic bead coupled SB2 and SB5 monoclonal antibodies;

FIG. 6 shows the sensitivity of HRP-coupled monoclonal antibodies to SAS1B;

FIGS. 7A-B shows the cytotoxic profile of SB2, SB4, SB5, staurosporine, and DMSO with pancreatic cancer cells;

FIGS. 8A-B shows the cytotoxic profile of anti-EGFR antibodies, SB2, and anti-CABYR antibodies on renal carcinoma cells;

FIGS. 9A-B shows the cytotoxic profile of SB1, SB3, SB4, and anti-CABYR antibodies on uterine carcinoma cells;

FIG. 10A-B shows the cytotoxic profile of SB1, SB2, SB3, SB5, and SB5 monoclonal antibodies complexed with isotype specific Fab without DMDM;

FIGS. 11A-B shows the cytotoxic profile of SB2, SB4, SB5, staurosporine, and DMSO with pancreatic cancer cells;

FIG. 12A (right panel) shows mouse oocytes stained dark red-brown with an antibody to SAS1B; FIG. 12B (left panel) shows staining with pre-immune sera;

FIG. 13 shows the exon-intron arrangements in splice variants A-F (SV-A-SV-F);

FIG. 14 shows the sequence, domain structure, homology model, and relationship to plasma membrane of splice variant C;

FIG. 15 shows exon-intron splice junctions spanned by primers and amplimer characteristics;

FIG. 16 shows PCR amplification of a 310 bp pan-ASTL amplimer using ASTL specific c-terminus primers from samples of normal human ovarian cDNA;

FIG. 17 shows samples harvested from the mesenteries of PCR assays for the 310 bp amplimer in cases of advanced, disseminated serous ovarian cancer;

FIG. 18 shows amplification of SV-A and SV-C in the uterine cancer MMMT line SNU-539, primary tumor specimens, and xenografts;

FIG. 19 shows a qRT-PCR of an exon 5-6 product for a standard curve produced by a 4-fold dilution series of synthetic ASTL RNA (∘) run with an RNA sample from a SNU-539 uterine cancer cell line (X);

FIG. 20 shows 19 kDa SAS1B C-term extracellular domain purified from HEK-293 cells;

FIG. 21 shows bacterial expression constructs for SV-A and truncated SV-A;

FIG. 22 shows Western blots loaded with recombinant SAS1B SV-A (aa 1-431), SV-C (aa 1-436), mCherry-SAS1B fusion protein, and C-terminus extracellular domain (aa 160-431);

FIG. 23 shows mAbs SB2-5 reacting with endogenous 44 kDa SAS1B extracted from uterine cancer cells;

FIG. 24 shows a confocal image of one mAb, SB4, recognizing native SAS1B in fixed cells transfected with V-5 tagged SAS1B;

FIG. 25 shows a confocal immunofluorescence microscopy image of SB2 monoclonal binding SAS1B on live cells;

FIG. 26 shows staining for SAS1B protein on a paraffin section of macaque ovary using IgG1κ mAb 6B1 [1:16,000 dilution];

FIG. 27 shows regions of SAS1B to which leading mouse and rabbit mAbs have been mapped;

FIG. 28 shows several mAbs in SB series, directed to the N-terminus, as antibody-duocarmycin conjugates in killing uterine tumor cells in vitro;

FIG. 29A shows internal Golgi pool of SAS1B SV-A in Cos7 cells co-localized with SAS1B-C-term mCherry signal after cell permeabilization with TX-110; FIG. 29B shows the surface pool of SAS1B SV-C with no permeabilization;

FIGS. 30A and 30B show application of reporter strategy in studies of SAS1B internalization; FIG. 30A shows no staining with mCherry antibody, SAS1B antibody, and endosomal marker EEA1 in Cos-7 cells transfected with the empty vector; and FIG. 30B shows SAS1B antibody complex endocytosed and merged with EEA1 vesicles in cells transfected with the SV-C isoform of SAS1B.

FIGS. 31-47D disclose data and other information supporting aspects of the embodiments already described with respect to FIGS. 12A-30B.

### DETAILED DESCRIPTION

As used herein, the singular forms “a,” “an,” and “the” include plural referents unless the context clearly dictates otherwise. The term “about” in association with a numerical value means that the numerical value can vary plus or minus by 5% or less of the numerical value.

In the following detailed description, numerous specific embodiments are set forth in order to provide a thorough understanding of the compositions and methods disclosed herein. However, as will be apparent to those skilled in the art, the present embodiments may be practiced without these specific details or by using alternate elements or processes. In other instances, well-known processes, procedures, and/or components have not been described in detail so as not to unnecessarily obscure aspects of embodiments disclosed herein.

Features regarding exemplary isolated antibodies or antigen-binding portions thereof are described followed by features relating to compositions and methods for detecting and treating cancer.

### Isolated Antibodies or Antigen-Binding Portions Thereof

### Polypeptides

A polypeptide is a polymer of three or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure.

A light or heavy chain variable region of an antibody has four framework regions interrupted by three hypervariable regions, known as complementary determining regions (CDRs). CDRs determine the specificity of antigen binding. The heavy chain and light chain each have three CDRs, designated from the N terminus as CDR1, CDR2, and CDR3 with the four framework regions flanking these CDRs. The amino acid sequences of the framework region are highly conserved and CDRs can be transplanted into other antibodies. Therefore, a recombinant antibody can be produced by combining CDRs from one or more antibodies with the framework of one or more other antibodies. Antibodies of the disclosure include antibodies that comprise at least one, two, three, four, five, or six (or combinations thereof) of the CDRs of any of the monoclonal antibodies isolated from the hybridomas shown in Table 1, or variant CDRs. Variant CDRs are CDRs comprising amino acid sequences similar to the amino acid sequences of CDRs of any of the monoclonal antibodies produced by the hybridomas shown in Table 1. In one embodiment of the disclosure variant CDRs specifically bind to amino acids 24-163 of SEQ ID NO:26 when present in an appropriate antibody structure (e.g., framework regions and other appropriate CDRs).

Polypeptides of the present disclosure comprise full-length human, mouse, or rabbit anti-SAS1B heavy chain variable regions, full-length human, mouse or rabbit light chain regions, fragments thereof, and combinations thereof.

TABLE 1

| | | Specifically binds | Specifically binds |
|---|---|---|---|
| | | mouse and human | human SAS1B (and |
| | Isotype | SAS1B | not mouse SAS1B) |
| SB1 | IgG2a | Yes | |
| SB2 | IgG2b | | Yes |
| SB3 | IgG2b | | Yes |
| SB4 | IgG2a | | Yes |
| SB5 | IgG2b | | Yes |
| SB6 | IgG2b | | Yes |
| SB7 | IgG2b | | Yes |

TABLE 2

| | Heavy Variable | Light Variable |
|---|---|---|
| | Chain Amino acid | Chain Amino acid |
| SB1 | SEQ ID NO: 12 | SEQ ID NO: 13 |
| SB2 | SEQ ID NO: 14 | SEQ ID NO: 15 |
| SB3 | SEQ ID NO: 14 | SEQ ID NO: 15 |
| SB4 | SEQ ID NO: 16 | SEQ ID NO: 17 |
| SB5 | SEQ ID NO: 16 | SEQ ID NO: 18 |
| SB6 | SEQ ID NO: 16 | SEQ ID NO: 18 |
| SB7 | SEQ ID NO: 16 | SEQ ID NO: 18 |

TABLE 3

| | Heavy Variable | Light Variable |
|---|---|---|
| | Chain Nucleic acid | Chain Nucleic acid |
| SB1 | SEQ ID NO: 19 | SEQ ID NO: 20 |
| SB2 | SEQ ID NO: 21 | SEQ ID NO: 22 |
| SB3 | SEQ ID NO: 21 | SEQ ID NO: 22 |
| SB4 | SEQ ID NO: 23 | SEQ ID NO: 24 |
| SB5 | SEQ ID NO: 23 | SEQ ID NO: 25 |
| SB6 | SEQ ID NO: 23 | SEQ ID NO: 25 |
| SB7 | SEQ ID NO: 23 | SEQ ID NO: 25 |

TABLE 4

| | CDR1 | CDR2 | CDR3 | CDR1 | CDR2 | CDR3 |
|---|---|---|---|---|---|---|
| | HEAVY | HEAVY | HEAVY | LIGHT | LIGHT | LIGHT |
| | VARIABLE | VARIABLE | VARIABLE | VARIABLE | VARIABLE | VARIABLE |
| SB1 | GYTFTDYN | INPNNGGT | ATNEY | ENVGTY | GAS | GQSYSYPWT |
| | SEQ ID | SEQ ID | SEQ ID NO: 3 | SEQ ID NO: 4 | | SEQ ID NO: 5 |
| | NO: 1 | NO: 2 | | | | |
| SB2 | GYTFTDYN | VNPNNGGT | VPNWDWFAY | QSLVHSNGNTY | KVS | FQGSHVPFT |
| | SEQ ID | SEQ ID | SEQ ID NO: 7 | SEQ ID NO: 8 | | SEQ ID NO: 9 |
| | NO: 1 | NO: 6 | | | | |
| SB3 | GYTFTDYN | VNPNNGGT | VPNWDWFAY | QSLVHSNGNTY | KVS | FQGSHVPFT |
| | SEQ ID | SEQ ID | SEQ ID NO: 7 | SEQ ID NO: 8 | | SEQ ID NO: 9 |
| | NO: 1 | NO: 6 | | | | |
| SB4 | GYTFTDYN | INPNNGGT | APNWDWFAY | QSILHSNGNTY | KVS | FQGSHVPFT |
| | SEQ ID | SEQ ID | SEQ ID NO: 10 | SEQ ID NO: 11 | | SEQ ID NO: 9 |
| | NO: 1 | NO: 2 | | | | |
| SB5 | GYTFTDYN | INPNNGGT | APNWDWFAY | QSILHSNGNTY | KVS | FQGSHVPFT |
| | SEQ ID | SEQ ID | SEQ ID NO: 10 | SEQ ID NO: 11 | | SEQ ID NO: 9 |
| | NO: 1 | NO: 2 | | | | |
| SB6 | GYTFTDYN | INPNNGGT | APNWDWFAY | QSILHSNGNTY | KVS | FQGSHVPFT |
| | SEQ ID | SEQ ID | SEQ ID NO: 10 | SEQ ID NO: 11 | | SEQ ID NO: 9 |
| | NO: 1 | NO: 2 | | | | |
| SB7 | GYTFTDYN | INPNNGGT | APNWDWFAY | QSILHSNGNTY | KVS | FQGSHVPFT |
| | SEQ ID | SEQ ID | SEQ ID NO: 10 | SEQ ID NO: 11 | | SEQ ID NO: 9 |
| | NO: 1 | NO: 2 | | | | |

An antibody of the present disclosure can comprise a VH (variable heavy chain) of SEQ ID NOs:12, 14, or 16. An antibody of the present disclosure can comprise a VL (variable light chain) of SEQ ID NOs:13, 15, 17, or 18. An antibody can comprise a VH CDR1 of SEQ ID NO:1. An antibody of the present disclosure can comprise a VH CDR 2 of SEQ ID NOs:2 or 6. An antibody of the present disclosure can comprise a VH CDR 3 of SEQ ID NOs:3, 7, or 10. An antibody of the present disclosure can comprise a VL CDR 1 of SEQ ID NOs:4, 8, or 11. An antibody of the present disclosure can comprise a VL CDR 2 of GAS or KVS. An antibody of the present disclosure can comprise a VL CDR3 of SEQ ID NO:5 or 9.

An antibody of the present disclosure can comprise a VH nucleic acid sequence of SEQ ID NO:19, 21, or 23. An antibody of the present disclosure can comprise a VL nucleic acid sequence of SEQ ID NO:20, 22, 24, OR 25.

An antibody of the present disclosure can comprise a VH of SEQ ID NO:12 and a VL of SEQ ID NO:13. An antibody of the present disclosure can comprise a VH of SEQ ID NO:14 and a VL of SEQ ID NO:15. An antibody of the present disclosure can comprise a VH of SEQ ID NO:16 and a VL of SEQ ID NO:17. An antibody of the present disclosure can comprise a VH of SEQ ID NO:16 and a VL of SEQ ID NO:18.

An antibody of the present disclosure can comprise a VH CDR1 of SEQ ID NO:1, a VH CDR2 of SEQ ID NO:2; and a VH CDR3 of SEQ ID NO:3. An antibody of the present disclosure can comprise a VL CDR1 of SEQ ID NO:4, a VL CDR2 of GAS; and a VL CDR3 of SEQ ID NO:5. An antibody of the present disclosure can comprise a VH CDR1 of SEQ ID NO:1, a VH CDR2 of SEQ ID NO:6; and a VH CDR3 of SEQ ID NO:7. An antibody of the present disclosure can comprise a VL CDR1 of SEQ ID NO:8, a VL CDR2 of KVS; and a VL CDR3 of SEQ ID NO:9. An antibody of the present disclosure can comprise a VH CDR1 of SEQ ID NO:1, a VH CDR2 of SEQ ID NO:2, a VH CDR3 of SEQ ID NO:10. An antibody of the present disclosure can comprise a VL CDR1 of SEQ ID NO:11, a VL CDR2 of KVS, a VL CDR3 of SEQ ID NO:9. An antibody of the present disclosure may have any of the above VHs combined with any of the above VLs. An antibody of the present disclosure can have any combination of VH CDR1, VH CDR2, VH CDR3, VL CDR1, VL CDR2, VL CDR3, variant VH CDR1, variant VH CDR2, variant VH CDR3, variant VL CDR1, variant VL CDR2, or variant VL CDR3. In one embodiment of the present disclosure an antibody comprises a VH of SEQ ID NOs:12, 14, or 16 (or a variant thereof) and at least one, two or three VL CDRs of SEQ ID NOs:4, 5, 8, 9, 11, KVS, or GAS (or a variant thereof). In one embodiment of the present disclosure an antibody comprises a VL of SEQ ID NOs:13, 15, 17, or 18 (or a variant thereof) and at least one, two or three VH CDRs of SEQ ID NOs:1, 2, 3, 6, 7, or 10 (or a variant thereof).

An antibody of the present disclosure can comprise the variable heavy chain CDRs from antibody SB1, SB2, SB3, SB4, SB5, SB6 or SB7. An antibody of the present disclosure can comprise the variable light chain CDRs from antibody SB1, SB2, SB3, SB4, SB5, SB6 or SB7. An antibody of the present disclosure can comprise a variable light chain that comprises the amino acid sequence of at least one or at least two or at least 3 CDRs of the SB1, SB2, SB3, SB4, SB5, SB6 or SB7 antibody variable light chains. An antibody of the present disclosure can comprise a variable heavy chain that comprises the amino acid sequence of at least one or at least two or at least 3 CDRs of the SB1, SB2, SB3, SB4, SB5, SB6 or SB7 antibody variable heavy chains.

Heavy chain CDRs can be combined with appropriate variable regions of an antibody light chain. Light chain CDRs combined with heavy chain CDRs are, for example, CDRs comprising SEQ ID NOs:4, 5, 8, 9, 11, GAS and KVS, or CDRs functionally equivalent to these CDRs. The respective amino acid sequences correspond to CDR1 (SEQ ID NOs:4, 8, and 11), CDR2 (GAS and KVS), and CDR3 (SEQ ID NO:5 and 9) of an antibody light chain. Alternatively, these light chain CDRs may be used independently of the heavy chains described above. The CDRs are substituted for the corresponding CDR1, CDR2, and CDR3, between the framework of a desired light chain variable region.

Light chain CDRs can be combined with appropriate variable regions of an antibody heavy chain. Heavy chain CDRs combined with light chain CDRs are, for example, CDRs comprising SEQ ID NOs:1, 2, 3, 6, 7, and 10, or CDRs functionally equivalent to these CDRs. The respective amino acid sequences correspond to CDR1 (SEQ ID NO:1), CDR2 (SEQ ID NO:2 and 6), and CDR3 (SEQ ID NOs:3, 7, and 10) of an antibody light chain. Alternatively, these heavy chain CDRs may be used independently of the light chains described above. The CDRs are substituted for the corresponding CDR1, CDR2, and CDR3 regions, between the framework of a desired heavy chain variable region.

A polypeptide variant or variant CDR differs by about, for example, 1, 2, 3, 4, 5, 6, 7, 8, 9, 10, 20, 30, 40, 50, 60 or more amino acid residues (e.g., amino acid additions, substitutions or deletions) from a polypeptide shown in SEQ ID NOs:1-18 or a fragment thereof. Where this comparison requires alignment the sequences are aligned for maximum homology. The site of variation can occur anywhere in the polypeptide. In one embodiment of the present disclosure a variant polypeptide has activity substantially similar to a polypeptide shown in SEQ ID NOs:1-18. Activity substantially similar means that when the polypeptide is used to construct an antibody, the antibody has the same or substantially the same activity as an antibody shown in Table 1.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989)). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art.

The variant polypeptides can have conservative amino acid substitutions at one or more predicted non-essential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A variant polypeptide can also be isolated using a hybridization technique. Briefly, DNA having a high homology to the whole or part of a nucleic acid sequence of SEQ ID NOs:19-25 or a nucleic acid molecule encoding a polypeptide shown in SEQ ID NOs:1-18 is used to prepare a polypeptide. Therefore, a polypeptide of the present disclosure also includes polypeptides that are variants of SEQ ID NOs:1-18, and polypeptides that are encoded by a nucleic acid molecule that hybridizes under high stringency with a nucleic acid molecule encoding SEQ ID NOs:19-25, or a complement thereof. One of skill in the art can easily determine nucleic acid sequences that encode polypeptides of the present disclosure using readily available codon tables. As such, these nucleic acid sequences are not presented herein.

As used herein, percent identity of two amino acid sequences (or of two nucleic acid sequences) is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al. (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the present disclosure.

Identity or identical means amino acid sequence (or nucleic acid sequence) similarity and has an art recognized meaning. Sequences with identity share identical or similar amino acids (or nucleic acids). Sequence identity is the percentage of amino acids identical to those in the antibody's original amino acid sequence, determined after the sequences are aligned and gaps are appropriately introduced to maximize the sequence identity as necessary. Thus, a candidate sequence sharing 85% amino acid sequence identity with a reference sequence requires that, following alignment of the candidate sequence with the reference sequence, 85% of the amino acids in the candidate sequence are identical to the corresponding amino acids in the reference sequence, and/or constitute conservative amino acid changes.

Antibodies of the present disclosure can comprise CDRs of SB1, SB2, SB3, SB4, SB5, SB6 or SB7 antibodies or variant antibodies comprising one or more variant CDRs. These variant antibodies can have an activity equivalent (e.g., binding to human SAS1B with the same or substantially similar K_{d} as an antibody produced by a hybridoma of Table 1) to that of SB1, SB2, SB3, SB4, SB5, SB6 or SB7. Antibody variants retain substantially the same functional activity of SB1, SB2, SB3, SB4, SB5, SB6 or SB7 antibodies. Naturally-occurring functionally active variant antibodies such as allelic variants and species variants and non-naturally occurring functionally active variants are included in the present disclosure and can be produced by, for example, mutagenesis techniques or by direct synthesis. Antibody variants are encoded by variant polypeptides and variant CDRs of SEQ ID NOs:1-18.

The present disclosure also includes polypeptide variants or CDR variants of SEQ ID NOs:1-18. Polypeptide variants or CDR variants of SEQ ID NOs:1-18 can comprise one or more amino acid substitutions, additions or deletions. In one embodiment, a variant polypeptide or variant CDR includes an amino acid sequence at least about 75% identical to a sequence shown as SEQ ID NOs:1-18. In one embodiment, the variant polypeptide or CDR is at least about 75%, 80%, 85%, 90%, 95%, 98%, 99%, 99.5% or more identical to SEQ ID NOs:1-18. Variant polypeptides or variant CDRs encode a variant antibody, which is an antibody comprising an amino acid sequence of SEQ ID NOs:1-18 in which one or more amino acid residues have been added, substituted or deleted. For example, the variable region of an antibody can be modified to improve its biological properties, such as antigen binding. Such modifications can be achieved by e.g., site-directed mutagenesis, PCR-based mutagenesis, cassette mutagenesis. Variant antibodies comprise an amino acid sequence which is at least about 75%, 80%, 85%, 90%, 95%, 98%, 99%, 99.5% or more identical to the amino acid sequence of a heavy or light chain variable region of SB1, SB2, SB3, SB4, SB5, SB6 or SB7. In one embodiment of the present disclosure, a variant antibody retains the same function of a SB1, SB2, SB3, SB4, SB5, SB6 or SB7 antibody (e.g., binds human SAS1B, in particular an N-terminal region of human SAS1B such as amino acids 24-163 of SEQ ID NO:26) at the same or substantially similar K_{d} as an antibody produced by the hybridomas shown in Table 1, e.g. within about 1, 2, 3, 4, 5, 6, 7, 8, 9, 10, 15, or 20% of the K_{d} of an antibody produced by the hybridomas shown in Table 1). In another embodiment of the present disclosure, a variant antibody may have a function that is somewhat altered from a SB1, SB2, SB3, SB4, SB5, SB6 or SB7 antibody (e.g., binding human SAS1B with a K_{d} that is higher or lower than an SB1, SB2, SB3, SB4, SB5, SB6 or SB7 antibody).

Polypeptide sequences can be modified, for example, by synthesizing multiple polynucleotides encoding the amino acid sequence of a variable region, and preparing nucleic acids encoding the variable region by PCR using the polynucleotides. Antibodies that comprise one or more CDRs can be prepared by inserting the polynucleotide into an appropriate expression vector and expressing the polynucleotide. For example, polynucleotides can be synthesized using mixed nucleotides to prepare a DNA library that encodes a variety of antibodies comprising CDRs with various amino acids introduced at certain positions. An antibody can be isolated by selecting from the library a clone encoding an antibody that binds to human SAS1B with a K_{d} that is the same or substantially similar to the K_{d} of an antibody produced by a hybridoma shown in Table 1.

A polypeptide or antibody of the present disclosure can be covalently or non-covalently linked to an amino acid sequence to which the polypeptide or antibody is not normally associated with in nature. Additionally, a polypeptide or antibody of the present disclosure can be covalently or non-covalently linked to compounds or molecules other than amino acids. For example, a polypeptide or antibody can be linked to an indicator reagent, an amino acid spacer, an amino acid linker, a signal sequence, a stop transfer sequence, a transmembrane domain, a protein purification ligand, or a combination thereof. In one embodiment of the present disclosure a protein purification ligand can be one or more C amino acid residues at, for example, the amino terminus or carboxy terminus of a polypeptide of the present disclosure. An amino acid spacer is a sequence of amino acids that are not usually associated with a polypeptide or antibody of the present disclosure in nature. An amino acid spacer can comprise about 1, 5, 10, 20, 100, or 1,000 amino acids.

If desired, a polypeptide can be a fusion protein, which can also contain other amino acid sequences, such as amino acid linkers, amino acid spacers, signal sequences, TMR stop transfer sequences, transmembrane domains, as well as ligands useful in protein purification, such as glutathione-S-transferase, histidine tag, and staphylococcal protein A, or combinations thereof. A fusion protein is two or more different amino acid sequences operably linked to each other. A fusion protein construct can be synthesized chemically using organic compound synthesis techniques by joining individual polypeptide fragments together in fixed sequence. A fusion protein construct can also be expressed by a genetically modified host cell (such as E. coli) cultured in vitro, which carries an introduced expression vector bearing specified recombinant DNA sequences encoding the amino acids residues in proper sequence. The heterologous polypeptide can be fused, for example, to the N-terminus or C-terminus of a polypeptide of the present disclosure. A polypeptide of the present disclosure can also comprise homologous amino acid sequences, i.e., other immunoglobulin-derived sequences. More than one polypeptide of the present disclosure can be present in a fusion protein. Fragments of polypeptides of the present disclosure can be present in a fusion protein of the present disclosure. A fusion protein of the present disclosure can comprise, e.g., one or more of SEQ ID NOs:1-18, fragments thereof, or combinations thereof. Polypeptides of the present disclosure can be in a multimeric form. That is, a polypeptide can comprise two or more copies of SEQ ID NOs:1-18 or a combination thereof.

In one embodiment of the present disclosure, a polypeptide of the present disclosure is derived from a human, rabbit, mouse, other mammal, or combinations thereof. A polypeptide of the present disclosure can be isolated from cells or tissue sources using standard protein purification techniques. Polypeptides of the present disclosure can also be synthesized chemically or produced by recombinant DNA techniques. For example, a polypeptide of the present disclosure can be synthesized using conventional peptide synthesizers.

A polypeptide of the present disclosure can be produced recombinantly. A polynucleotide encoding a polypeptide of the present disclosure can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system.

### Antibodies

The term “antibodies” refers to an intact antibody or an antigen-binding portion or fragment thereof that competes with the intact antibody for antigen binding. The term “antibodies” also includes any type of antibody molecule or specific binding molecule that specifically binds SAS1B. The terms “antigen-binding portion” of an antibody, “antigen-binding fragment” of an antibody, and the like, as used herein, include any naturally occurring, enzymatically obtainable, synthetic, or genetically engineered polypeptide, glycoprotein, or immunoglobulin that specifically binds SAS1B to form a complex.

Antigen-binding fragments of an antibody may be derived, e.g., from full antibody molecules using any suitable standard techniques such as proteolytic digestion or recombinant genetic engineering techniques involving the manipulation and expression of nucleic acids encoding antibody variable and optionally constant domains.

An antibody of the present disclosure can be any isotype including IgG (IgG1, IgG2, IgG2a, Ig2b, IgG3, IgG4), IgM, IgA (IgA1 and IgA2), IgD, and IgE.

A monoclonal antibody is an antibody obtained from a group of substantially homogeneous antibodies. A group of substantially homogeneous antibodies can contain a small amount of mutants or variants. Monoclonal antibodies are highly specific and interact with a single antigenic site. Each monoclonal antibody typically targets a single epitope, while polyclonal antibody populations typically contain various antibodies that target a group of diverse epitopes. Monoclonal antibodies can be produced by many methods including, for example, hybridoma methods (Kohler and Milstein, Nature 256:495, 1975), recombination methods (U.S. Pat. No. 4,816,567), and isolation from phage antibody libraries (Clackson et al., Nature 352:624-628, 1991; Marks et al., J. Mol. Biol. 222:581-597, 1991).

A “humanized antibody or antigen-binding fragment” thereof is an antibody or fragment thereof that has been engineered to comprise one or more human framework regions in the variable region together with non-human (e.g., mouse, rabbit, rat, or hamster) complementarity-determining regions (CDRs) of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations.

A “human antibody or antigen binding fragment thereof” is an antibody or antigen binding fragment thereof that contains only human-derived amino acid sequences. For example, a fully human antibody may be produced from a human B-cell or a human hybridoma cell. In additional embodiments, the antibody may be produced from a transgenic animal that contains the locus for a human heavy chain immunoglobulin and a human light chain immunoglobulin, or contains a nucleic acid that encodes the heavy and light chains of a specific human antibody. A human antibody or antigen binding fragment thereof is still considered a “human antibody or antigen binding fragment thereof” even if the framework and/or CDRs of the heavy chain variable domain or light chain variable domain of the antibody isolated or obtained from a human cell, human cell line, or other methodology are mutated (e.g., by amino acid substitution(s), addition(s), and/or deletion(s)) to improve the affinity or other properties of the antibody. In certain embodiments, after the human antibody isolated or obtained from a human cell or human cell line is mutated so that it has at least about 85%, 86%, 87%, 88%, 89%, 90%, 91%, 92%, 93%, 94%, 95%, 96%, 97%, 98%, 99%, or 99.5% identity to the amino acid sequence of the antibody isolated or obtained from a human cell or human cell line. In some embodiments, six, five, four, three, two, or one amino acid substitutions are made in one, two, three, four, five, and/or six of the CDRs. In some embodiments, six, five, four, three, two, or one amino acid substitutions are made in one, two, three, or four framework regions of the heavy chain variable region, one, two, three, or four framework regions of the light chain variable region of the antibody, Fc, hinge region, or combinations thereof. In one embodiment, a human antibody of the present disclosure has an amino acid sequence that is substantially identical to an antibody isolated or obtained from a human cell or human cell line, but is not naturally occurring. The non-naturally occurring human antibody has one or more mutations in the amino acid sequence that do not occur in the variable heavy or light CDR regions, and do not affect the binding or therapeutic characteristics of the human antibody.

Chimeric antibodies or antigen-binding portions thereof have a part of a heavy chain and/or light chain that is derived from a specific species or a specific antibody class or subclass, and the remaining portion of the chain is derived from another species, or another antibody class or subclass. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397.

Chimeric antibodies can be produced using a variety of techniques including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28:489-498 (1991); Studnicka et al., Protein Engineering 7(6):805-814 (1994); Roguska et al., PNAS 96:969-973 (1994)), and chain shuffling (U.S. Pat. No. 5,565,332).

In one embodiment, a chimeric antibody can comprise variable and constant regions of species that are different from each other, for example, an antibody can comprise the heavy chain and light chain variable regions of a non-human mammal such as a mouse or rabbit, and the heavy chain and light chain constant regions of a human. Such an antibody can be obtained by ligating a polynucleotide encoding a variable region of a mouse or rabbit antibody to a polynucleotide encoding a constant region of a human antibody; incorporating the ligated polynucleotides into an expression vector; and introducing the vector into a host cell for production of the antibody. See WO 96/02576. The host cells can be eukaryotic cells, such as mammalian cells, including, e.g., CHO cells, lymphocytes, and myeloma cells. The chimeric antibody can comprise additional amino acid acids that are not included in the CDRs introduced into the recipient antibody, nor in the framework sequences. These amino acids can be introduced to more accurately optimize the antibody's ability to recognize and bind to an antigen. For example, as necessary, amino acids in the framework region of an antibody variable region can be substituted such that the CDR of a reshaped antibody forms an appropriate antigen-binding site. See Sato et al., Cancer Res. (1993) 53:851-856.

Non-limiting examples of antigen-binding fragments of antibodies include: Fab fragments; Fab′ fragments, Fab′-SH fragments, F(ab′)₂ fragments; Fd fragments; Fv fragments; single-chain Fv (scFv) molecules; sdAb fragments (nanobodies); Fab-like antibodies (an antigen-binding fragment containing variable regions of a heavy chain and light chain that is equivalent to Fab fragments that are obtained by papain digestion); F(ab′)₂-like antibodies (an antigen-binding fragment containing two antigen-binding domains that is equivalent to F(ab′)₂ fragments that are obtained by pepsin digestion), multispecific antibodies prepared from antibody fragments, diabody, bispecific antibody, multifunctional antibody, chimeric antibody, humanized antibody, human antibody, murine antibody, rabbit antibody synthetic antibody, CDR-grafted antibody, and minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g, monovalent nanobodies, bivalent nanobodies), single-chain (Fv)₂ (sc(Fv)₂); divalent (sc(Fv)₂); tetravalent ([sc(Fv)₂]₂) scFV antibodies, and small modular immunopharmaceuticals (SMIPs), and shark variable IgNAR domains, are also encompassed within the expression “antigen-binding fragment,” as used herein.

An antigen-binding fragment of an antibody will typically comprise at least 1 variable domain. The variable domain may be of any size or amino acid composition and will generally comprise at least 1, 2 or 3 CDRs, which are adjacent to or in frame with 1, 2, 3, or 4 framework sequences, in antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

Antigen-binding fragments can be produced by recombinant DNA techniques or by enzymatic or chemical cleavage of intact antibodies.

A “diabody” is a bivalent minibody constructed by gene fusion (see, e.g., Holliger et al., Proc. Natl. Acad. Sci. U.S.A., 90:6444 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between about 2 to 12 residues (e.g., about 2, 3, 4, 5, 6, 7, 8, 9, 10, 11, or 12). The linkers of the polypeptides in a diabody are typically too short to allow the VL and VH to bind to each other. Diabody technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

A scFv is a single-chain polypeptide antibody obtained by linking the VH and VL with a linker (see e.g., Huston et al., PNAS USA, 85:5879 (1988); Pluckthun, “The Pharmacology of Monoclonal Antibodies” Vol. 113, Ed Resenburg & Moore, Springer Verlag, New York, pp. 269-315, (1994)). The order of VHs and VLs to be linked is not particularly limited, and they may be arranged in any order. Examples of arrangements include: VH-linker-VL; or VL-linker-VH. The H chain V region and L chain V region in a scFv may be derived from any anti-SAS1B antibody or antigen-binding fragment thereof described herein.

A sc(Fv)₂ is a fragment where two VHs and two VLs are linked by a linker to form a single chain (Hudson et al., J. Immunol. Methods, 231:177 (1999)). A sc(Fv)₂ molecule can be prepared, for example, by connecting scFvs with a linker. sc(Fv)₂ molecules can include antibodies where two VHs and two VLs are arranged in the order of: VH, VL, VH, and VL (VH-linker-VL-linker-VH-linker-VL), beginning from the N terminus of a single-chain polypeptide; however the order of the two VHs and two VLs is not limited to this arrangement, and they may be arranged in any order. Examples of arrangements are listed below:

### VL-linker-VH-linker-VH-linker-VL;

### VH-linker-VL-linker-VL-linker-VH;

### VH-linker-VH-linker-VL-linker-VL;

### VL-linker-VL-linker-VH-linker-VH; or

### VL-linker-VH-linker-VL-linker-VH.

Three linkers are usually required when four antibody variable regions are linked; the linkers used may be identical or different. There is no limitation on the linkers that link the VH and VL regions of the antibody fragments. In some embodiments, the linker is a peptide linker. Any arbitrary single-chain peptide comprising about three to 25 residues (e.g., 5, 6, 7, 8, 9, 10, 11, 12, 13, 14, 15, 16, 17, 18 amino acids) can be used as a linker. Examples of such peptide linkers include: Ser; Gly Ser; Gly Gly Ser; Ser Gly Gly; Gly Gly Gly Ser (SEQ ID NO:27); Ser Gly Gly Gly (SEQ ID NO:28); Gly Gly Gly Gly Ser (SEQ ID NO:29); Ser Gly Gly Gly Gly (SEQ ID NO:30); Gly Gly Gly Gly Gly Ser (SEQ ID NO:31); Ser Gly Gly Gly Gly Gly (SEQ ID NO:32); Gly Gly Gly Gly Gly Gly Ser (SEQ ID NO:33); Ser Gly Gly Gly Gly Gly Gly (SEQ ID NO:34); (Gly Gly Gly Gly Ser (SEQ ID NO:35)ₙ, wherein n is an integer of one or more; and (Ser Gly Gly Gly Gly (SEQ ID NO:36)ₙ, wherein n is an integer of one or more.

In certain embodiments, the linker is a synthetic compound linker (chemical cross-linking agent). Examples of cross-linking agents include, for example, N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2 (sulfosuccinimidooxycarbonyloxy)ethyl]sulfone (sulfo-BSOCOES).

Bispecific antibodies are antibodies that have binding specificities for at least two different epitopes. Exemplary bispecific antibodies may bind to two different epitopes of the SAS1B protein. Other such antibodies may combine an SAS1B binding site with a binding site for another protein. Bispecific antibodies can be prepared as full length antibodies or low molecular weight forms thereof (e.g., F(ab′)₂ bispecific antibodies, sc(Fv)₂ bispecific antibodies, diabody bispecific antibodies). Full length bispecific antibodies can be produced based on the co-expression of two immunoglobulin heavy chain-light chain pairs, where the two chains have different specificities (Millstein et al., Nature, 305:537-539 (1983)). Alternatively, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields. Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

Antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises an Fc region or a hinge region. A multivalent antibody can comprise about 3, 4, 5, 6, 7, 8, or more antigen binding sites. The multivalent antibody optionally comprises at least one, two, three or more polypeptide chains, wherein the polypeptide chain(s) comprise two or more variable domains. For instance, the polypeptide chain(s) may comprise VDI-(XI)ₙ-VD2-(X2)ₙ-Fc, wherein VD1 is a first variable domain, VD2 is a second variable domain, Fc is a polypeptide chain of an Fc region, XI and X2 represent an amino acid or peptide spacer, and n is 0 or 1.

### Epitopes

One embodiment of the present disclosure provides binding molecules (e.g., antibodies or antigen-binding fragments) that specifically bind to human SAS1B. In one embodiment, the antibodies or antigen-binding fragments thereof specifically bind to an epitope within the N-terminal domain (e.g., amino acids 24-163 of human SAS1B (SEQ ID NO:26). In a specific embodiment, the antibodies or antigen-binding fragments thereof specifically bind to an epitope within amino acids 24-90, 143-163, 24-34, 35-45, 46-56, 57-67, 68-78, 79-89, 90-100, 101-111, 112-122, 123-133, 134-144, 145-146, 147-157, 158-163 of SEQ ID NO:26 or combinations thereof. The antibody or antigen-binding portion thereof may bind to conformational epitope which comprises 2 or more of these regions.

An antibody or fragment thereof of the present disclosure binds to an epitope that overlaps with or is the same (i.e., a substantially identical epitope) as any of the monoclonal antibodies shown in Table 1. An antibody that binds to an epitope substantially identical to an epitope of human SAS1B to which a monoclonal antibody of Table 1 binds, can be obtained by analyzing epitopes of the monoclonal antibodies of Table 1 using well known epitope mapping methods. Competitive assays can be used to determine if two antibodies bind to a substantially identical epitope of SAS1B. Where the binding of a first anti-SAS1B antibody with SAS1B is competitively inhibited by a second anti-SAS1B antibody, the first antibody and the second antibody can be considered to bind to a substantially identical epitope on SAS1B. Competitively inhibits means that an antibody or antigen-binding fragment thereof can specifically bind an epitope that a monoclonal antibody produced by a hybridoma cell line shown in Table 1 is directed to, using conventional reciprocal antibody competition assays. See e.g., Belanger et al. (1973), Clinica Chimica Acta 48:15.

Antibodies that competitively inhibit binding of one or more of SB1, SB2, SB3, SB4, SB5, SB6, SB7 or antigen-binding fragments thereof, reduce the binding of one or more of SB1, SB2, SB3, SB4, SB5, SB6, SB7 or antigen binding fragments thereof to a SAS1B polypeptide (e.g., a full-length SAS1B polypeptide or amino acids 24-163 of SEQ ID NO:26) or to cancer cells by about 40%, 50%, 75%, 90% or 100% in any type of competitive inhibition assay (see, e.g., Harlow and Lane, Antibodies, A Laboratory Manual, Cold Spring Harbor Publications, NY (1988)) are also antibodies of the present disclosure. Antibodies and antigen-binding fragments thereof can inhibit the binding SB1, SB2, SB3, SB4, SB5, SB6, or SB7 to human SAS1B.

Therefore, the present disclosure comprises antibodies that bind to an epitope that is substantially identical to or the same as an epitope of SAS1B to which an antibody produced by a hybridoma of Table 1 binds, and that can also comprise the activity of binding to cancer cells (e.g., uterine or pancreatic cancer cells), or binding to SAS1B or fragments thereof (e.g. amino acids 24-163 of SEQ ID NO:26).

### Amount of Binding

Antibodies of the present disclosure specifically bind SAS1B (e.g. human SAS1B). “Specifically binds” means that the antibody recognizes and binds to SAS1B with greater affinity than to other, non-specific molecules that are not SAS1B. For example, an antibody raised against an antigen (polypeptide) to which it binds more efficiently than to a non-specific antigen (e.g., a protein that is not related to or homologous to SAS1B) can be described as specifically binding to the antigen. Binding specificity can be tested using, for example, an enzyme-linked immunosorbant assay (ELISA), a radioimmunoassay (RIA), or a western blot assay using methodology well known in the art.

Antibodies of the present disclosure, antigen-binding fragments thereof, or variants thereof can specifically bind SAS1B with a wide range of disassociation constants (K_{d}). For example, an antibody can bind human SAS1B with a K_{d} equal to or less than about 10⁻⁷ M, such as but not limited to, 0.1-9.9×10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵ or any range or value therein, as determined by e.g., surface plasmon resonance or the Kinexa method. The present disclosure encompasses antibodies that bind human SAS1B polypeptides with a disassociation constant or K_{d} that is within any one of the ranges that are between each of the individual recited values. An antibody has the same or substantially identical activity as antibodies produced by the hybridomas shown in Table 1 when the K_{d} for binding to SAS1B (e.g., amino acids 24-163 of SEQ ID NO:26) is within about 0.1., 0.2, 0.3, 0.4, 0.5, 1, 2, 3, 4, 5, 6, 7, 8, 9, 10, 15, or 20% (or any range or particular value between 0.1 and 20%) of the K_{d} for binding to SAS1B (e.g., amino acids 24-163 of SEQ ID NO:26) of an antibody produced by the hybridomas shown in Table 1.

Antibodies of the present disclosure, antigen-binding fragments thereof or variants thereof can specifically bind human SAS1B polypeptides with an off rate (K_{off}) of less than or equal to 01.-9.9×10⁻³ sec⁻¹, 10⁻⁴ sec⁻¹, 10⁻⁵ sec⁻¹, 10⁻⁶ sec⁻¹, 10⁻⁷ sec⁻¹. The present disclosure encompasses antibodies that specifically bind SAS1B polypeptides with an off rate that is within any one of the ranges that are between each of the individual recited values. An antibody has the same or substantially identical activity as antibodies produced by the hybridomas shown in Table 1 when the K_{off} for binding to SAS1B (e.g., amino acids 24-163 of SEQ ID NO:26) is within about 0.1., 0.2, 0.3, 0.4, 0.5, 1, 2, 3, 4, 5, 6, 7, 8, 9, 10, 15, or 20% (or any range or particular value between 0.1 and 20%) of the K_{off} for binding to SAS1B (e.g., amino acids 24-163 of SEQ ID NO:26) of an antibody produced by the hybridomas shown in Table 1.

Antibodies of the present disclosure, antigen-binding fragments thereof, or variants thereof can specifically bind SAS1B polypeptides with an on rate (Kₒₙ) greater than or equal to 0.1-9.9×10³ M⁻¹ sec⁻¹, 10⁴ M⁻¹ sec⁻¹, 10⁵ M⁻¹ sec⁻¹, 10⁶ M⁻¹ sec⁻¹, 10⁷ M⁻¹ sec⁻¹, 10⁸ M⁻¹ sec⁻¹. The present disclosure encompasses antibodies that bind SAS1B polypeptides with on rate that is within any one of the ranges that are between each of the individual recited values. An antibody has the same or substantially identical activity as antibodies produced by the hybridomas shown in Table 1 when the Kₒₙ for binding to SAS1B (e.g., amino acids 24-163 of SEQ ID NO:26) is within about 0.1., 0.2, 0.3, 0.4, 0.5, 1, 2, 3, 4, 5, 6, 7, 8, 9, 10, 15, or 20% (or any range or particular value between 0.1 and 20%) of the Kₒₙ for binding to SAS1B (e.g., amino acids 24-163 of SEQ ID NO:26) of an antibody produced by the hybridomas shown in Table 1.

### Methods of Making Antibodies

Antibodies of the present disclosure can be produced using methods known to those of skill in the art. For example, an SAS1B antigen or a fragment thereof (e.g., amino acids 24-163 of SEQ ID NO:26) can be used to immunize animals. SAS1B or a fragment thereof can be conjugated to a carrier protein and/or administered to the animals with an adjuvant. An SAS1B antigen can comprise one or more epitopes (i.e., antigenic determinants). An epitope can be a linear epitope, sequential epitope or a conformational epitope. Epitopes within a polypeptide of the present disclosure can be identified by several methods. See, e.g., U.S. Pat. No. 4,554,101; Jameson & Wolf, CABIOS 4:181-186 (1988). For example, SAS1B can be isolated and screened. A series of short peptides, which together span the entire SAS1B polypeptide sequence, can be prepared by proteolytic cleavage. By starting with, for example, 100-mer polypeptide fragments, each fragment can be tested for the presence of epitopes recognized in an ELISA. For example, in an ELISA assay an SAS1B antigen, such as a 100-mer polypeptide fragment, is attached to a solid support, such as the wells of a plastic multi-well plate. A population of antibodies are labeled, added to the solid support and allowed to bind to the unlabeled antigen, under conditions where non-specific absorption is blocked, and any unbound antibody and other proteins are washed away. Antibody binding is detected by, for example, a reaction that converts a colorless substrate into a colored reaction product. Progressively smaller and overlapping fragments can then be tested from an identified 100-mer to map the epitope of interest.

Methods for preparing monoclonal antibodies from hybridomas are well known to those of skill in the art and include, e.g., standard cell culture methods and ascites production methods. Recombinant antibodies or fragments thereof produced by gene engineering can be made using the polynucleotide sequences of the present disclosure. Genes encoding antibodies or fragments thereof can be isolated from hybridomas of the present disclosure or other hybridomas. The genes can be inserted into an appropriate vector and introduced into a host cell. See, e.g., Borrebaeck & Larrick, Therapeutic Monoclonal Antibodies, Macmillan Publ. Ltd, 1990.

Antibodies can be produced using immunospot array assay on a chip (ISAAC) to obtain an antibody gene by screening single B cells, which secrete a specific monoclonal antibody, within several weeks (Jin et al., 2009 Nat. Med. 15, 1088-1092). Whole antibodies can also be made using PCR primers having VH or VL nucleotide sequences, a restriction site, and a flanking sequence to protect the restriction site to amplify the VH or VL sequences in scFv clones. Using well known cloning techniques, the PCR amplified VH domains can be cloned into vectors expressing a VH constant region, e.g., a human, rabbit or mouse constant region, and the PCR amplified VL domains can be cloned into vectors expressing a VL constant region, e.g., a human VL constant region or rabbit or murine light constant regions. The vectors for expressing the VH or VL domains can comprise, e.g., a promoter suitable to direct expression of the heavy and light chains in the chosen expression system, a secretion signal, a cloning site for the immunoglobulin variable domain, immunoglobulin constant domains, and a selection marker. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The nucleic acid sequences for human, rabbit, and mouse IgG constant regions have been cloned and sequenced and can be used to construct antibodies of the present disclosure.

Human antibodies can be made by sensitizing human lymphocytes with antigens of interest or cells expressing antigens of interest in vitro; and fusing the sensitized lymphocytes with human myeloma cells. Alternatively, a human antibody can be made by using an antigen to immunize a transgenic animal that comprises a partial or entire repertoire of human antibody genes. See Green et al., Nature Genetics 7:13-21 (1994); Mandez et al., Nature Genetics 15:146-156 (1997); Lonberg et al., Nature 368:856-859 (1994); WO 93/12227; WO 92/03918; WO 94/02602, WO 94/25585, WO 96/34096, and WO 96/33735).

Human antibodies can also be made by panning with a human antibody library. For example, the variable region of a human antibody is expressed as a single chain antibody (scFv) on the surface of a phage, using phage display method, and phages that bind to the antigen are selected. By analyzing the polynucleotides of selected phages, the polynucleotides encoding the variable regions of human antibodies that bind to the antigen can be determined. If the polynucleotide sequences of scFvs that bind to the antigen are identified, appropriate expression vectors comprising these sequences can be constructed, and then introduced into appropriate hosts and expressed to obtain human antibodies. See WO 92/01047, WO 92/20791, WO 93/06213, WO 93/11236, WO 93/19172, WO 95/01438, and WO 95/15388. These same human antibody production methods can be used to make mouse or rabbit antibodies.

Antibodies and fragments thereof can be purified by any method, including, e.g., protein A-Sepharose methods, hydroxyapatite chromatography, salting-out methods with sulfate, ion exchange chromatography, affinity chromatography, filtration, ultrafiltration, dialysis, preparative polyacrylamide gel electrophoresis, isoelectrofocusing or combinations thereof.

Antibodies can be dried or lyophilized (“freeze-dried) for more ready formulation into a desired vehicle/carrier where appropriate and for increased shelf-life.

### Conjugates

Antibodies of the present disclosure can be covalently attached to other molecules such that covalent attachment does not affect the ability of the antibody to bind to SAS1B or cells expressing SAS1B. For example, antibodies can be modified by, e.g., glycosylation, acetylation, pegylation, phosphorylation, amidation, derivatization by known protecting/blocking groups (e.g., methyl group, ethyl group, carbohydrate group), proteolytic cleavage, linkage to a cellular ligand or other protein.

Conjugated antibodies can be bound to various molecules including, for example, polymers, hyaluronic acid, fluorescent substances, luminescent substances, haptens, enzymes, metal chelates, cytotoxic agents, radionuclides, and drugs.

An anti-SAS1B antibody or antigen-binding fragment thereof can be modified with a moiety that improves its binding, stabilization and/or retention in circulation, e.g., in blood, serum, or other tissues, e.g., by at least 1.5, 2, 5, 10, or 50 fold. For example, the anti-SAS1B antibody or antigen-binding fragment thereof can be associated with (e.g., conjugated to) a polymer, e.g., a substantially non-antigenic polymer, such as a polyalkylene oxide, a polyethylene oxide, polyethylene glycol (PEG), polyethylenimine (PEI) modified with PEG (PEI-PEG), polyglutamic acid (PGA) (N-(2-Hydroxypropyl) methacrylamide (HPMA) copolymers. Suitable polymers will vary substantially by weight.

Polymers having molecular number average weights ranging from about 200 to about 35,000 Daltons (or about 1,000 to about 15,000, and 2,000 to about 12,500) can be used. For example, the anti-SAS1B antibody or antigen-binding fragment thereof can be conjugated to a water soluble polymer, a hydrophilic polyvinyl polymer, polyvinylalcohol or polyvinylpyrrolidone. Examples of such polymers include polyalkylene oxide homopolymers such as polyethylene glycol (PEG) (see e.g., Chapman et al., Nature Biotechnology, 17: 780 (1999), or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides. The antibodies or antigen-binding fragments thereof can also be conjugated to small molecules and other chemical moieties. Conjugated antibodies can be prepared by performing chemical modifications on the antibodies or fragments thereof. See e.g., U.S. Pat. Nos. 5,057,313 and 5,156,840.

### Variants of Antibodies

The constant region of an antibody or antigen-binding fragment thereof can be a human Fc region, e.g., a wild-type Fc region, or an Fc region that includes one or more amino acid substitutions. The constant region can have substitutions that modify the properties of the antibody (e.g., increase or decrease one or more of: Fc receptor binding, antibody glycosylation, the number of cysteine residues, effector cell function, or complement function). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies can also have mutations that stabilize the disulfide bond between the two heavy chains of an immunoglobulin, such as mutations in the hinge region of IgG4, as disclosed in the art (e.g., Angal et al. (1993) Mol. Immunol. 30: 105-08). See also, e.g., U.S. 2005/0037000.

The amino acid sequence of the heavy chain variable region (VH) or the light chain variable region (VL) in the antibody or antibody fragments can include modifications such as amino acid substitutions, deletions, additions, and/or insertions. For example, the modification may be in one or more of the CDRs of the anti-SAS1B antibody or antigen-binding fragment thereof. In certain embodiments, the modification involves one, two, or three amino acid substitutions in one or more CDRs and/or framework regions of the VH and/or VL domain of the anti-SAS1B antibody. Such substitutions are made to improve the binding, functional activity and/or reduce immunogenicity of the anti-SAS1B antibody. The amino acid substitutions can be conservative amino acid substitutions. In one embodiment, one, two, or three amino acids of the CDRs of the anti-SAS1B antibody or antigen-binding fragment thereof may be deleted or added as long as there is SAS1B binding and/or functional activity when VH and VL are associated.

The amino acid sequences of the CDRs are of primary importance for epitope recognition and antibody binding. Changes may be made to the amino acids that comprise the CDRs without interfering with the ability of the antibody to recognize and bind its cognate epitope. For example, changes that do not affect epitope recognition, yet increase the binding affinity of the antibody for the epitope may be made. Thus, also included in the scope of the present disclosure are improved versions of the disclosed antibodies, which also specifically recognize and bind SAS1B, preferably with increased affinity.

The effects of introducing one or more amino acid changes at various positions in the sequence of an antibody has been studied based on the knowledge of the primary antibody sequence, on its properties such as binding and level of expression. See, e.g., Yang et al., 1995, J. Mol. Biol., 254: 392; Rader et al., 1998, Proc. Natl. Acad. Sci. U.S.A., 95: 8910; Vaughan et al., 1998, Nature Biotechnology, 16:535.

For example, equivalents of a primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli. See Vaughan et al., 1998, Nature Biotechnology, 16: 535; Adey et al., 1996, Chapter 16, pp. 277-291, in “Phage Display of Peptides and Proteins”, Eds. Kay et al., Academic Press). These methods of altering the sequence of a primary antibody have resulted in improved affinities of the newly generated antibodies. Gram et al., 1992, Proc. Natl. Acad. Sci. U.S.A., 89: 3576; Boder et al., 2000, Proc. Natl. Acad. Sci. U.S.A., 97:10701; Davies & Riechmann, 1996, Immunotechnology, 2:169; Thompson et al., 1996, J. Mol. Biol., 256:77; Short et al., 2002, J. Biol. Chem., 277:16365; Furukawa, et al., 2001, J. Biol. Chem., 276:27622.

Using similar directed strategies of changing one or more amino acid residues of the antibody, the antibody sequences described herein can be used to develop anti-SAS1B antibodies with improved functions, including improved affinity for SAS1B.

The present disclosure also encompasses antibodies, fragments thereof, or variants thereof that have one or more of the same or substantially similar biological characteristics as the antibodies shown in Table 1. Biological characteristics are the in vitro or in vivo activities or properties of the antibodies shown in Table 1, including, for example, the ability to bind to SAS1B (e.g., amino acids 24-163 of SEQ ID NO:26) with a substantially similar K_{d}, K_{off}, and/or Kₒₙ rate, bind cancer cells, r cause death of cancer cells or combinations thereof.

Antibodies of the present disclosure can be used to generate anti-idiotype antibodies that “mimic” human SAS1B polypeptides using techniques well known to those skilled in the art. See, Greenspan & Bona, FASEB 17:437-444 (1993); Nissinoff, J. Immunol. 147:2429-2438 (1991).

One embodiment of the present disclosure provides mixtures of antibodies, antigen-binding fragments thereof, or variants thereof that bind to SAS1B, wherein the mixture has at least two, three, four, five or more different antibodies of the present disclosure.

The present disclosure also provides for panels of antibodies that have different affinities for SASB1, different specificities for SAS1B, or different dissociation rates. The present disclosure provides panels of at least about 2, 3, 4, 5, 6, 7, 10, 20, 50, 100, 250, 500, 750, or 1,000 antibodies.

In one embodiment of the present disclosure, the antibodies or antigen-binding fragments thereof are not naturally occurring due to one or more amino acid mutations in one or more constant regions or one or more framework regions or other mutations.

### Polynucleotides

Polynucleotides of the present disclosure contain less than an entire human, mouse or rabbit genome and can be single- or double-stranded nucleic acids. A polynucleotide can be RNA, DNA, cDNA, genomic DNA, chemically synthesized RNA or DNA or combinations thereof. The polynucleotides can be purified free of other components, such as proteins, lipids and other polynucleotides. For example, the polynucleotide can be 50%, 75%, 90%, 95%, 96%, 97%, 98%, 99%, or 100% pure by dry weight. Purity can be measured by a method such as column chromatography, polyacrylamide gel electrophoresis, or HPLC analysis. The polynucleotides of the present disclosure encode the polypeptides described above. In one embodiment of the present disclosure the polynucleotides encode polypeptides shown in, e.g., SEQ ID NOs:1-18 or portions or combinations thereof.

The polynucleotides of the present disclosure encode the polypeptides and antibodies of the present disclosure, as well as fragments thereof. A polynucleotide fragment can be about 9, 18, 21, 27, 30, 33, 39, 48, 51, 75, 100, 120, 130, 140, 150, 200 or more polynucleotides. One of skill in the art can obtain the polynucleotide sequences of the present disclosure using the polypeptide sequences and codon tables known to those of skill in the art. Polynucleotides can contain naturally occurring polynucleotides or sequences that differ from those of any naturally occurring sequences or polynucleotides (e.g., non-naturally occurring polynucleotides). Polynucleotides of the present disclosure can differ from naturally occurring nucleic acids, but still encode naturally occurring amino acids due to the degeneracy of the genetic code. Polynucleotides of the present disclosure can also comprise other heterologous nucleotide sequences, such as sequences coding for linkers, signal sequences, amino acid spacers, heterologous signal sequences, TMR stop transfer sequences, transmembrane domains, or ligands useful in protein purification such as glutathione-S-transferase, histidine tag, and staphylococcal protein A. Polynucleotides of the present disclosure can also comprise other nucleotide sequences.

Methods for introducing polynucleotides of the present disclosure (e.g., vectors comprising the polynucleotides or naked polynucleotides) into cells, either transiently or stably, are well known in the art. For example, transformation methods using standard CaCl₂), MgCl₂, or RbCl methods, protoplast fusion methods or transfection of naked or encapsulated nucleic acids using calcium phosphate precipitation, microinjection, viral infection, and electroporation.

In one embodiment of the present disclosure, a polynucleotide of the present disclosure is derived from a mammal, such as a human. Polynucleotides can also be synthesized in the laboratory, for example, using an automatic synthesizer. An amplification method such as PCR can be used to amplify polynucleotides from either genomic DNA or cDNA encoding the polypeptides. Polynucleotide molecules encoding a variant polypeptide can also be isolated by a gene amplification method such as PCR using a portion of a nucleic acid molecule DNA encoding a polypeptide shown in SEQ ID NOs:1-18 or fragments thereof as the probe.

Polynucleotides and fragments thereof of the present disclosure can be used, for example, as probes or primers to detect the presence of SAS1B polynucleotides in a sample, such as a biological sample. A biological sample can be, e.g., lymph node or tissue aspirate, serum, lymphocytes, whole blood, cellular suspension, plasma, circulating tumor cells, tumor cells or tissue, ascites fluid, urine, or fluid effusion. The ability of such probes to specifically hybridize to polynucleotide sequences will enable them to be of use in detecting the presence of complementary sequences in a given sample. Polynucleotide probes of the present disclosure can hybridize to complementary sequences in a sample such as a biological sample, for example, lymph tissue. Polynucleotides from the sample can be, for example, subjected to gel electrophoresis or other size separation techniques or can be dot blotted without size separation. The polynucleotide probes are preferably labeled. Suitable labels, and methods for labeling probes are known in the art, and include, for example, radioactive labels incorporated by nick translation or by kinase, biotin, fluorescent probes, and chemiluminescent probes. The polynucleotides from the sample are then treated with the probe under hybridization conditions of suitable stringencies.

The stringency of hybridization conditions for a polynucleotide encoding a variant polypeptide of the present disclosure to a polynucleotide encoding polypeptides shown in SEQ ID NOs:1-18 can be, for example, 10% formamide, 5×SSPE, 1× Denhart's solution, and 1× salmon sperm DNA (low stringency conditions). In one embodiment, the conditions are, 25% formamide, 5×SSPE, 1× Denhart's solution, and 1× salmon sperm DNA (moderate stringency conditions), and In another embodiment, the conditions are, 50% formamide, 5×SSPE, 1× Denhart's solution, and 1× salmon sperm DNA (high stringency conditions). However, several factors influence the stringency of hybridization other than the above-described formamide concentration, and one skilled in the art can suitably select these factors to accomplish a similar stringency. See e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989)). That is, a polynucleotide encoding a variant polypeptide of the present disclosure will hybridize to a polynucleotide encoding SEQ ID NOs:19-25 under low or high or both stringency conditions.

An isolated polynucleotide is a nucleic acid molecule that is not immediately contiguous with one or both of the 5′ and 3′ flanking sequences with which it is normally contiguous when present in a naturally occurring genome. Therefore, an isolated polynucleotide can be, for example, a polynucleotide that is incorporated into a vector, such as a plasmid or viral vector, a polynucleotide that is incorporated into the genome of a heterologous cell (or the genome of a homologous cell, but at a site different from that where it naturally occurs); and a polynucleotide that exists as a separate molecule such as a polynucleotide produced by PCR amplification, chemically synthesis, restriction enzyme digestion, or in vitro transcription. An isolated polynucleotide is also a nucleic acid molecule, such as a recombinant nucleic acid molecule that forms part of hybrid polynucleotide encoding additional polypeptide sequences that can be used for example, in the production of a fusion protein.

A polynucleotide can also comprise one or more expression control sequences such as promoters or enhancers, for example. A polynucleotide of the present disclosure can be present in a vector, such as, for example, an expression vector. If desired, polynucleotides can be cloned into an expression vector comprising, for example, promoters, enhancers, or other expression control sequences that drive expression of the polynucleotides of the present disclosure in host cells. The polynucleotides can be operably linked to the expression control sequences.

### Methods of Detection

One embodiment of the present disclosure provides methods of detecting SAS1B polypeptides in a sample. The methods comprise contacting the sample suspected of containing SAS1B polypeptides with an antibody or antigen binding portion thereof of the present disclosure to form SAS1B/antibody complexes. The presence of the SAS1B/antibody complexes are detected, thereby detecting the presence of the SAS1B polypeptides.

Another embodiment of the present disclosure provides a method of detection of SAS1B-positive cells (i.e., cells that express SAS1B on their surface) in a test sample comprising contacting one or more antibodies or antigen-binding portions thereof with the test sample under conditions that allow SAS1B-positive cell/antibody complexes to form. The cells can be permeabilized or cell lysates. The SAS1B positive cell/antibody complexes are then detected. The detection of SAS1B positive cell/antibody complexes is an indication that SAS1B cells are present in the test sample. The test sample can be, e.g., lymph node or tissue aspirate, serum, whole blood, cellular suspension, lymphocytes, plasma, circulating tumor cells, tumor cells or tissue, ascites fluid, urine, or fluid effusion. Polypeptide/antibody or SAS1B-positive cell/antibody complexes can be detected by any method known in the art, enzyme-linked immunosorbent assay (ELISA), multiplex fluorescent immunoassay (MFI or MFIA), radioimmunoassay (RIA), sandwich assay, western blotting, immunoblotting analysis, an immunohistochemistry method, immunofluorescence assay, fluorescence-activated cell sorting (FACS) or a combination thereof.

An immunoassay for SAS1B can utilize one antibody or several different antibodies. Immunoassay protocols can be based upon, for example, competition, direct reaction, or sandwich type assays using, for example, labeled antibody. Antibodies of the present disclosure can be labeled with any type of label known in the art, including, for example, fluorescent, chemiluminescent, radioactive, enzyme, colloidal metal, radioisotope and bioluminescent labels.

Antibodies of the present disclosure or antigen-binding portions thereof can be bound to a support and used to detect the presence of SAS1B. Supports include, for example, glass, polystyrene, polypropylene, polyethylene, dextran, nylon, amylases, natural and modified celluloses, polyacrylamides, agaroses and magletite.

Antibodies of the present disclosure can be used in a method of the diagnosis of a hyperproliferative disorder by obtaining a test sample from, e.g., a human or animal suspected of having a hyperproliferative disorder. The test sample is contacted with antibodies or antigen-binding portions thereof of the present disclosure under conditions enabling the formation of antibody-antigen complexes (i.e., immunocomplexes). One of skill in the art is aware of conditions that enable and are appropriate for formation of antigen/antibody complexes. The amount of antibody-antigen complexes (including, for example, a complex of an antibody or antigen-binding portion thereof and a cell expressing SAS1B on its surface) can be determined by methodology known in the art. A level that is higher than that formed in a control sample indicates the presence of a hyperproliferative disorder. A control sample is a sample that does not comprise any SAS1B polypeptides, SAS1B-positive cells, or antibodies specific for SAS1B. The amount of antibody/antigen complexes or antibodies bound to SAS1B-positive cells or cell lysates can be determined by methods known in the art.

A hyperproliferative disorder can be a neoplastic disorder (e.g., breast cancer, ovarian cancer, colorectal cancer, liver cancer, uterine cancer, pancreatic cancer, lung cancer, etc.) or a hematologic malignancy (e.g., leukemia, etc.).

In one embodiment of the present disclosure, a hyperproliferative disorder can be detected in a subject. A biological sample is obtained from the subject. One or more antibodies or antigen-binding portions thereof of the present disclosure are contacted with the biological sample under conditions that allow SAS1B polypeptide/antibody complexes (including, for example, a complex of an antibody or antigen-binding portion thereof and a cell expressing SAS1B on its surface) to form. The SAS1B polypeptide/antibody complexes are detected. The detection of the SAS1B polypeptide/antibody complexes is an indication that the mammal has a hyperproliferative disorder. The lack of detection of the polypeptide/antibody complexes is an indication that the mammal does not have a hyperproliferative disorder.

In one embodiment of the present disclosure, the SAS1B polypeptide/antibody complex is detected when an indicator reagent, such as an enzyme conjugate, which is bound to the antibody, catalyzes a detectable reaction. Optionally, an indicator reagent comprising a signal generating compound can be applied to the polypeptide/antibody complex under conditions that allow formation of a polypeptide/antibody/indicator complex. The polypeptide/antibody/indicator complex is detected. Optionally, the polypeptide or antibody can be labeled with an indicator reagent prior to the formation of a polypeptide/antibody complex. The method can optionally comprise a positive or negative control.

In one embodiment of the present disclosure, one or more antibodies of the present disclosure are attached to a solid phase or substrate. A test sample potentially comprising a polypeptide of the present disclosure is added to the substrate. One or more antibodies that specifically bind SAS1B are added. The antibodies can be the same antibodies used on the solid phase or can be from a different source or species and can be linked to an indicator reagent, such as an enzyme conjugate. Wash steps can be performed prior to each addition. A chromophore or enzyme substrate is added and color is allowed to develop. The color reaction is stopped and the color can be quantified using, for example, a spectrophotometer.

Assays of the present disclosure include, but are not limited to those based on competition, direct reaction or sandwich-type assays, including, but not limited to enzyme linked immunosorbent assay (ELISA), multiplex fluorescent immunoassay (MFI or MFIA) western blot, IFA, radioimmunoassay (RIA), western blot, hemagglutination (HA), fluorescence polarization immunoassay (FPIA), fluorescence-activated cell sorting (FACS), and microtiter plate assays (any assay done in one or more wells of a microtiter plate).

Assays can use solid phases or substrates or can be performed by immunoprecipitation or any other methods that do not utilize solid phases. Where a solid phase or substrate is used, one or more antibodies or antigen-binding portions thereof of the present disclosure are directly or indirectly attached to a solid support or a substrate such as a microtiter well, magnetic bead, non-magnetic bead, column, matrix, membrane, fibrous mat composed of synthetic or natural fibers (e.g., glass or cellulose-based materials or thermoplastic polymers, such as, polyethylene, polypropylene, or polyester), sintered structure composed of particulate materials (e.g., glass or various thermoplastic polymers), or cast membrane film composed of nitrocellulose, nylon, polysulfone or the like (generally synthetic in nature). All of these substrate materials can be used in suitable shapes, such as films, sheets, or plates, or they may be coated onto or bonded or laminated to appropriate inert carriers, such as paper, glass, plastic films, or fabrics. Suitable methods for immobilizing peptides on solid phases include ionic, hydrophobic, covalent interactions and the like.

In one type of assay format, one or more antibodies or antigen-binding portions thereof can be coated on a solid phase or substrate. A test sample suspected of containing SAS1B polypeptides or SAS1B-positive cells is incubated with an indicator reagent comprising a signal generating compound conjugated to an antibody or antigen-binding antibody fragment specific for SAS1B (indicator reagent composition) for a time and under conditions sufficient to form antigen/antibody complexes of either SAS1B polypeptides of the test sample to the antibodies or antigen-binding fragments thereof of the solid phase or the indicator reagent compound. The reduction in binding of the indicator reagent can be quantitatively measured. A measurable reduction in the signal compared to the signal generated from a confirmed negative SAS1B test sample indicates the presence of SAS1B in the test sample. This type of assay can quantitate the amount of SAS1B in a test sample.

The formation of a polypeptide/antibody complex or a polypeptide/antibody/indicator complex (including, for example, a complex of an antibody or antigen-binding portion thereof and a cell expressing SAS1B on its surface or a complex of an antibody or antigen-binding portion thereof, an indicator reagent, and a cell expressing SAS1B on its surface) can be detected by e.g., radiometric, colorimetric, fluorometric, size-separation, or precipitation methods. Optionally, detection of a polypeptide/antibody complex is by the addition of a secondary antibody that is coupled to an indicator reagent comprising a signal generating compound. Indicator reagents comprising signal generating compounds (labels) associated with a polypeptide/antibody complex can be detected using the methods described above and include chromogenic agents, catalysts such as enzyme conjugates fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like. Examples of enzyme conjugates include alkaline phosphatase, horseradish peroxidase, beta-galactosidase, and the like. The selection of a particular label is not critical, but it will be capable of producing a signal either by itself or in conjunction with one or more additional substances.

Formation of the complex can be indicative of the presence of SAS1B-positive cells in a test sample. Therefore, the methods of the present disclosure can be used to diagnose a hyperproliferative disease in a mammal.

The methods of the present disclosure can also indicate the amount or quantity of SAS1B in a test sample. With many indicator reagents, such as enzyme conjugates, the amount of SAS1B present is proportional to the signal generated. Depending upon the type of test sample, it can be diluted with a suitable buffer reagent, concentrated, or contacted with a solid phase without any manipulation. For example, it usually is preferred to test samples that previously have been diluted, or concentrated specimens, in order to determine the presence and/or amount of SAS1B present.

### Vectors and Host Cells

A polypeptide can be expressed in systems, e.g., cultured cells, which result in substantially the same post-translational modifications present as when the polypeptide is expressed in a native cell, or in systems that result in the alteration or omission of post-translational modifications, e.g., glycosylation or cleavage, present when expressed in a native cell.

An expression vector can be, for example, a plasmid, such as pBR322, pUC, or ColE1, or an adenovirus vector, such as an adenovirus Type 2 vector or Type 5 vector. Vectors suitable for use in the present disclosure include, for example, bacterial vectors, mammalian vectors, viral vectors (such as retroviral, adenoviral, adeno-associated viral, herpes virus, simian virus 40 (SV40)) and baculovirus-derived vectors for use in insect cells. Polynucleotides in such vectors are preferably operably linked to a promoter, which is selected based on, e.g., the cell type in which expression is sought.

The expression vector can be transferred to a host cell by conventional techniques and the transfected cells are then cultured by conventional techniques to produce an antibody of the present disclosure. The present disclosure includes host cells containing polynucleotides encoding an antibody of the present disclosure (e.g., whole antibody, a heavy or light chain thereof, or portion thereof, or a single chain antibody of the present disclosure, or a fragment or variant thereof), operably linked to a heterologous promoter. For the expression of entire antibody molecules, vectors encoding both the heavy and light chains may be co-expressed in the host cell for expression of the entire immunoglobulin molecule.

Host cells into which vectors, such as expression vectors, comprising polynucleotides of the present disclosure can be introduced include, for example, prokaryotic cells (e.g., bacterial cells) and eukaryotic cells (e.g., yeast cells; fungal cells; plant cells; insect cells; and mammalian cells). Such host cells are available from a number of different sources that are known to those skilled in the art, e.g., the American Type Culture Collection (ATCC), Manassas, Va. Host cells into which the polynucleotides of the present disclosure have been introduced, as well as their progeny, even if not identical to the parental cells, due to mutations, are included in the present disclosure. Host cells can be transformed with the expression vectors to express the antibodies or antigen-binding fragments thereof. Host cells expressing antibodies or antigen-binding fragments thereof of the present disclosure include cells and hybridomas transformed with a polynucleotide of the present disclosure.

One embodiment of the present disclosure provides methods of producing a recombinant cell that expresses an SAS1B antibody, antigen-binding fragment thereof or portion thereof, comprising transfecting a cell with a vector comprising a polynucleotide of the present disclosure. An SAS1B antibody, or fragment, or portion thereof, can then be produced by expressing the polypeptide in the recombinant host cell.

Isolation and purification of polypeptides produced in the systems described above can be carried out using conventional methods, appropriate for the particular system. For example, preparative chromatography and immunological separations employing antibodies, such as monoclonal or polyclonal antibodies, can be used.

### Antibody-Drug Conjugates

Antibodies and antigen-binding fragments thereof of the present disclosure that specifically bind SAS1B can be conjugated to a therapeutic agent or effector molecule to form an “antibody-drug conjugate”. A therapeutic agent is an agent with a biological activity directed against a particular target molecule or a cell bearing a target molecule. Therapeutic agents can include, for example, various drugs such as vinblastine, daunomycin, cytotoxins such as maytansinoids and maytansinoid analogs, a prodrug, tomaymycin derivatives, taxoids, a leptomycin derivative, CC-1065 and CC-1065 analogs, encapsulating agents (such as liposomes) that contain pharmacological compositions, therapeutic agents, toxins (e.g., ricin, abrin, diphtheria toxin and subunits thereof, botulinum toxins A through F, variants of toxins (see, e.g., U.S. Pat. Nos. 5,079,163 and 4,689,401), Pseudomonas exotoxin (PE) (see e.g., U.S. Pat. No. 5,602,095) and variants thereof (see, e.g. U.S. Pat. Nos. 4,892,827; 5,512,658; 5,602,095; 5,608,039; 5,821,238; and 5,854,044; PCT Publication No. WO 99/51643; Pai et al., Proc. Natl. Acad. Sci. USA 88:3358, 1991; Kondo et al., J. Biol. Chem. 263:9470, 1988; Pastan et al., Biochim. Biophys. Acta 1333:C1-C6, 1997)), radioactive agents such as ¹²⁵I, ³²P, ¹⁴C, ³H and ³⁵S and other labels, target moieties and ligands. An effector molecule is a small molecule that selectively binds to a protein and regulates its biological activity.

Effector or therapeutic molecules can be linked to an antibody of the present disclosure using any number of means known to those of skill in the art, for example by covalent or noncovalent attachment. Therapeutic agents or effector molecules that are polypeptides will typically contain a variety of functional groups; such as carboxylic acid (COOH), free amine (—NH₂) or sulfhydryl (—SH) groups, which are available for reaction with a suitable functional group on an antibody to result in the binding of the effector molecule or therapeutic agent. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule or therapeutic agent. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule or therapeutic agent from the antibody when the antibody-drug conjugate has reached its target site. Therefore, antibody-drug conjugates can comprise linkages that are cleavable in the vicinity of the target site. Cleavage of the linker to release the effector molecule or therapeutic molecule from the antibody can be accomplished by, for example, enzymatic activity or conditions to which the antibody-drug conjugate is subjected either inside the target site or in the vicinity of the target site.

In view of the large number of methods that have been reported for attaching a variety of radiodiagnostic compounds, radiotherapeutic compounds, labels (such as enzymes or fluorescent molecules) drugs, toxins, and other agents to antibodies one skilled in the art will be able to determine a suitable method for attaching a given therapeutic agent or effector molecule to an antibody or other polypeptide.

Antibodies of the present disclosure can be labeled with a detectable moiety. Detectable moieties include, for example, fluorescent compounds, including fluorescein, fluorescein isothiocyanate, rhodamine, 5-dimethylamine-1-napthalenesufonyl chloride, phycoerythrin, lanthanide phosphors, bioluminescent markers (e.g., luciferase, green fluorescent protein (GFP), yellow fluorescent protein (YFP)), enzymes (e.g., horseradish peroxidase, β-galactosidase, luciferase, alkaline phosphatase, glucose oxidase), a magnetic agent (e.g. gadolinium), lanthanides (e.g., europium and dysprosium), manganese, paramagnetic particles (e.g., superparamagnetic iron oxide), polypeptide epitopes recognized by a secondary reporter (such as leucine zipper pair sequences, radiolabeled amino acids, binding sites for secondary antibodies, metal binding domains, epitope tags). Detectable moieties can be attached to antibodies by spacer arms of various lengths to reduce potential steric hindrance.

### Methods of Treatment

Antibodies and antigen-binding fragments thereof can be conjugated to a cytotoxic agent, such as duocarmycin, maytansanoids, and auristatins, to form a drug having specific cytotoxicity towards SAS1B-expressing cells by targeting the drug to SAS1B. Cytotoxic conjugates comprising such antibodies and/or antigen-binding fragments thereof and a drug or cytotoxin can be used as a therapeutic for treatment of hyperproliferative disorders such as any SAS1B-positive cancer, including but not limited to, uterine cancer, pancreatic cancer, ovarian cancer.

One embodiment of the present disclosure provides methods of treating or preventing hyperproliferative disorders comprising administering an effective amount of an antibody or antigen-binding fragment thereof of the present disclosure or an antibody-drug conjugate of the present disclosure to a mammal in need thereof.

An “effective amount” is an amount sufficient to effect beneficial or desired results. For example, an effective amount is one that achieves the desired therapeutic effect. An effective amount can be administered in one or more administrations, applications or dosages. An effective amount of a pharmaceutical composition (i.e., an effective dosage) depends on the pharmaceutical composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with an effective amount of the pharmaceutical compositions described herein can include a single treatment or a series of treatments.

“Treatment” means the administration of one or more pharmaceutical compositions to a subject. The term treatment also includes an adjustment (e.g., increase or decrease) in the dose or frequency of one or more pharmaceutical agents that a subject can be taking, the administration of one or more new pharmaceutical agents to the subject, or the removal of one or more pharmaceutical agents from the subject's treatment plan. Treatment also refers to any amelioration of one or more symptoms of a hyperproliferative disease, improvement in patient survival, or the reversal of the disease.

A subject can be an animal, for example, a mammal, a human, monkey, dog, cat, horse, cow, pig, goat, rabbit, or mouse.

A “pharmaceutical composition” is a sterile or aseptic composition of an antibody or antigen-binding fragment thereof or antibody-drug conjugate of the present disclosure formulated with a pharmaceutically acceptable carrier, which can be safely administered to a subject. The pharmaceutical composition does not cause undesirable side effects when administered to a patient that outweigh the beneficial effects.

One embodiment of the present disclosure provides a pharmaceutical composition for the treatment of a hyperproliferative disorder in a mammal, which comprises an effective amount of an antibody or antigen-binding portion thereof or an antibody-drug conjugate of the present disclosure and a pharmaceutically acceptable carrier. The pharmaceutical composition can be used for the treatment of cancer, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma, and other cancers yet to be determined in which SAS1B is expressed. The instant present disclosure provides pharmaceutical compositions comprising: an effective amount of an antibody, antibody fragment or antibody-drug conjugate of the present disclosure, and a pharmaceutically acceptable carrier, which may be inert or physiologically active.

“Pharmaceutically-acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. Isotonic agents, such as sugars, polyalcohols, or sodium chloride can be present in compositions of the present disclosure. Examples of suitable carriers include, for example: Dulbecco's phosphate buffered saline, pH about 7.4, containing or not containing about 1 mg/ml to 25 mg/ml human serum albumin; 0.9% saline (0.9% w/v sodium chloride (NaCl)), and 5% (w/v) dextrose. Carriers can also contain an antioxidant such as tryptamine and a stabilizing agent such as TWEEN20® (polysorbate).

Administration can be by any method, including, for example parenteral (e.g. intravenous, intramuscular, intraperinoneal, subcutaneous, intra-articular, intrasynovial, intratumoral, peritumoral, intralesional, or perilesional). Compositions of the present disclosure can be administered intravenously as a bolus or by continuous infusion over a period of time.

Sterile compositions for parenteral administration can be prepared by incorporating the antibody, antigen-binding fragment or antibody-drug conjugate of the present disclosure in the required amount in the appropriate solvent, followed by sterilization by microfiltration. As solvent or vehicle include, for example, water, saline, phosphate buffered saline, dextrose, glycerol, ethanol, and the like, as well as a combination thereof. Isotonic agents, such as sugars, polyalcohols, or sodium chloride can be present in compositions of the present disclosure. These compositions may also contain adjuvants, in particular wetting, isotonizing, emulsifying, dispersing and stabilizing agents. Sterile compositions for parenteral administration can also be prepared in the form of sterile solid compositions, which can be dissolved at the time of use in sterile water or any other injectable sterile medium.

The antibodies, antigen-binding fragments thereof or antibody-drug conjugates of the present disclosure can also be orally administered as a solid composition (tablets, pills, powders gelatin capsules, sachets) or granules) or liquid compositions (pharmaceutically acceptable solutions, suspensions, emulsions, syrups and elixirs containing inert diluents such as water, ethanol, glycerol, vegetable oils or paraffin oil). These compositions may comprise substances other than diluents, for example wetting, sweetening, thickening, flavoring or stabilizing products.

The doses depend on the desired effect, the duration of the treatment and the route of administration used; they are generally between 5 mg and 1000 mg per day orally for an adult with unit doses ranging from 1 mg to 250 mg of active substance.

Antibodies, antibody-binding portions thereof, or antibody-drug conjugates of the present disclosure can be used for the treatment of a hyperproliferative disorder in a mammal. The antibodies, antibody-binding portions thereof, or antibody-drug conjugates of the present disclosure can also be used to treat the neovascularization of a tumor.

Similarly, the present disclosure provides a method for inhibiting the growth of selected cell populations comprising contacting target cells, or tissue containing target cells, with an effective amount of an antibody, antigen-binding fragment or antibody-drug conjugate of the present disclosure, or an antibody, antigen-binding fragment or a therapeutic agent comprising a cytotoxic conjugate, either alone or in combination with other cytotoxic or therapeutic agents.

Methods for inhibiting the growth of selected cell populations expressing SAS1B can be practiced in vitro, in vivo, or ex vivo. “Inhibiting growth” means slowing the growth of a cell, decreasing cell viability, causing the death of a cell, lysing a cell and inducing cell death, over a short period of time (e.g., minutes to hours) or a long period of time (e.g., days to months).

Examples of in vitro uses include treatments of autologous bone marrow prior to their transplant into the same patient in order to kill diseased or malignant cells; treatments of bone marrow prior to its transplantation in order to kill competent T cells and prevent graft-versus-host-disease (GVHD); treatments of cell cultures in order to kill all cells except for desired variants that do not express the target antigen; or to kill variants that express undesired antigen.

Examples of clinical ex vivo use include the removal of tumor cells or lymphoid cells from bone marrow prior to autologous transplantation in cancer treatment or in treatment of autoimmune disease, or to remove T cells and other lymphoid cells from autologous or allogeneic bone marrow or tissue prior to transplant in order to prevent graft versus host disease (GVHD).

For clinical in vivo use, the antibody, the antigen-binding fragment, or the antibody-drug conjugate of the present disclosure can be supplied as solutions that are sterile and contain appropriate levels of endotoxin. Examples of suitable protocols of antibody-drug conjugate administration are as follows. Antibodies, antigen-binding fragments thereof or antibody-drug conjugates can be given weekly for 4 weeks as an i.v. bolus each week. Bolus doses are given in 50 to 100 ml of normal saline to which 5 to 10 ml of human serum albumin can be added. Dosages can be 10 μg to 100 mg per administration, i.v. (range of 100 ng to 1 mg/kg per day). Dosages can range from 50 μg to 30 mg. Dosages can range from 1 mg to 20 mg. After four weeks of treatment, the patient can continue to receive treatment on a weekly basis. Specific clinical protocols with regard to route of administration, excipients, diluents, dosages, times, etc., can be determined by one of ordinary skill in the art as the clinical situation warrants.

The antibodies or antigen-binding fragments of the present disclosure can also be used to detect SAS1B in a biological sample in vitro or in vivo. Antibodies or fragments thereof of the present disclosure can be used to determine the level of SAS1B in a tissue or in cells derived from the tissue. The tissue can be diseased tissue, a tumor or a biopsy of a tumor. The tissue or biopsy thereof can be frozen, fixed, permeabilized or non-permeabilized.

The above-described method can be used to diagnose a cancer in a subject known to or suspected to have a cancer, wherein the level of SAS1B measured in said patient's tissues, blood or serum is compared with that of a normal reference subject or standard. The method can then be used to determine whether a tumor or cells of tissue of the patient expresses SAS1B, which may suggest that the tumor or patient will respond well to treatment with the antibodies, antigen-binding fragments or antibody-drug conjugates of the present disclosure.

A method for diagnosis is also provided in which labeled antibodies, antigen-binding fragments thereof, or antibody-drug conjugates are administered to a subject suspected of having a cancer, and the distribution of the label within the body of the subject is measured or monitored.

All patents, patent applications, and other scientific or technical writings referred to anywhere herein are incorporated by reference herein in their entirety. The present disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by embodiments, optional features, modification and variation of the concepts herein disclosed may be resorted to by those skilled in the art, and that such modifications and variations are considered to be within the scope of the present disclosure as defined by the description and the appended claims.

In addition, where features or aspects of the present disclosure are described in terms of Markush groups or other grouping of alternatives, those skilled in the art will recognize that the present disclosure is also thereby described in terms of any individual member or subgroup of members of the Markush group or other group.

The following are provided for exemplification purposes only and are not intended to limit the scope of the present disclosure described in broad terms above.

### EXAMPLES

### Example 1 Production and Characterization of SAS1B and Antibodies

The human SAS1B protein is encoded by the AstI gene (astacin-like metalloendopeptidase). ASTL expression is upregulated in lung adenocarcinomas. ASTL has a gain of copy number ranking of 1389, placing ASTL in the top 8% of genes upregulated in lung adenocarcinomas. This data in primary tumors is supported by upregulation of ASTL in lung adenosquamous and adenocarcinoma cell lines. ASTL expression was confirmed in several squamous and adenocarcinoma lung cancer cell lines. PCR amplification of the human SAS1B catalytic domain of 579 bp occurred in lung cancer cell lines NCI-H226, A549, HEK-293, human ovary, MMMT539, while the control water was negative. Western blotting of protein extracts from lung cancer H226 and A549 cell lines using a guinea pig polyclonal anti-human SAS1B antibody showed expression of full length (46 kD) and truncated SAS1B proteins in both lung cancer cell lines H226 and A549. Most importantly, SAS1B can be localized to the surfaces of both H226 and A549 cells. Immunofluorescent staining of unpermeabilized A549 cells and cell surface staining of H226 cells was observed. Polyclonal antibodies to SAS1B in the presence of complement lyse (kill) tumor cells that express SAS1B, while inactivated complement has no effect.

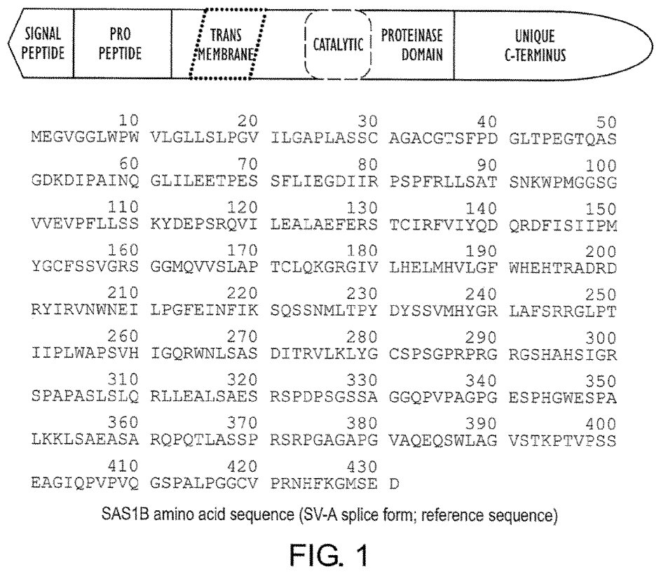

A diagram of the human SAS1B polypeptide is shown in FIG. 1. SAS1B has been divided into domains. These include a classic signal peptide, propeptide, putative transmembrane, catalytic region embedded in overall larger protease domain and C-terminus domain. SAS1B shows protein microheterogeneity in both mice and humans.

SAS1B has several splice variant forms: SV-A, SV-B, SV-C, SV-D, SV-E, and SV-F. Splice variants A and C were PCR amplified using variant specific forward and reverse primers. SV-A and SV-C were amplified from the following RNA sources: 1) SNU539 cell line (malignant mixed Mullerian tumor); 2) pancreatic ductal adenocarcinoma (PDAC) mouse xenograft tumor samples (human primary patient tumors were affixed into pancreas of nude mice and then propagated; tumor RNA interrogated here from approximately F5 generation); 3) human primary patient head and neck squamous cell carcinoma (HNSCC) samples. PCR products were subcloned and sequenced and sequence identity was used to confirm SV-A, SV-C, or SV-D splice variants.

Splice variant B was identified by sequencing data generated by subcloning PCR products from SV-A primer set. DNA sequences were identified from all three RNA sources listed above confirming presence of SV-B transcript in different tumor types.

Splice variant D was PCR amplified using variant specific primers from SNU539 cell line RNA. Identification was confirmed by subcloning and sequencing PCR amplicon.

These results show identification of splice variants A, B, C, and D transcripts present in various cancer types.

Amplification of each of the splice variants reveals that SV-B lacks 18 amino acids from Exon 8, SV-C has a novel N-terminal transmembrane domain, SV-D has a novel transmembrane domain and an ORF stop after Exon 7. SV-E and SV-F have a novel transmembrane domain and an N-terminal ORF stop after Exon 4. SV-E and SV-F have a C-terminal ORF that starts from extended Exon 5.

Amino acids 22 to 431 of splice variant A of SAS1B was expressed in in E. coli, and used to generate mouse antibodies SB1, SB2, SB3, SB4, SB5, SB6, and SB7. The immunogen was denatured prior to generation of antibodies.

A western blot of human SAS1B and mouse SAS1B was probed with anti-human SAS1B murine monoclonal antibodies SB1, SB2, SB3, SB4, SB5, SB6, and SB7. SAS1B monoclonal antibodies SB2, SB3, SB4, SB5, SB6, and SB7 are specific to the human SAS1B only. The SB1 monoclonal antibody reacts with both human and mouse SAS1B. The SAS1B monoclonal antibodies (SB1, SB2, SB3, SB4, SB5, SB6, and SB7) demonstrated specificity in a western blot assay for human SAS1B antigen in an E. coli protein background.

Uterine cancer cell line (SNU539) was extracted in RIPA buffer containing protease inhibitor and 42 ug of total protein was loaded per lane, resolved by SDS-PAGE, transferred to a membrane, and probed with anti-human SAS1B monoclonal antibodies (“mAbs”) at 3 ug/ml. The membrane was washed, probed with goat-Anti-mouse (Fab)2-HRP at 0.2 ug/ml and developed with TMB. SB4 and SB5 mAbs identified a predominant band of ˜43 kD. The SB2 mAb identified a ˜43 kD band, a ˜52 kD band, and a minor band of ˜50 kD. The additional band reactivity by SB2 mAb suggests that complementary binding region (CDR) of SB2 is different from that of SB4 and SB5. The data also suggests that SAS1B expression in SNU539 is heterogeneous.

2-D gel electrophoresis and western blotting using 17 cm IPG (immobilized pH gradient) strips were performed. SNU539 cell lysate in Celis (8 M urea, 2 M thiourea, 2% NP-40 and 100 mM DTT and 0.2% ampholines) was prepared. 400 mg of the protein per IPG strip was loaded as follows: −pH 4-7 NL, 17 cm. The proteins resolved by IEF (isoelectric focusing) were further separated by SDS-PAGE using 12% gels. After transblotting the proteins, the membranes were probed with polyclonal and monoclonal antibodies with appropriate controls. An SAS1B rabbit polyclonal antibody recognized mainly a ˜65 kDa band, a ˜50 kDa band (pl 4.5), and a ˜37 kDa (pl 7) band on a 2D western blot in which the lysate was prepared in Celis. The mouse monoclonal SB5 mainly recognized several charge variants at around 32 kDa.

SAS1B monoclonal antibodies were analyzed with mammalian cell-expressed recombinant SAS1B by western blotting. All the monoclonal antibodies (SB1, SB2, SB3, SB4, SB5, SB6, and SB7) reacted with the full-length SAS1B splice variant A polypeptide and none of the them reacted with the SAS1B extracellular domain (aa 164-431) polypeptide suggesting that their epitope lies in the N-terminus of the protein. However, the rabbit polyclonal antibody reacted with both full-length as well as the truncated SAS1B.

Experiments were conducted to determine the specificity of anti-human SAS1B monoclonal antibodies SB1, SB2, SB3, SB4, SB5, SB6 and SB7 against the human SAS1B protein with a C-terminal V5 tag expressed by HEK293T cells by double immuno-fluorescence from two different SAS1B epitopes. HEK293T cells were seeded at ˜100,000 per well of a 24 well plate containing a lysine-coated 12 mm coverslip. 24 hours later, the cells were transfected with 1 ug of SV-A plasmid with 2 ul of TURBOFECT® transfection reagent followed by no change of media. 48 h later the cells were PFA (paraformaldehyde) fixed and methanol permeabilized for an IF study as follows.

The cells were removed from cell culture media and fixed in 4% PFA in PBS, 300 ul for 15 min. The cells were washed in 1 ml PBS in wells for 3 times and permeabilized in methanol (100%) for 15 min in 0.5 ml at RT. The cells were washed in PBS 1 ml for 3 times and blocked in complete culture media (DMEM) with 5% normal goat serum (NGS) for 30 min (i.e. BB, blocking buffer). The first antibody in BB was added for 1 h at 10 ug/ml for mouse monoclonal antibodies (SB1, SB2, SB3, SB4, SB5, SB6, SB7) and 5 ug/ml for anti-V5 DYLIGHT® 488 (green) rabbit (a rabbit polyclonal antibody to V5 tag). The cells were washed in PBS 3 times. The second antibody in BB (Goat anti-mouse Alexa Flour 594, red) was added for 1 h at 5 ug/ml (1:200 dilution). The cells were washed in PBS 3 times. The cells were mounted in slow fade DAPI 3 ul and stored at 4° C. before confocal imaging.

The mock transfected HEK293T cells showed no V5 signal or SAS1B signal when probed with anti-V5 or anti-SAS1B mAbs, SB1, SB2, SB3, SB4, SB5, SB6, SB7 or mouse IgG control antibody or the secondary antibody alone. The anti-V5 antibody confirmed expression of human SAS1B with C-terminal V5 tag in fixed and permeabilized HEK293T cells. However, lack of a signal for SB1 suggested loss of SB1 specific epitope, possibly due to fixation with 4% paraformaldehyde (PFA).

The V5 tag antibody confirmed the expression of SAS1B SV-A epitopes in transfected HEK293T cells. Anti-human SAS1B mAbs SB2, SB3, SB4, SB5, SB6, SB7 IF colocalize (merge) with V5-tag epitope signal confirming the specificity of SB2, SB3, SB4, SB5, SB6, and SB7 mAb for the SAS1B epitope. Therefore, double epitope probing of HEK293T cells transfected with human SAS1B SV-A containing V5 epitope revealed colocalization of IF pattern from SB2, SB3, SB4, SB5, SB6, and SB7 mouse mAbs with V5 tag antibody. The IF pattern confirmed the specificity of these mouse mAbs towards human SAS1B protein in fixed and permeabilized cells.

### Example 2 Live Immunofluorescence Assays

Anti-Human SAS1B mouse monoclonal antibodies reactive to SAS1B epitope on live cancer cell surfaces were identified by indirect immuno-fluorescence (IF) for the development of therapeutic antibodies. M1 (renal cancer cell line) and MMMT539 (uterine cancer cell line) cells were seeded at 20,000 cells in 1 ml per well on collagen coated coverslips in 24 well plates. The media was replaced 22 h post seeding. For antibody probing, cells were kept on ice for ˜8 to 18 min following 47 h post-seeding. The first antibody was added at 10 ug/ml, 20 ug/ml or 50 ug/ml in complete culture media with 15 mM NaN3 (pre-chilled), incubated on ice for 60 min, and the cells washed in complete culture media with 15 mM NaN3 in cold 1 ml, ×3. The second antibody, goat-antimouse-AF488 (pre-chilled), was added to the wells at 5 ug/ml in complete culture media with 15 mM NaN3 for 60 min on ice. The cells were washed in complete media with 15 mM NaN3, 1 ml, ×3 on ice. The cells were fixed in 4% PFA in PBS for 20 minutes on ice. The cells were washed in distilled water at RT, 1 ml, ×1 and mounted in 8 ul of SLOWFADE® Diamond Antifade Mountant with DAPI. An anti-EGFR monoclonal antibody was used as a positive control on live M1 cells. Epidermal growth factor receptor (EGFR) is a transmembrane glycoprotein and member of protein kinase superfamily. Both fluorescence and confocal microscopy confirmed the EGFR expression on the cell surface of live M1 renal carcinoma cell line. Most of the cells expressed the receptor and validated the live cell surface IF staining methodology for the screening of anti-human SAS1B mouse monoclonal antibodies. The receptor molecule was found on the cell surfaces. Anti-human CABYR monoclonal antibody 3A4 was used as a negative control on live MMMT539 cells. CABYR is a calcium binding cytoskeleton sperm protein with no transmembrane domain. Anti-CABYR mAb, 3A4 showed no fluorescence signal on live MMMT539 cancer cell line by fluorescence or confocal microscopy and was used as a negative control.

SB1, SB2, SB3, SB4, and SB5 showed clear fluorescence signal on the surface of live uterine tumor cells like the anti-EGFR mAb, suggesting that the SAS1B N-terminal epitope, upstream of residue 164 being exposed to the cell surface. The signal was observed all over the cell surface including the delicate processes at the cell periphery indicating the exposure of the SAS1B N-terminus.

Anti-human SAS1B mAbs, SB1, SB2, SB3, SB4 and SB5 labelled the surface these SAS1B-expressing cancer cells suggesting that the SAS1B N-terminal epitope being exposed to these cancer cell surfaces. Surface labelling of these two cancer cell lines by these mAbs suggests that SB1, SB2, SB3, SB4 and SB5 could be used as a targeting agent to kill these cancer cells.

Live MPanc96 cells (pancreatic ductal adenocarcinoma cell line) were incubated with different anti-SAS1B monoclonal antibodies (as well as DAPI stain) for nucleus localization. Cells were fixed and permeabilized after primary and secondary antibody incubation thus fluorescence staining observed represents surface localized SAS1B. Monoclonal antibodies SB4 and SB5 were used, as well as normal rabbit or mouse IgG's as a negative control. All primary antibodies were used at a concentration of 20 ug/mL. SAS1B mAbs SB4 and SB5 were shown to immunoreact with cell surface localized SAS1B in pancreatic cancer cells. Similar results were obtained for an additional PDAC cell line known as 366. mAbs SB4 and SB5 therefore recognize cell surface SAS1B in pancreatic cancer cell lines.

### Example 3 Cytotoxicity Assays

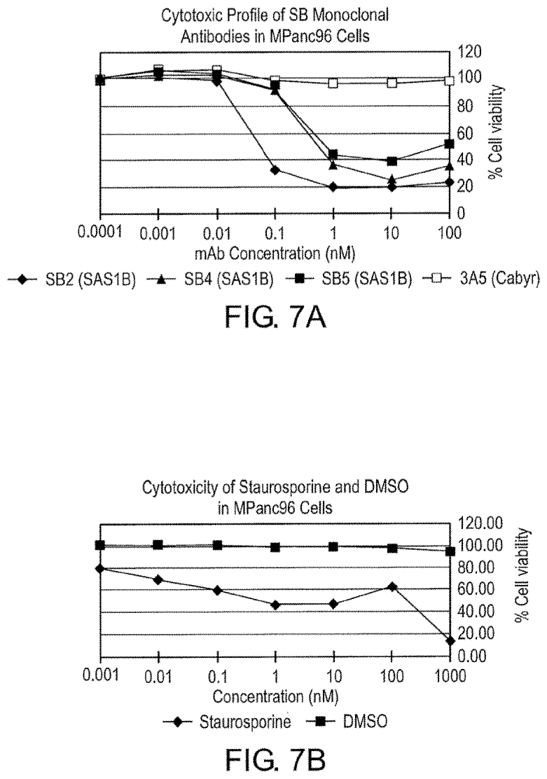

The cytotoxic effect of anti-human SAS1B mAbs SB2, SB4, SB5 on human pancreatic cancer cell MPanc96 was determined by ADC assay. Human pancreatic cancer cells MPanc96 were seeded in triplicate on 96 well plates (1100 cells per well) overnight and then treated with SB2, SB4, SB5, and 3A5 (anti-CABYR) and anti-mouse Fab-cleavable-Duocarmycin complex for ˜72 hours. The presence of viable live cells per well was measured by total cellular ATP content in relative luminescence unit (RLU). Viability of control live cells (100%) was determined from an average of 9 wells in terms of total cellular ATP (in RLU) containing the constant 15 nm Fab-CL-DMDM for each set of antibody/reagent. The anti-SAS1B mAbs SB2, SB4, and SB5 killed 80%, 64%, 57% of cells, respectively, at 1 nM concentration when Fab-DMDM was kept constant at 15 nM for all primary antibody dilutions. SB2 killed 67% of cells at 0.1 nM concentration. The negative control, anti-CABYR mAb, 3A5, showed no killing at those concentrations. Staurosporine, a general protein kinase inhibitor, which induces apoptosis, was used as a positive control and DMSO was used as a negative control. The data are shown in FIG. 7A-B and represent an average of triplicate experiments.

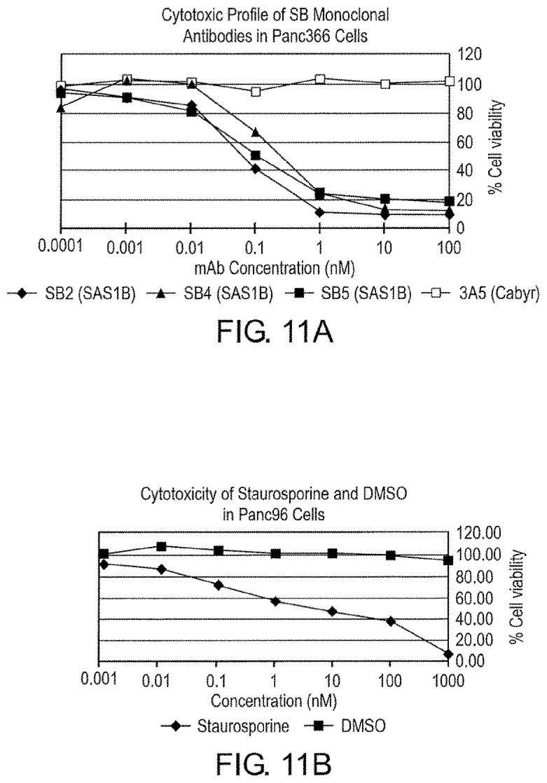

The cytotoxic effect of anti-human SAS1B mAbs SB2, SB4, SB5 on human pancreatic cancer cells (Panc366) were tested by ADC assay. Human pancreatic cancer cells 366 were seeded in triplicate on 96 well plates (1600 cells per well) overnight and then treated with SB2, SB4, SB5, and 3A5 (anti-CABYR) and anti-mouse Fab-cleavable-Duocarmycin complex for ˜72 hours. The presence of viable live cells per well was measured by total cellular ATP content in relative luminescence unit (RLU). Viability of control live cells (100%) was determined from an average of 9 wells in terms of total cellular ATP (in RLU) containing the constant 15 nm Fab-CL-DMDM for each set of antibody/reagent. SB2, SB4, and SB5 killed 88%, 77%, 76% of cells, respectively, at 1 nM concentration when Fab-DMDM was kept constant at 15 nM for all primary antibody dilutions. At 0.1 nM, SB2, SB4, and SB5 killed 59%, 33%, and 49% of cells, respectively. The negative control, 3A5, showed no killing at those concentrations. Staurosporine was used as a positive control and DMSO was used as a negative control. The data are shown in FIG. 11A-B and represents the average of triplicate experiments.

### Example 4 Pancreatic Cell Surface Staining by SAS1B Monoclonal Antibodies

Experiments were conducted to determine if SAS1B localizes to the cell surface of pancreatic cell lines (MPanc96 & 366), to identify which pancreatic cell line displays the most robust SAS1B cell surface staining, and to identify anti-SAS1B antibodies that show good cell surface staining. The live indirect immunofluorescence protocol is as follows. The cells were dissociated with Acutase and plated on fibronectin coated coverslips. MPanc96 cells were allowed to grow 2-3 days and 366 cells were allowed to grow for 3-4 days. The cells were blocked (blocking 1 solution) with 5% NGS in media for 30 min at RT and then blocked with 5% NGS in chilled media+0.1% sodium azide (blocking 2 solution) for 30 min on ice. The primary antibody (SB4, SB5 or control) was added at 20 ug/mL in blocking 2 solution for 2 hours on ice at 4° C. The cells were washed 3× for 5 minutes each on ice. The secondary antibody (goat anti-Ms/Rb Alexa488 (1:500)) was added in blocking 2 solution for 1 hour in dark on ice at 4° C. The cells were washed 3× for 5 min each on ice and then were fixed with 4% PFA-PBS for 15 min in the dark at RT. The cells were washed 2× for 5 min each at RT and then stained with DAPI stain (1:1000) for 10 min at RT. The cells were washed 2× for 5 min each at RT and then were treated with prolong gold antifade. SB4 showed the most abundant and robust cell surface staining of SAS1B for both MPanc96 and 366 (65-80% of cells staining). About 10-20% of the SB5 treated cells stained for SASB1.

### Example 5 Immunoprecipitatlon (“IP”) of SAS1B from HEK293T Cells Transfected with SAS1B

HEK293T cells were transfected with human SAS1B full length cDNA for 66 h. Immuno-precipitation of SAS1B was completed with SB1, SB2, SB4 and SB5 monoclonal antibodies. Following SAS1B immunoprecipitation, detection was completed with mouse anti-His tag mAb at C-terminus. Immunoprecipitated samples were analyzed by protein staining with Coomassie staining. Immunoprecipitated sample was also analyzed by MS/MS analysis. VH and VL chain CDR1-3 regions were sequenced for the confirmed anti-SAS1B mAbs. Immunoprecipitation was performed with N-terminal mAbs from transfected cells extracted with RIPA/protease inhibitor. Detection of the SAS1B immunoprecipitation product was completed with HRP peroxidase conjugated mouse anti-His mAb at 1:3000 or 1:5000 dilution. HEK293T cells were seeded at 250,000. The cells were transfected with 20 mg of SAS1B-SVA plasmid into each of the two T-75 flasks (at ˜90% confluency) for ˜66 h at 37 C and 5% CO₂. Following transfection, two saline washes were completed with 1×HBSS. Lysed cells were incubated with RIPA+protease inhibitor for one hour on ice with mixing to ensure homogenous lysate and supernatant used for IP (RIPA: 25 mM Tris-HCl (pH 7.6), 150 mM NaCl, 1% NP-40, 1% sodium deoxycholate, 0.1% SDS). For the immunoprecipitation, 3 mg magnetic bead coupled protein G were suspended and incubated with 20 mg (in 200 mL PBST) of each SAS1B monoclonal antibody for 10 minutes. The magnetic bead/antibody complex was washed with PBST once, followed by incubation with 300 mL antigen extract for 20 min. The post-immunoprecipitation sample was saved for comparison. Three 200 mL washes with washing buffer+protease inhibitor were completed. The fourth wash used only 100 mL of wash buffer+protease inhibitor. The magnetic beads were transferred to a new micro-centrifuge tube. 1× Lamelli buffer was added to bead/antibody/antigen complex for subsequent analysis. The sample was boiled at 98° C. for 10 min in 1× Lamelli buffer and the supernatant transferred to a new tube. The immunoprecipitation sample was analyzed by western blot and Coomassie blue staining. The SDS-PAGE samples (pre-IP, IP, and post IP extracts) were resolved with 12% TGX Criterion gels at 30 milliamps for 1 hr. Coomassie stain was added to determine the profile of IP samples. Following SDS-PAGE resolution, bands were transferred to nitrocellulose membrane at 1 Amp for 1 h at 5° C. The membrane was blocked with 5% dry milk/PBST, and incubated with mouse anti-His monoclonal antibody conjugated with HRP for 1 hr at RT at 1:3000 or 1:5000 dilution. The membranes were imaged with TMB solution.

The western blot showed the SAS1B antigen was pulled down by the monoclonal antibodies SB1 and SB5. The 3A7 mAb and protein G beads did not pull down SAS1B. The remaining SAS1B in the post IP (“PIP”) sample was compared with starting material (“SM”). SB1 and SB5 mAbs revealed relative depletion of antigen in post IP samples compared to starting material. SB2 and SB4 both pulled down recombinant SAS1B from HEK293T extract. The negative control mAb 3A7 did not pull down the antigen. Post IP (PIP) sample analysis revealed a clear relative reduction of SAS1B by SB4 compared to pre-IP starting material. 3A7 showed no reduction of SAS1B in post-IP samples.

The IP elutes of SB1, SB5, 3A7 and Blank Beads (BB) were analyzed. The 3A7 mAb and the blank beads were used as negative controls (SM, starting material). The heavy chain was stained at about 50 kD and the light chain stained at about 25 kD.

A unique ˜52 kD band was recovered in SB5 IP. SB5 (IgG2b) has a different isotype than SB1 (IgG2a), and 3A7 (IgG1). The ˜52 kD band from the SB5 immunoprecipitation was isolated, restained with Coomassie blue and submitted for MS/MS to verify protein identity. The MS/MS nanospray ion source was operated at 2.5 kV. The digest was analyzed using the rapid switching capability of the instrument acquiring a full screen mass spectrum to determine peptide molecular weights followed by product ion spectra to determine amino acid sequence in sequential scans. This mode of analysis produces about 20,000 MS/MS spectra of ions ranging in abundance over several orders of magnitude. Not all MS/MS spectra are derived from peptides. The data were analyzed using the Sequest search algorithm against Uniprot Human and SwissProt.

The band contained abundant peptides matching to human ASTL and Ig heavy chain (SwissProt). The only other proteins appear to be low abundance background proteins like keratins. A total of 12 unique human SAS1B/ASTL peptide sequences were recovered (33% of 431 amino acids) from SB5 IP of the ˜52 kD protein ranging from residue number 85 to 361 with multiple repeats. The MS/MS analysis of the SB5 IP sample further confirmed the specificity of the mAb for Human SAS1B protein.

### Example 6 Cytotoxicity Assays

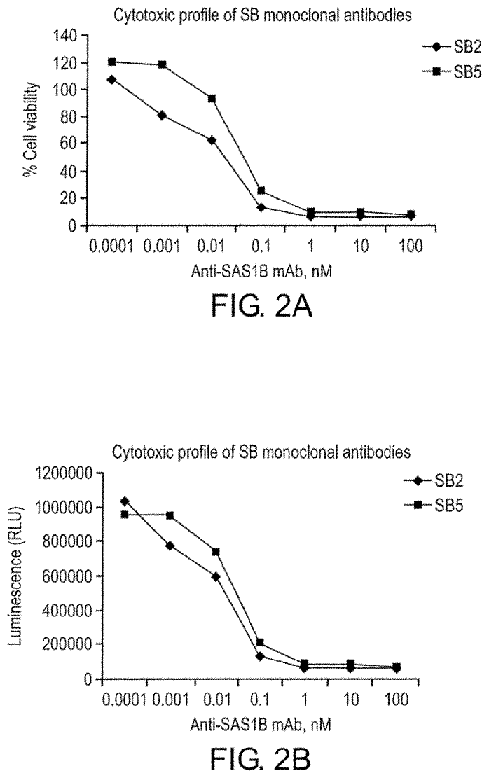

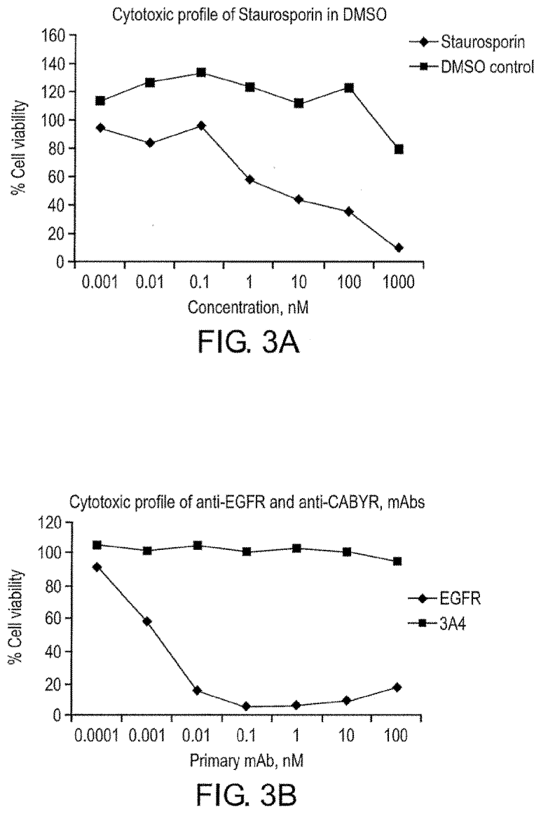

The cytotoxic effect of anti-human SAS1B mAbs SB2 and SB5 on human uterine cancer cells (MMMT539) was examined by antibody drug conjugate cytotoxicity (ADC) assay. Human uterine cancer cells MMMT539 were seeded in triplicate on 96 well plate overnight and then treated with SB2 or SB5 and anti-mouse Fab-cleavable-Duocarmycin complex for ˜90 h. The presence of viable live cells per well was measured by total cellular ATP content in relative luminescence unit (RLU). See FIG. 2B. Viability of control live cells (100%) was determined from average of 9 wells in terms of total cellular ATP (in RLU) containing the constant 15 nM Fab-CL-DMDM for each set of antibody/reagent. The SAS1B mAbs SB2 and SB5 killed 87% and 75% of the cancer cells at 0.1 nM concentration when Fab-DMDM was kept constant at 15 nM for all first antibody dilutions. See FIG. 2A. Staurosporine, a general protein kinase inhibitor, was used to induce apoptosis in 1% DMSO as a positive control for the assay. The anti-EGFR mAb and Staurosporine, both killed the cancer cells in a dose dependent manner. See FIG. 3A. The negative control anti-CABYR mAb, 3A4 showed no cell killing at those concentration of Fab-CL-DMDM. See FIG. 3B. The ATP standard curve showed linearity from 0 to 1000 nM. The cytotoxic effect confirms that these mAbs are clear therapeutic agents to target cancer cells that express SAS1B protein on the cell surface. The data represents an average from two experiments in triplicate.

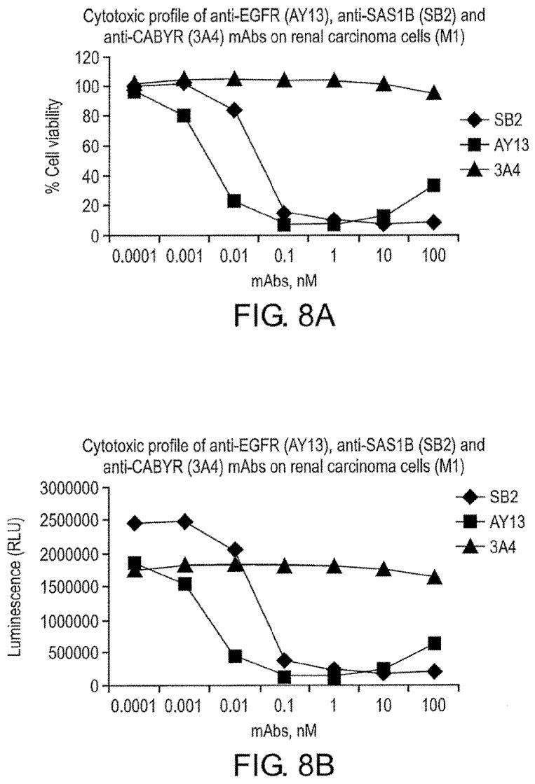

The cytotoxic effect of anti-EGFR (AY13), anti-SAS1B (SB2) and anti-CABYR (3A4) monoclonal antibodies on renal carcinoma cells were tested by ADC assay. An average of 3 experiments in triplicate is reported. Anti-CABYR mAb, 3A4 showed no cytotoxic effect at 15 nM Fab-DMDM for 4 days. Anti-SAS1B mAb, SB2 showed a cytotoxic effect at 0.01, 0.1 nM and higher concentrations. A higher cytotoxic effect was observed with EGFR mAb at 0.01 nM with only 20% viability compared to ˜80% viability for SB2 mAb. See FIGS. 8A-B.

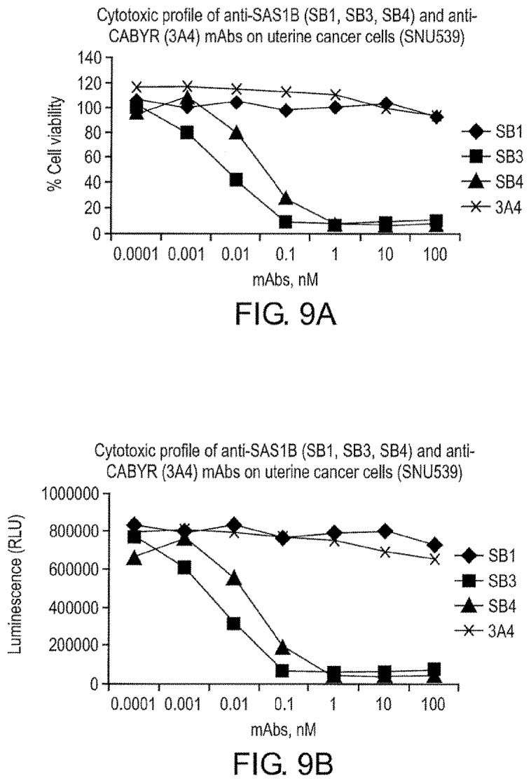

The cytotoxic effect of anti-human SAS1B monoclonal antibodies SB1, SB3 and SB4 on human uterine cancer cells was investigated by ADC assay using Fab-anti-mouse IgG Fc conjugated to Duocarmycin DM with a cleavable linker. An average of 3 experiments were done in triplicate. Anti-CABYR mAb, 3A4 and anti-SAS1B mAb SB1 showed no cytotoxic effect for 4 days. Anti-SAS1B mAb, SB4 showed cytotoxic effect at 0.01, 0.1 nM and higher concentrations. The strongest cytotoxic effect was observed with SB3 mAb. At 0.01 nM the viability was only 40% compared to ˜80% viability for SB4 mAb. See FIGS. 9A-B.

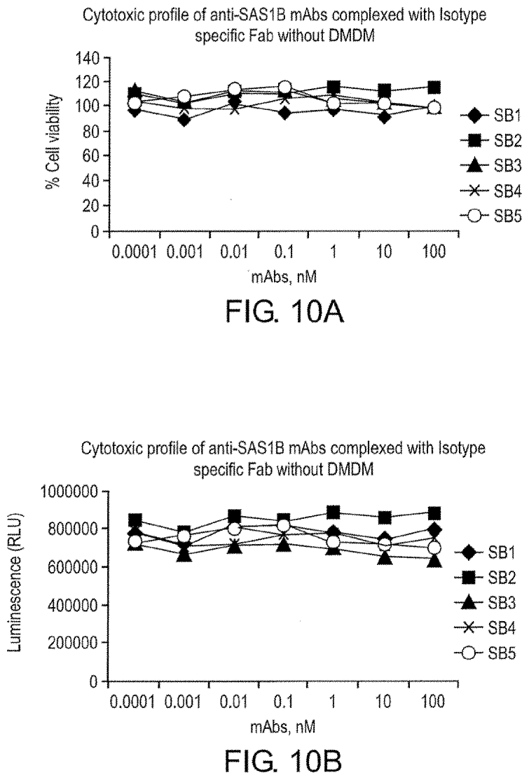

The cytotoxic effect of anti-human SAS1B mAbs SB1, SB2, SB3, SB4 and SB5 on human uterine cancer cells by ADC assay using unconjugated Fab-anti-mouse IgG Fc was examined. An average of 1 or 2 experiments in triplicate were performed. Anti-SAS1B mAbs, SB1, SB2, SB3, SB4 and SB5 mAbs showed no cytotoxic effect for 4 days in presence of isotype-specific unconjugated Fab. See FIGS. 10A-B. The cytotoxic effect of anti-human SAS1B mAbs SB1, SB2, SB3, SB4, and SB5 was tested on human uterine cancer cells by ADC assay using Fab-anti-mouse IgG Fc conjugated to Duocarmycin DM with cleavable linker. An average of 3 experiments were completed in triplicate. Anti-CABYR mAb, 3A4, and anti-SAS1B mAb SB1 showed no cytotoxic effect for 4 days. Anti-SAS1B mAbs, SB2, SB3, SB4 and SB5 showed strong cytotoxic effect at 0.1 nM and higher concentrations. The strongest cytotoxic effect was observed with SB3 mAb. At 0.01 nM the viability for SB3, SB2, SB4 and SB5 were 42%, 66%, 80% and 87% respectively.

The morphology of uterine cancer cell line (SNU539) and uterine cancer cell line (MMMT539) after 93 h of anti-SAS1B antibody drug treatment were examined. At 0.1 nM of SB2 or SB5 mAbs with 15 nM of Fab-DMDM, a large number of rounded dead cells and few live cells were observed compared to very few dead cells at 0 nM control. Both SB2 and SB5 mAbs showed clear cell killing and inhibition of cell proliferation.

The morphology of renal cell carcinoma cell line (M1) after 93 h of anti-EGFR antibody drug treatment was examined. At 0.1 nM of anti-EGFR mAb with 15 nM of Fab-DMDM, the numbers of unrounded live cells were very few compared to 0 nM control. The control mAb, 3A4 (anti-CABYR) showed no cell killing and no inhibition of cell proliferation unlike anti-EGFR mAb. At 1 nM of SB2 or anti-EGFR mAbs with 15 nM of Fab-DMDM, a large number of rounded dead cells were observed compared to very few rounded dead cells at 0 nM control. Both SB5 and EGFR mAbs showed clear cell killing and inhibition of cell proliferation properties at 1 nM conc.

### Example 7 Development of an ELISA Assay

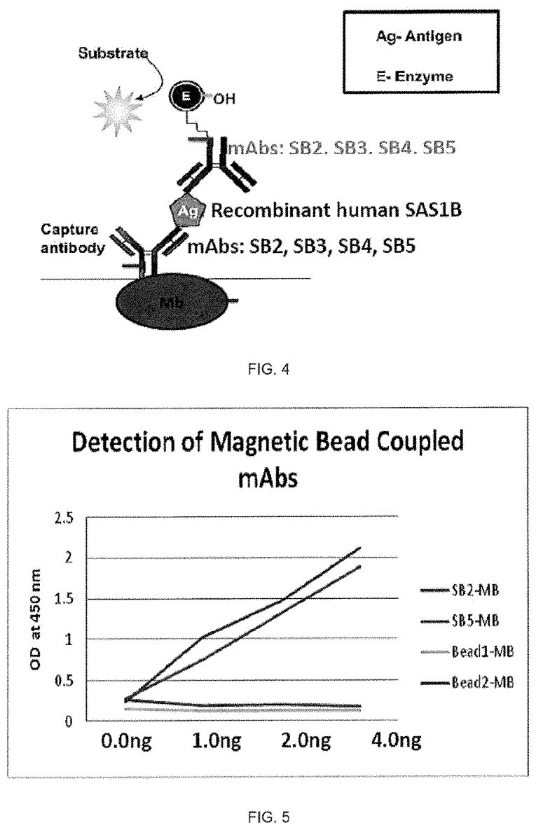

The antibodies were used to develop a capture assay by sandwich ELISA. See FIG. 4. The support was a magnetic bead (MB) coupled to SB2, SB3, SB4, or SB5 mAbs. The antigen was a recombinant human SAS1B antigen. Detection was accomplished with mAbs SB2, SB3, SB4, SB5 conjugated with HRP. A HRP substrate was used and TMB was used for quantitation.

In one experiment magnetic beads were coupled to anti-human SAS1B monoclonal antibodies SB2 and SB5 IgGs. The assay method captured 6.7 femtomole of magnetic bead coupled IgGs. See FIG. 5.

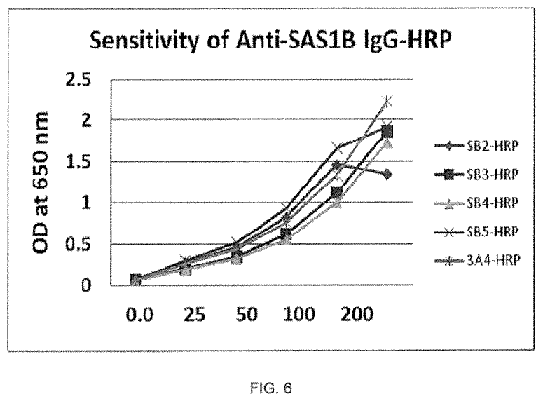

The retention of HRP activity after conjugations to SASB1 SB2, SB3, SB4, SB5, and anti-CABYR monoclonal antibody 3A4 was examined. The assay method detected 0.2 femtomole of IgGs. See FIG. 6. Where SB2 is coupled to magnetic beads and SB5 coupled to HRP is used as a detection agent in a 96 well ELISA format 2.7 pmole/ml of rhSAS1B can be detected.

### Example 8 Sequences

Total RNA was extracted from SB1, SB2, SB3, SB4, SB5, SB6, and SB7 hybridoma cells. RACE (Rapid Amplification of cDNA Ends) was performed to amplify DNA for V_{H} and V_{L}. Positive clones were identified by gel electrophoresis. The positive DNA was TOPO cloned and sequenced and the DNA and amino acid sequences for V_{H}, V_{L} and CDRs were identified using VBASE2.

SB1

CDR1 of the SB1 heavy chain is GYTFTDYN (SEQ ID NO:1). CDR2 of the SB1 heavy chain is INPNNGGT (SEQ ID NO:2). CDR3 of the SB1 heavy chain is ATNEY (SEQ ID NO:3). CDR1 of the SB1 light chain is ENVGTY (SEQ ID NO:4). CDR2 of the SB1 light chain is GAS. CDR3 of the SB1 light chain is GQSYSYPWT (SEQ ID NO:5). The amino acid sequence of the variable heavy chain of SB1 is:

(SEQ ID NO: 12)
EVQLQQSGPELVKPGSSVKISCKASGYTFTDYNMDWVKQSHGKSLEWIGAI
NPNNGGTSYNQKFKGKATLTVDKSSSTAYMELRSLTSEDSAVYYCATNEYW
GQGTTLTVSS

The nucleotide sequence of the SB1 variable heavy chain is:

(SEQ ID NO: 19)
GAGGTCCAGCTGCAACAGTCTGGACCTGAGCTGGTGAAGCCTGGGTCTTCA
GTGAAGATATCCTGCAAAGCTTCTGGATACACATTCACTGACTACAACATG
GACTGGGTGAAGCAGAGCCATGGAAAGAGCCTTGAGTGGATTGGAGCTATT
AATCCTAACAATGGTGGTACTAGCTACAACCAGAAGTTCAAGGGCAAGGCC
ACATTGACTGTAGACAAGTCCTCCAGTACAGCCTACATGGAGCTCCGCAGC
CTGACATCTGAGGACTCTGCAGTCTATTACTGTGCAACAAATGAGTACTGG
GGCCAAGGCACCACTCTCACAGTCTCCTCA

The amino acid sequence of the SB1 variable light chain is:

(SEQ ID NO: 13)
NIVMTQSPKSMSMSVGERVTLSCKASENVGTYVSWYQQKPEQSPKLLIYGA
SNRYTGVPDRFTGSGSATDFTLTIS SVQAEDLADYHCGQSYSYPWTFGGG
TKLEIK

The nucleotide sequence of the SB1 variable light chain is:

(SEQ ID NO: 20)
AACATTGTAATGACCCAATCTCCCAAATCCATGTCCATGTCAGTAGGAGAG
AGGGTCACCTTGAGCTGCAAGGCCAGTGAGAATGTGGGTACTTATGTATCC
TGGTATCAACAGAAACCAGAGCAGTCTCCTAAACTGCTGATATACGGGGCA
TCCAACCGGTACACTGGGGTCCCCGATCGCTTCACAGGCAGTGGATCTGCA
ACAGATTTCACTCTGACCATCAGCAGTGTGCAGGCTGAAGACCTTGCAGAT
TATCACTGTGGACAGAGTTACAGCTATCCGTGGACGTTCGGTGGAGGCACC
AAGCTGGAAATCAAA

SB2

CDR1 of the SB2 variable heavy chain is GYTFTDYN (SEQ ID NO:1). CDR2 of the SB2 variable heavy chain is VNPNNGGT (SEQ ID NO:6). CR3 of the SB3 variable heavy chain is VPNWDWFAY (SEQ ID NO:7). CDR1 of the SB2 variable light chain is QSLVHSNGNTY (SEQ ID NO:8). CDR2 of the SB2 variable light chain is KVS. CDR3 of the SB2 variable light chain is FQGSHVPFT (SEQ ID NO:9).

The amino acid sequence of the SB2 variable heavy chain is:

(SEQ ID NO: 14)
EVQLQQSGPELVKPGSSVKISCKASGYTFTDYNMVWVKQSHGKSLEWIGAV
NPNNGGTSYNQKFKGKATLTVDKSSSTAYMELRSLTSEDSAVYYCVPNWDW
FAYWGQGTLVTVSA

The nucleotide sequence of the SB2 variable heavy chain is:

(SEQ ID NO: 21)
GAGGTCCAGCTGCAACAGTCTGGACCTGAGCTGGTGAAGCCTGGGTCTTCA
GTGAAGATATCCTGCAAAGCTTCTGGATACACATTCACTGACTACAACATG
GTCTGGGTGAAGCAGAGCCATGGAAAGAGCCTTGAGTGGATTGGAGCTGTT
AATCCTAACAATGGTGGTACTAGCTACAACCAGAAGTTCAAGGGCAAGGCC
ACATTGACTGTAGACAAGTCCTCCAGCACAGCCTACATGGAGCTCCGCAGC
CTGACATCTGAGGACTCTGCAGTCTATTACTGTGTCCCCAACTGGGACTGG
TTTGCTTACTGGGGCCAAGGGACTCTGGTCACTGTCTCTGCA

The amino acid sequence of the SB2 variable light chain is:

(SEQ ID NO: 15)
DVLMTQTPLSLPVSLGDQASISCRSSQSLVHSNGNTYLEWYLQKPGQSPNL
LIYKVSNRFSGVPDRFSGSGSGTDF TLKISRVEAEDLGVYYCFQGSHVPF
TFGSGTKLEIK

The nucleic acid sequence of the SB32 variable light chain is:

(SEQ ID NO: 22)
GATGTTTTGATGACCCAAACTCCACTCTCCCTGCCTGTCAGTCTTGGAGAT
CAAGCCTCCATCTCTTGCAGATCTAGTCAGAGCCTTGTACATAGTAATGGA
AACACCTATTTAGAATGGTACCTGCAGAAACCAGGCCAGTCTCCAAACCTC
CTGATCTACAAAGTTTCCAACCGATTTTCTGGGGTCCCAGACAGGTTCAGT
GGCAGTGGATCAGGGACAGATTTCACACTCAAGATCAGCAGAGTGGAGGCT
GAGGATCTGGGAGTTTATTACTGCTTTCAAGGTTCACATGTTCCATTCACG
TTCGGCTCGGGGACAAAGTTGGAAATAAAA

SB3

CDR1 of the SB3 heavy chain is GYTFTDYN (SEQ ID NO:1). CDR2 of the SB3 heavy chain is VNPNNGGT (SEQ ID NO:6). CDR3 of the SB3 heavy chain is VPNWDWFAY (SEQ ID NO:7). CDR1 of the SB3 light chain is QSLVHSNGNTY (SEQ ID NO:8). CDR2 of the SB3 light chain is KVS. CDR3 of the SB3 light chain is FQGSHVPFT (SEQ ID NO:9).

The amino acid sequence of the SB3 variable heavy chain is:

(SEQ ID NO: 14)
EVQLQQSGPELVKPGSSVKISCKASGYTFTDYNMVWVKQSHGKSLEWIGAV
NPNNGGTSYNQKFKGKATLTVDKSS STAYMELRSLTSEDSAVYYCVPNWD
WFAYWGQGTLVTVSA

The nucleic acid sequence of the SB3 variable heavy chain is:

(SEQ ID NO: 21)
GAGGTCCAGCTGCAACAGTCTGGACCTGAGCTGGTGAAGCCTGGGTCTTCA
GTGAAGATATCCTGCAAAGCTTCTGGATACACATTCACTGACTACAACATG
GTCTGGGTGAAGCAGAGCCATGGAAAGAGCCTTGAGTGGATTGGAGCTGTT
AATCCTAACAATGGTGGTACTAGCTACAACCAGAAGTTCAAGGGCAAGGCC
ACATTGACTGTAGACAAGTCCTCCAGCACAGCCTACATGGAGCTCCGCAGC
CTGACATCTGAGGACTCTGCAGTCTATTACTGTGTCCCCAACTGGGACTGG
TTTGCTTACTGGGGCCAAGGGACTCTGGTCACTGTCTCTGCA

The amino acid sequence of the SB3 variable light chain is:

(SEQ ID NO: 15)
DVLMTQTPLSLPVSLGDQASISCRSSQSLVHSNGNTYLEWYLQKPGQSPNL
LIYKVSNRFSGVPDRFSGSGSGTDFTLKISRVEAEDLGVYYCFQGSHVPFT
FGSGTKLEIK

The nucleic acid sequence of the SB3 variable light chain is:

(SEQ ID NO: 22)
GATGTTTTGATGACCCAAACTCCACTCTCCCTGCCTGTCAGTCTTGGAGAT
CAAGCCTCCATCTCTTGCAGATCTAGTCAGAGCCTTGTACATAGTAATGGA
AACACCTATTTGGAATGGTACCTGCAGAAACCAGGCCAGTCTCCAAACCTC
CTGATCTACAAAGTTTCCAACCGATTTTCTGGGGTCCCAGACAGGTTCAGT
GGCAGTGGATCAGGGACAGATTTCACACTCAAGATCAGCAGAGTGGAGGCT
GAGGATCTGGGAGTTTATTACTGCTTTCAAGGTTCACATGTTCCATTCACG
TTCGGCTCGGGGACAAAGTTGGAAATAAAA

SB4

CDR of the heavy chain variable region of SB4 is GYTFTDYN (SEQ ID NO:1). CDR2 of the heavy chain variable region of SB4 is INPNNGGT (SEQ ID NO:2). CDR3 of the heavy chain variable region of SB4 is APNWDWFAY (SEQ ID NO:10). CDR1 of the light chain variable region of SB4 is QSILHSNGNTY (SEQ ID NO:11). CDR2 of light chain variable region is KVS. CDR3 of the light chain variable region of SB4 is FQGSHVPFT (SEQ ID NO:9).

The amino acid sequence of the heavy chain variable region of SB4 is as follows:

(SEQ ID NO: 16)
EVQLQQSGPELVKPGSSVKISCKPSGYTFTDYNMVWMKQSHGKSLEWIGAI
NPNNGGTTYNQKFKGKATLTVDKSSSTAYMELRSLTSEDSAVYYCAPNWDW
FAYWGQGTLVTVSA

The nucleotide sequence of the heavy chain variable region of SB4 is as follows:

(SEQ ID NO: 23)
GAGGTCCAGCTGCAACAGTCTGGACCTGAGCTGGTGAAGCCTGGGTCTTC
AGTGAAGATATCCTGCAAACCTTCTGGATACACATTCACTGACTACAACA
TGGTCTGGATGAAGCAGAGCCATGGAAAGAGCCTTGAGTGGATTGGAGCT
ATTAATCCTAACAATGGTGGTACTACCTACAACCAGAAGTTCAAGGGCAA
GGCCACATTGACTGTAGACAAGTCCTCCAGCACAGCCTACATGGAGCTCC
GCAGCCTGACATCTGAGGACTCTGCAGTCTATTACTGTGCCCCCAACTGG
GACTGGTTTGCTTACTGGGGCCAAGGGACTCTGGTCACTGTCTCTGCA

The amino acid sequence of the light chain variable region of SB4 is as follows:

(SEQ ID NO: 17)
DVLMTQTPLSLPVSLGDQASISCRSSQSILHSNGNTYLEWYLQKPGQSPK
LLIYKVSNRFSGVPDRFSGSGSGTDFTLKISRVEAEDLGVYYCFQGSHVP
FTFGSGTKLEIK

The nucleotide sequence of the light chain variable region of SB4 is as follows:

(SEQ ID NO: 24)
GATGTTTTGATGACCCAAACTCCACTCTCCCTGCCTGTCAGTCTTGGAGA
TCAAGCCTCCATCTCTTGCAGATCTAGTCAGAGCATTTTACATAGTAATG
GAAACACCTATTTAGAATGGTACCTGCAGAAACCAGGCCAGTCTCCAAAG
CTCCTGATCTACAAAGTTTCCAACCGATTTTCCGGGGTCCCAGACAGGTT
CAGTGGCAGTGGATCAGGGACAGATTTCACACTCAAGATCAGCAGAGTGG
AGGCTGAGGATCTGGGAGTTTATTACTGCTTTCAAGGTTCACATGTTCCA
TTCACGTTCGGCTCGGGGACAAAGTTGGAAATAAAAC

The isotype for SB4 is IgG2a-k.

SB5

CDR1 of the heavy chain variable region of SB5 is GYTFTDYN (SEQ ID NO:1). CDR2 of the heavy chain variable region of SB5 is INPNNGGT (SEQ ID NO:2). CDR3 of the heavy chain region of SB5 is APNWDWFAY (SEQ ID NO:10). CDR1 of the light chain variable region of SB5 is QSILHSNGNTY (SEQ ID NO:11). CDR2 of the light chain variable region of SB5 is KVS. CDR3 of the light chain variable region of SB5 is FQGSHVPFT (SEQ ID NO:9).

The amino acid sequence of the heavy chain variable region of SB5 is as follows:

(SEQ ID NO: 16)
EVQLQQSGPELVKPGSSVKISCKPSGYTFTDYNMVWMKQSHGKSLEWIGA
INPNNGGTTYNQKFKGKATLTVDKSSSTAYMELRSLTSEDSAVYYCAPNW
DWFAYWGQGTLVTVSA

The nucleotide sequence of the heavy chain variable region of SB5 is as follows:

(SEQ ID NO: 23)
GAGGTCCAGCTGCAACAGTCTGGACCTGAGCTGGTGAAGCCTGGGTCTTC
AGTGAAGATATCCTGCAAACCTTCTGGATACACATTCACTGACTACAACA
TGGTCTGGATGAAGCAGAGCCATGGAAAGAGCCTTGAGTGGATTGGAGCT
ATTAATCCTAACAATGGTGGTACTACCTACAACCAGAAGTTCAAGGGCAA
GGCCACATTGACTGTAGACAAGTCCTCCAGCACAGCCTACATGGAGCTCC
GCAGCCTGACATCTGAGGACTCTGCAGTCTATTACTGTGCCCCCAACTGG
GACTGGTTTGCTTACTGGGGCCAAGGGACTCTGGTCACTGTCTCTGCA

The amino acid sequence of the light chain variable region of SB5 is as follows:

(SEQ ID NO: 18)
DVLMTQIPLSLPVSLGDQASISCRSSQSILHSNGNTYLEWYLQKPGQSPK
LLIYKVSNRFSGVPDRFSGSGSGTDFTLKISRVEAEDLGVYYCFQGSHVP
FTFGSGTKLEIK

The nucleotide sequence of the light chain variable region of SB5 is as follows:

(SEQ ID NO: 25)
GATGTTTTGATGACCCAAATTCCACTCTCCCTGCCTGTCAGTCTTGGAGA
TCAAGCCTCCATCTCTTGCAGATCTAGTCAGAGCATTTTACATAGTAATG
GAAACACCTATTTAGAATGGTACCTGCAGAAACCAGGCCAGTCTCCAAAG
CTCCTGATCTACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTACTGCTTTCAAGGTTCACATGTTCCA
TTCACGTTCGGCTCGGGGACAAAGTTGGAAATAAAAC

The isotype for SB5 is IgG2b-k.

SB6

CDR1 of the SB6 variable heavy chain is GYTFTDYN (SEQ ID NO:1). CDR2 of the SB6 variable heavy chain is INPNNGGT (SEQ ID NO:2). CDR3 of the SB6 variable heavy chain is APNWDWFAY (SEQ ID NO:10). CDR1 of the SB6 variable light chain QSILHSNGNTY (SEQ ID NO:11). CDR2 of the variable light chain of SB6 is KVS. CDR3 of the SB6 variable light chain is FQGSHVPFT (SEQ ID NO:9).

The amino acid sequence of the SB6 variable heavy chain is:

(SEQ ID NO: 16)
EVQLQQSGPELVKPGSSVKISCKPSGYTFTDYNMVWMKQSHGKSLEWIGA
INPNNGGTTYNQKFKGKATLTVDKSS STAYMELRSLTSEDSAVYYCAPN
WDWFAYWGQGTLVTVSA

The nucleic acid sequence of the SB6 variable heavy chain is:

(SEQ ID NO: 23)
GAGGTCCAGCTGCAACAGTCTGGACCTGAGCTGGTGAAGCCTGGGTCTTC
AGTGAAGATATCCTGCAAACCTTCTGGATACACATTCACTGACTACAACA
TGGTCTGGATGAAGCAGAGCCATGGAAAGAGCCTTGAGTGGATTGGAGCT
ATTAATCCTAACAATGGTGGTACTACCTACAACCAGAAGTTCAAGGGCAA
GGCCACATTGACTGTAGACAAGTCCTCCAGCACAGCCTACATGGAGCTCC
GCAGCCTGACATCTGAGGACTCTGCAGTCTATTACTGTGCCCCCAACTGG
GACTGGTTTGCTTACTGGGGCCAAGGGACTCTGGTCACTGTCTCTGCA

The amino acid sequence of the SB36 variable light chain is:

(SEQ ID NO: 26)
DVLMTQIPLSLPVSLGDQASISCRSSQSILHSNGNTYLEWYLQKPGQSPK
LLIYKVSNRFSGVPDRFSGSGSGTDFTLKISRVEAEDLGVYYCFQGSHVP
FTFGSGTKLEIK

The nucleic acid sequence of the SB6 variable light chain is:

(SEQ ID NO: 18)
GATGTTTTGATGACCCAAATTCCACTCTCCCTGCCTGTCAGTCTTGGAGA
TCAAGCCTCCATCTCTTGCAGATCTAGTCAGAGCATTTTACATAGTAATG
GAAACACCTATTTAGAATGGTACCTGCAGAAACCAGGCCAGTCTCCAAAG
CTCCTGATCTACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTACTGCTTTCAAGGTTCACATGTTCCA
TTCACGTTCGGCTCGGGGACAAAGTTGGAAATAAAA

SB7

CDR1 of the SB7 variable heavy chain is GYTFTDYN (SEQ ID NO:1). CDR2 of the SB7 variable heavy chain is INPNNGGT (SEQ ID NO:2). CDR3 of the SB7 variable heavy chain is APNWDWFAY (SEQ ID NO:10). CDR1 of the SB7 variable light chain is QSILHSNGNTY (SEQ ID NO:11). CDR2 of the variable light chain is KVS. CDR3 of the variable light chain is FQGSHVPFT (SEQ ID NO:9).

The amino acid sequence of the SB7 variable heavy chain is:

(SEQ ID NO: 16)
EVQLQQSGPELVKPGSSVKISCKPSGYTFTDYNMVWMKQSHGKSLEWIGA
INPNNGGTTYNQKFKGKATLTVDKSSSTAYMELRSLTSEDSAVYYCAPNW
DWFAYWGQGTLVTVSA

The nucleic acid sequence of the SB7 variable heavy chain is:

(SEQ ID NO: 23)
GAGGTCCAGCTGCAACAGTCTGGACCTGAGCTGGTGAAGCCTGGGTCTTC
AGTGAAGATATCCTGCAAACCTTCTGGATACACATTCACTGACTACAACA
TGGTCTGGATGAAGCAGAGCCATGGAAAGAGCCTTGAGTGGATTGGAGCT
ATTAATCCTAACAATGGTGGTACTACCTACAACCAGAAGTTCAAGGGCAA
GGCCACATTGACTGTAGACAAGTCCTCCAGCACAGCCTACATGGAGCTCC
GCAGCCTGACATCTGAGGACTCTGCAGTCTATTACTGTGCCCCCAACTGG
GACTGGTTTGCTTACTGGGGCCAAGGGACTCTGGTCACTGTCTCTGCA

The amino acid sequence of the SB7 variable light chain is:

(SEQ ID NO: 18)
DVLMTQIPLSLPVSLGDQASISCRSSQSILHSNGNTYLEWYLQKPGQSPK
LLIYKVSNRFSGVPDRFSGSGSGTDFTLKISRVEAEDLGVYYCFQGSHVP
FTFGSGTKLEIK

The nucleic acid sequence of the SB7 variable light chain is:

(SEQ ID NO: 25)
GATGTTTTGATGACCCAAATTCCACTCTCCCTGCCTGTCAGTCTTGGAGA
TCAAGCCTCCATCTCTTGCAGATCTAGTCAGAGCATTTTACATAGTAATG
GAAACACCTATTTAGAATGGTACCTGCAGAAACCAGGCCAGTCTCCAAAG
CTCCTGATCTACAAAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTACTGCTTTCAAGGTTCACATGTTCCA
TTCACGTTCGGCTCGGGGACAAAGTTGGAAATAAAA

### Compositions and Methods for Detecting and Treating Cancer

A. Significance.

New compositions and methods for diagnosing and treating cancer are disclosed herein which impact the co-development of SAS1B diagnostics and SAS1B-directed immunotherapeutics and lay the groundwork for a therapeutic monoclonal antibody-drug conjugate (ADC) and/or T-cell immunotherapy that targets the specific and unique cell surface epitopes of the SAS1B metalloprotease. RNA- and protein-based companion diagnostic tests for SAS1B are developed for use on biopsies or tumor specimens in order to identify patients who will potentially benefit from SAS1B targeted therapy.

The aims generated data on the topology of the SAS1B molecule at tumor cell surfaces by defining alternative splice variants that function as integral membrane proteins and by mapping surface accessible epitopes recognized by mAbs. The experiments developed quantitative PCR assays and monoclonal antibody probes suitable for studying the range of ASTL transcripts and SAS1B proteins in tumor specimens, determined the specific SAS1B isoforms and ectodomains accessible at tumor cell surfaces, selected mAbs with tumoricidal properties in vitro, defined SAS1B alternative splice variants predominating in tumors, and explored the incidence of SAS1B expression in different types of ovarian and uterine cancers (OUCs). Achievement of these aims is basic to co-development of immunotherapies that target SAS1B along with companion assays that diagnose SAS1B positive (SAS1B+) tumors and enrich patient populations for SAS1B targeted treatments.

A.1 Better Diagnosis and Therapy of Ovarian and Uterine Cancer.

The incidence of ovarian cancer (OC) has been increasing. In 2014, an estimated 21,980 new cases and 14,270 deaths from OC in the US were predicted. OC has a higher fatality-to-case ratio than any other gynecologic malignancy. It is the most deadly gynecologic malignancy in developed countries largely due to a lack of known risk factors or predictive bio-markers. This high mortality rate results in large part because the majority of women are not diagnosed until the disease is in an advanced, metastatic stage. 75% of OC patients first present to oncologists with International Federation of Gynecology and Obstetrics (FIGO) criteria stage III or IV disease, i.e. with metastases in the abdomen, frequently within the mesenteries suspending the bowel, or at sites such as the liver. For these patients, the 5-year survival rates are <30%. In contrast, the small proportion of patients diagnosed with stage I disease have a 5-year survival rate of >90%. More effective therapies are urgently needed for OC, especially for patients whose disease has disseminated. With respect to uterine cancer, the American Cancer Society predicted 52,630 new cases of uterine cancer and 8,590 deaths in the US in 2014. The majority of these cases will be endometrial adenocarcinomas, which are generally slow growing (Type 1). Uterine cancers such as Malignant Mullerian Mixed Tumors (MMMTs), having a mixture of epithelial and mesenchymal cell types, are aggressive (Type 2) and occur most frequently after menopause. Current therapeutics for ovarian and uterine cancer (OUC) target molecules such as DNA, microtubules, and cell surface receptors present in normal healthy tissues in addition to tumor cells, resulting in unwanted side-effects. The therapeutic target of this program, SAS1B, is a tumor-restricted cell surface enzyme that has the potential to limit the scope of unwanted drug side effects in OUC therapy due to its biological properties, tissue-specific distribution pattern, and prevalence in ovarian and uterine cancers.

A.2 Literature and Bioinformatics on the ASTL Gene and the SAS1B Therapeutic Target [TT].

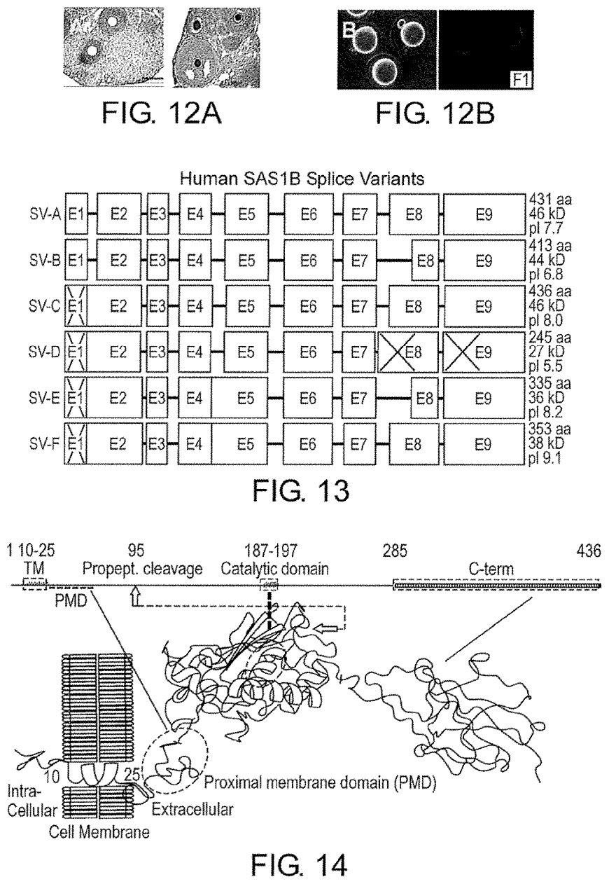

The ASTL (astacin-like) gene and its encoded SAS1B protein in mice and man has been previously reported, as well as reports on gene chip expression profiling of ASTL levels in various cell types. ASTL mRNAs has been demonstrated by in-situ hybridization only in oocytes and it has been proposed that SAS1B functioned as a hatching enzyme at the time the blastocyst emerges from the zona pellucida, based on structural similarities of SAS1B to other matrix metalloproteases (MMPs) known to be involved in hatching, specifically the astacins. Subsequently, SAS1B protein was shown to be present during mouse oogenesis only in oocytes, beginning during the development of secondary follicles. Ovaries from several Eutherian orders including primates showed translation of SAS1B only in growing oocytes from secondary follicle stages onward and an absence of SAS1B expression in the ovarian reserve of oocytes in primordial and primary follicles. SAS1B proteins persisted in the first few cleavage stages of the early embryo but were absent by the time murine embryo reached blastocyst stage, suggesting that a function for SAS1B in blastocyst hatching was unlikely. FIG. 12A (right panel) shows mouse oocytes stained dark red-brown with an antibody to SAS1B. Only the oocytes (right panel) stain with the antibody to SAS1B, while other ovarian tissues lack staining. No staining with preimmune sera was noted (see FIG. 12A (left panel)). In live unpermeabilized ovulated oocytes, SAS1B localized on the plasma membrane overlying a specific region called the microvillar domain (see FIG. 12B). This is the region of the oocyte membrane that interacts with sperm and is where fertilization occurs. It has been demonstrated that recombinant SAS1B cleaves one of the zona pellucida proteins (ZP2) leading to the postulate that SAS1B plays a role in the block to sperm binding after fertilization. Targeted deletions of ASTL in mice by two groups both resulted in sub-fertile phenotypes. The demonstration by the PI's group of 6 alternative astl splice variants, blockage of in-vitro fertilization by anti-SAS1B antibodies, cell surface expression of SAS1B in transfected CHO cells, and evidence showing interaction of SAS1B with an intraacrosomal protein SLLP1, led to the conclusion that SAS1B is involved in sperm-egg interaction, with SAS1B serving as an oolemmal receptor for SLLP1. These attributes led to the protein designation: Sperm Acrosomal SLLP1 Binding protein. Genbank EST (expressed sequence tag) deposits in mice show astl expression among normal tissues only in the ovary, oocytes, and in blastomeres of the first few cleavage stages. This EST data agrees with mRNA levels by Northern blot and gene profiling showing SAS1B is 5-fold downregulated in 1 cell mouse embryos compared to oocytes.

In humans, ASTL has been detected at the message level in the ovulated human oocyte. SAS1B protein has been detected only in growing oocytes in sections of human ovaries and localized on the oocyte plasma membrane in ovulated oocytes. SAS1B mRNAs has been identified in two human ovarian carcinomas. Genbank EST deposits in humans show a single hit for ASTL in the uterus and this deposit is from a uterine cancer specimen. The PI's lab has studied a pilot cohort of Type 1 & 2 uterine cancers (N=75) and estimated the incidence of SAS1B expression at 77%. Uterine cancer cell lines and tumor cells extracted from primary tumors show SAS1B localization at the cell surface and complexes of polyclonal antibodies with SAS1B internalize into endosomes within 15 minutes of antibody binding at the cell surface and then enter the lysosomal compartment in uterine cancer cells. Moreover, SAS1B-directed immunotoxins with cathepsin cleavable linker arms arrest cancer cell growth in vitro. In sum, basic reproductive biology literature on ASTL transcription and the localization of SAS1B proteins indicate that, among normal adult tissues, the SAS1B metalloprotease is selectively expressed only in the ovary in the pool of growing oocytes. The tumor biology literature indicates that SAS1B is accessible to antibody binding at tumor cell surfaces. Antibody-SAS1B complexes internalize into the endo-lysosomal system and SAS1B internalization can serve as a means to deliver and release drug payloads inside tumor cells. Based on preliminary studies by the PI's lab, ovarian and uterine tumors express SAS1B at high frequency.

B. Innovation.

B1: SAS1B is a Target for Tumor-Selective Therapy.

Due largely to the lack of specificity in their mechanisms of action, current chemotherapies and targeted therapies for OUC may result in adverse drug reactions including short-term and reversible conditions such as nausea, diarrhea, hair loss, and anemia, as well as longer-term permanent effects such as infertility and neuropathy. DNA, RNA, and microtubules represent molecular targets of current therapeutics that function in central cellular processes and pathways in all cells. Because of the unique oocyte-restricted pattern of expression of the SAS1B drug target among normal adult tissues, SAS1B differs from other drug targets that have been previously discovered and are now utilized for cancer treatments. The receptors (estrogen receptor, EGFR, VEGF, and Her2/neu), previously chosen as drug targets for development of ITs, are elevated in tumors, but are present in other normal tissues. Thus, the current drug targets show only a quantitative difference in their expression patterns between the tumor and healthy tissues.

In contrast, the SAS1B drug target has a qualitative and absolute expression difference between the tumor and normal healthy tissues (with the exception of its expression in the pool of growing oocytes). Thus, the creation of a new IT to SAS1B has the potential to minimize unwanted side effects, including infertility, and could become a front-line targeted therapy for OUC. By defining the ASTL splice variants present in cancers, defining which splice variants encode integral membrane isoforms of SAS1B, and perfecting qPCR methods to quantitate their relative abundance optimal diagnostic assays will be developed that can identify patient populations who will be helped by a SAS1B-targeted therapy. By identifying mAbs with tumoricidal activity that map to surface exposed epitopes on SAS1B, lead drug candidates can be defined. mAbs can humanized for testing as antibody-drug conjugates or “naked” antibodies, and ScFvs can be created that can later be engineered as chimeric antigen receptors (CAR-T) or transduced T-cell receptors.

B.2 Opening a New Field of Cancer-Oocyte Neoantigens.

During the past 20 years, a broad field of molecular cancer immunology emerged known as cancer-testis antigens (CTAs). The protein molecules that fulfill the criteria of CTAs are usually restricted to the testis among normal tissues, being found only in the germ cells, with expression at various steps during normal spermatogenesis. CTAs have found clinical use in cancer vaccines (e.g., Mage-A3 in the MARGIT and DERMA trials in lung and melanoma cancers) and for adoptive transfer of T cell receptor transduced T cells (e.g., NY-ESO-1 in melanoma and synovial cell sarcoma). The Cancer Research Institute of New York maintains a comprehensive database of known CTAs. Although the CTA field has developed several immunotherapeutic targets for clinical use, little is presently known about the role of oocyte-specific proteins in cancers. The neologism, canceroocyte neoantigens delineates a promising new field defined as gene products expressed widely in tumors that are expressed among normal tissues only in the ovary at precise stages of oocyte growth and follicular maturation. It has long been appreciated that tumors may de-differentiate, express genes typical of earlier developmental stages and revert to phenotypes reminiscent of embryonic development. However, little attention has been given to tumor markers and drug targets that are specific to the original stem cell, the oocyte.

C. Approach

C.1 Preliminary Results.

The first report on the subject of this application named the astacin-like gene (ASTL) based on homologous sequences for the crawfish and other astacins that had been found previously in oocytes. However, bioinformatic alignments (GenBank) of the human ASTL amino acid sequences currently show higher homologies (percent overlap and identical amino acids) with the meprin zinc metalloproteases, a group with known membrane isoforms, than with the astacin group of hatching metalloproteases (alignments not shown). These alignments suggest that ASTL may really be a meprin, with plasma membrane isoforms. Underlying Aim 1 is that SAS1B isoforms encoded by different ASTL splice variants traffic to different sites including the plasma membrane. Moreover, single copy ASTL gene encodes both secreted and plasma membrane isoforms. This application will study the protein trafficking of the different human ASTL splice variants in Aim 1.

C.1.1 Splicing of Human ASTL.

Six astl splice variants have been reported previously in mice. Similarly, from normal human ovaries as well as from human tumors we have identified and sequenced six ASTL splice variants using RACE PCR (see FIG. 13). FIG. 13 shows the exon-intron arrangements in splice variants A through F [SV-A-SV-F], and indicates the number of amino acids, predicted masses, and isoelectric points of the deduced SAS1B proteins (all of which preserve the canonical HExxHxxxxXH zinc metalloproteinase catalytic motif). The 9 exon human ASTL gene undergoes very interesting alternative splicing resulting in two distinct groups of predicted proteins. In the first group, SV-A & B have classical signal peptides encoded by exon 1. The signal peptides in SV-A & B possess a canonical scission sequence and are predicted to be cleaved during protein processing. In contrast, in the second group SV-C-F do not use exon 1 (indicated by black blocks for E1) but rather variants C-F use a different start site that is located in intron 1, which, in effect, extends exon 2 (see FIG. 13, SV-C-F). The N-terminus sequence that is added from intron 1 gives splice variants C-F an entirely different 28 amino acid N-terminus than that which is found in SV-A & B. The new N-terminal sequence in SV-C-F is hydrophobic overall and aa 10-24 are predicted to encode an α-helical transmembrane domain. This new N-terminal hydrophobic domain lacks a scission sequence typical of signal peptides and is predicted to persist in each SAS1B protein encoded by SVC-F and to function as a single pass transmembrane domain in these 4 isoforms.

In sum, from the deduced sequences of the different ASTL splice variants in man, two groups of SAS1B proteins can be distinguished, each group predicted to engage in different routes of trafficking and to differ in cellular locations. Group 1 SAS1B isoforms, comprised of splice variants A and B, are expressed with classical signal sequences that direct their entry into the endoplasmic reticulum and Golgi apparatus and are predicted to represent secreted SAS1B isoforms. Group II, comprised of splice variants C-F, contains isoforms with a single pass transmembrane domain at their N termini and are predicted to insert into membranes without being cleaved and to represent integral membrane isoforms found at tumor cell surfaces. For example, FIG. 14 shows the sequence of SV-C, its domain structure, homology model, and relationship to the plasma membrane. Splice variants C-F orient with 9 N-terminal amino acids located intracellularly; a hydrophobic domain of amino acids 10-24 spanning the membrane; a proximal extracellular domain of unordered amino acids from aa 25-65; and the catalytic domain as well as the unique unordered C-terminal domain exposed extracellularly. Proteins encoded by SV-C-F would thus be classified as Type II membrane proteins. Aim 1 defines which ASTL splice variants encode proteins are secreted or inserted into the plasma membrane while Aim 3 defines which splice variants and isoforms are expressed in various OUCs and in what proportions. As will be shown below, mAbs to the N-terminus of SAS1B (that map between aa 29 & 148 in SV-C-D and 29-145 in SV-A & B) show striking cytotoxicity as duocarmycin conjugates on SAS1B+ tumor cells.

PCR Assays for ASTL and its Splice Variants.

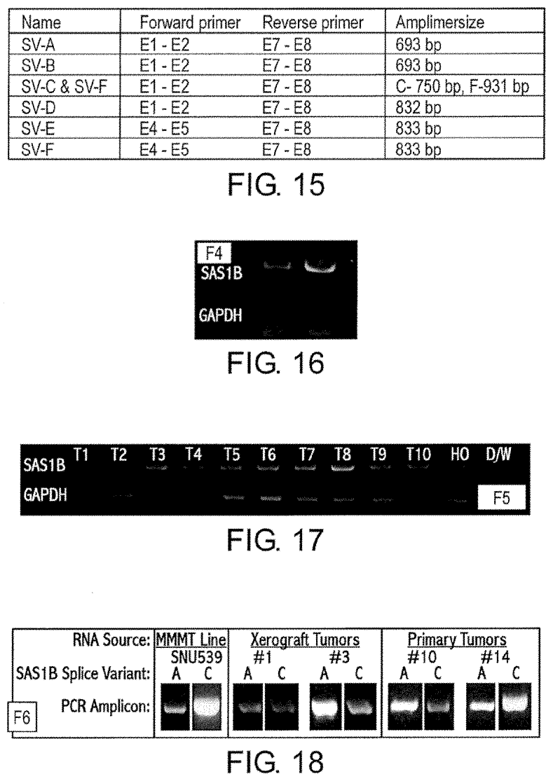

Primers to ASTL sequences that are common in all 6 human splice variants have been synthesized and a method to amplify all 6 isoforms at the same time has been developed (pan-ASTL PCR). Using a combination of unique forward and reverse primers, methods have also been developed that amplify each of the 6 isoforms separately. FIG. 15 shows the exon-intron splice junctions spanned by the primers and the amplimer characteristics. The PCR primer sets designed for this study have been validated by cloning and sequencing amplimers obtained from more than 30 tumors. Each primer set amplifies only ASTL sequences and not those of any of the other 134 metalloproteases in the human genome. FIG. 16 shows PCR amplification of a 310 bp pan-ASTL amplimer using ASTL specific c-terminus primers from two samples of normal human ovarian cDNA. Normal ovary serves as one of the positive controls in these assays. The PCR assay for the 310 bp amplimer was used to study ASTL transcripts in 15 cases of advanced, disseminated serous ovarian cancer, of which 10 samples (harvested from the mesenteries) are shown in FIG. 17 (T1-10). ASTL transcripts were seen in all ten as well as in the positive control human ovary (HO). The reverse transcriptase alone control (D/W) showed no band. Overall, transcripts encoding SAS1B were found in 13/15 stage III and IV serous tumor specimens examined (86% incidence rate). The amplimers were cloned and sequenced, and each represented authentic ASTL with 99% fidelity. Each person's ASTL sequence differed only in an occasional SNP. FIG. 18 shows the amplification of SV-A and SV-C, in the uterine cancer MMMT line SNU-539, primary tumor specimens, and xenografts. The results demonstrate that tumors not only express different ASTL splice variants but that the relative abundance of ASTL splice variants differs from tumor specimen to specimen. ASTL PCR studies carried out previously with a pilot cohort of uterine cancers were classed as Malignant Mixed Mullerian Tumors (MMMT) and endometrioid tumors [76].

qPCR Assay for ASTL in Tumors.

A specific and sensitive quantitative RT-PCR assay for ASTL/SAS1B mRNA expression has been developed. Standard qRT-PCR techniques which employ oligo-dT or random oligo priming of cDNA synthesis by reverse transcriptase (RT) lacked the sensitivity and dynamic range for quantification in a range of samples with limited amounts of starting material. The technique that was developed uses gene-specific priming after high temperature denaturation of total RNA template and annealing of a gene-specific reverse primer prior to transcription by a thermostable reverse transcriptase.

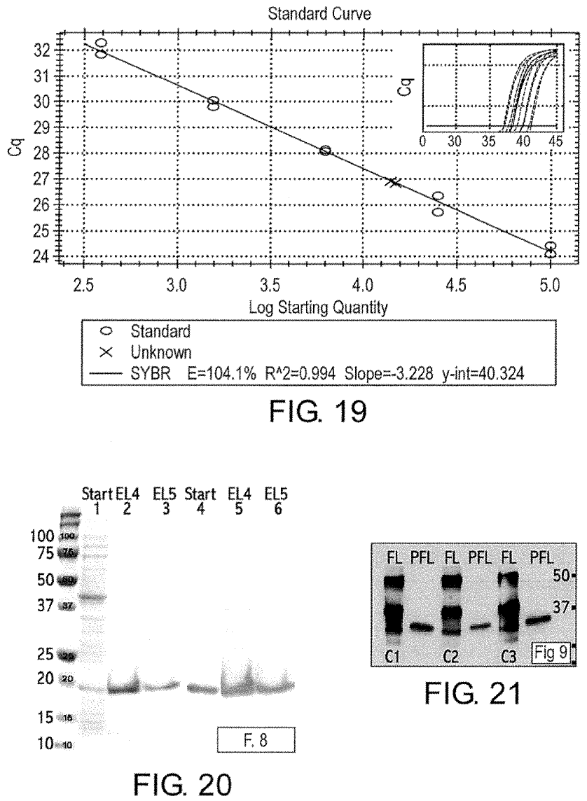

Two different primer sets that amplify different regions of the ASTL cDNA have been characterized. One set consists of a forward primer lying in exon 2 and a reverse primer in exon 3; the second set uses a forward primer in exon 5 and a reverse primer in exon 6. Both primer sets span an intron, eliminating the possibility of erroneous amplification of genomic DNA contaminants. In addition, both of these exon junctions are common to all known splice variants of ASTL mRNA, allowing the quantification of total ASTL expression. To facilitate absolute quantification of mRNA expression, a synthetic RNA standard was produced by in vitro transcription of an ASTL cDNA clone. This standard is then diluted in a series of different concentrations containing yeast RNA as a carrier, in order to maintain a constant amount of total RNA in each qRT-PCR reaction. qRT-PCR is performed on this dilution series in parallel to sample RNAs. Real-time detection (FIG. 19, inset) of PCR amplification is accomplished by inclusion of Sybr-green in the PCR reaction. FIG. 19 shows a qRT-PCR of the exon 5-6 product for a standard curve produced by a 4-fold dilution series of synthetic ASTL RNA (∘) run in conjunction with an RNA sample from the SNU-539 uterine cancer cell line (X). The standard curve starts at 100,000 copies of ASTL RNA and descends to 1562 copies in the beginning RT reactions. FIG. 19 demonstrates that the assay is quantifiable over a 44 or 256-fold range. The PCR methods described here was used in Aim 3 to define the different SAS1B isoforms expressed in tumors and permit personalized diagnostics that determined which tumors transcribe ASTL mRNAs encoding surface isoforms of SAS1B.

Recombinant Expression Vectors and Purified Human SAS1B Proteins.

Mammalian expression constructs for SV-A, SV-C and truncated versions including only the C-term region of the extracellular domain have been made with, and without, V5 and 6×-His tags. Human SAS1B proteins have been expressed in HEK293T cells and milligram quantities of SAS1B protein have been purified by metal affinity and gel filtration chromatography. FIG. 20, for example, shows the 19 kDa SAS1B C-term extracellular domain purified from HEK-293 cells. Lane 1 shows the whole cell protein lysate (starting material), lanes 2 & 3 show protein staining of fractions eluted from the nickel affinity column, lane 4 is the anti-his tag western blot of the recombinant protein in the starting lysate, and lanes 5 & 6 show the anti-his western blots of the purified proteins. The high purity of recSAS1B in the fractions (2 & 3) may be appreciated. Bacterial expression constructs for SV-A as well as truncated SV-A lacking both signal peptide and propeptide domains have also been created with 6×-His tags, and SAS1B protein has been purified from bacteria (see FIG. 21). Full length (FL) 49 kDa recombinant SAS1B undergoes partial autoproteolysis and the 49 kDa form runs with several breakdown products, while the truncated recombinant protein (PFL) runs at 36 kDa. These various recombinant mammalian and bacterial human SAS1B proteins have been used as immunogens in mice and as targets for screening murine and rabbit mAbs and for production of polyclonal antibodies in rabbits. In addition, SV-A and SV-C constructs with SAS1B linked to a mCherry reporter have also been created. These fusion proteins have been used to study SAS1B subcellular localization and in Western blotting studies.

Anti-Human SAS1B mAbs.

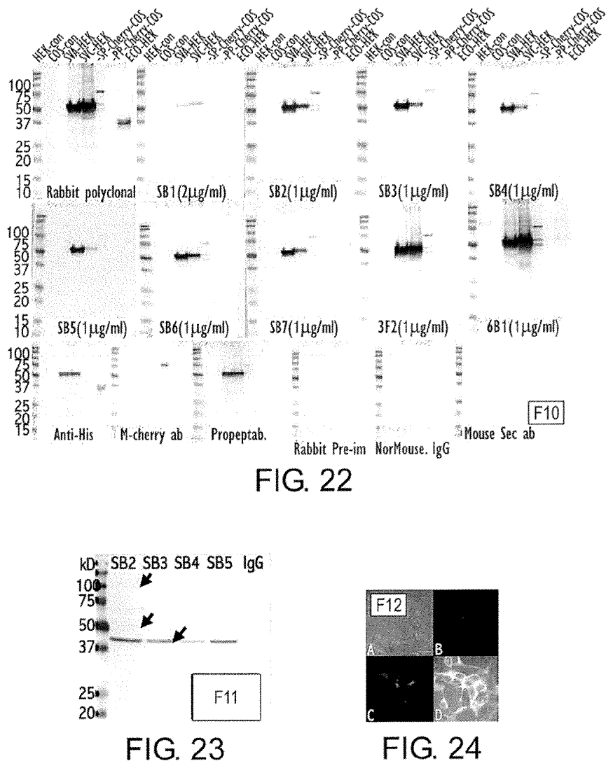

Six hybridoma fusions in mice have been conducted to date with full length, truncated, and C-term (aa 160-431) extracellular domain SAS1B immunogens. Rabbit mAbs have also been produced by cloning from antigen-specific B lymphocytes and more than 30 stable rabbit mAbs have been cloned. More than 60 stable murine hybridomas secreting anti-SAS1B mAbs have been identified and mAbs purified by Protein A affinity chromatography after scale up in gas permeabilized bags. FIG. 22 shows Western blots loaded with recombinant SAS1B SV-A (aa 1-431), SV-C (aa 1-436), mCherry-SAS1B fusion protein, and C-terminus extracellular domain (aa 160-431). A rabbit polyclonal antibody generated to rec SV-A reacted (FIG. 22, top left) with the SV-A, SV-C, mCherry fusion protein and C-term extracellular domain. mAbs SB1-7, 3F2 and 6B1 all recognized SV-A and SV-C proteins but did not react with the C-term extracellular domain, placing the epitopes for this series of mAbs between aa 23-160. Control preimmune, isotype matched monoclonals and secondary antibody alone blots were all negative while positive control anti-his, anti-M-Cherry and anti-SAS1B pro-peptide reagents were all positive. This data demonstrates a capability to isolate mAbs to recombinant SAS1B and to map their epitopes using domain deletion constructs.

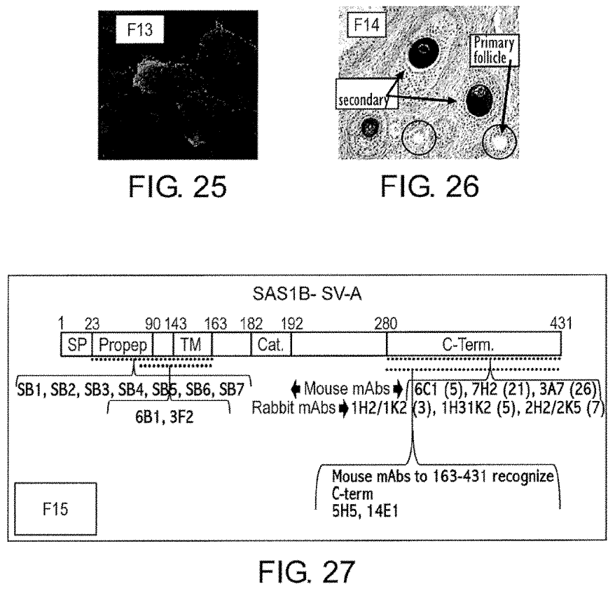

Bioassays to select therapeutic and diagnostic mAbs have focused on two strategies: 1) identification of mAbs reactive with recombinant SAS1B, endogenous SAS1B, and with SAS1B accessible on cell surfaces; and 2) mAbs that recognize epitopes preserved in formalin fixed paraffin embedded sections that work as immuno-cytochemical probes. FIG. 23 demonstrates mAbs SB2-5 reacting with endogenous 44 kDa SAS1B extracted from uterine cancer cells. FIG. 24 is a confocal image of one mAb, SB4, recognizing native SAS1B in fixed cells transfected with V-5 tagged SAS1B, and showing where the mAb to SAS1B (FIG. 24, bottom left) co-F 13 localized with an anti-V5 antibody (FIG. 24, top right). Non-transfected cells showed no SAS1B signals. These transfected cells provide one effective screening tool to identify mAbs reactive with native epitopes on SAS1B. Another screening assay is to study SAS1B+ tumor cell lines by immunofluorescence microscopy and FACS. mAbs that recognize SAS1B at the surface of live cells and gate specific cell populations by FACS have been isolated. For example, in FIG. 25, the SB2 monoclonal is seen in confocal immunofluorescence microscopy to bind SAS1B on live cells. Regions at the cell surface where filopodia are prominent demonstrate concentrations of SAS1B immunofluorescence. Efforts to identify mAbs that localize SAS1B in fixed embedded sections have also yielded reagents of utility for histopathology. FIG. 26 shows staining for SAS1B protein on a paraffin section of macaque ovary using IgG1K mAb 6B1 (1:16,000 dilution). SAS1B was localized only in the large growing eggs in secondary follicles (black arrows) while SAS1B was not present in the smaller eggs within primary follicles (circles). Thus, macaque SAS1B was observed to be restricted to the large growing oocytes and no expression of SAS1B protein was observed in quiescent eggs that comprise the reserve of resting oocytes, matching the results reported in humans. Thus the non-human primate ovary can be used as a positive control tissue for studying the histopathology of tumor specimens with these reagents.

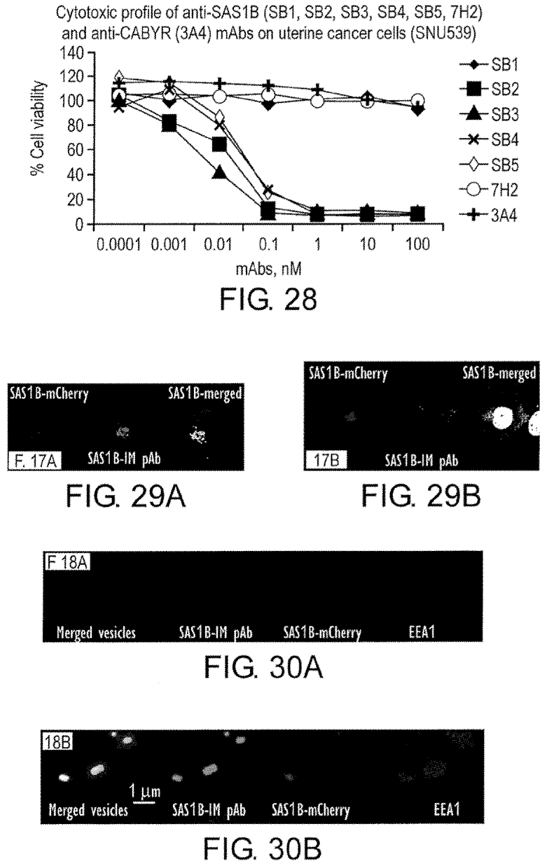

These results confirm the availability of mAbs to screen fixed tissues embedded in paraffin (Aim 3). FIG. 27 summarizes the regions of SAS1B to which some of the leading mouse and rabbit mAbs have been mapped. MAbs 3F2 and 6B1 (above) that have proven useful for immunohistochemical localization are reactive with N-terminal epitopes (FIG. 24). Significantly, several mAbs in the SB series, directed to the N-terminus, have proven particularly effective as antibody-duocarmycin conjugates in killing uterine tumor cells in vitro as shown in FIG. 28. Cell killing effects of anti-SAS1B mAbs SB 2, 3, 4 & 5 on SAS1B+ human tumor cells were achieved at sub-nanomolar concentrations while isotype matched mAb 7H2, to the sperm antigen CABYR, and the SB1 mAb reacting with mouse SAS1B, showed no cytotoxic effects. Although mAbs to the SAS1B C-terminus have been tested for cytotoxic effects as antibody-duocarmycin conjugates, to date all mAbs that possess cytotoxic properties as ADCs bind to the N-terminal 29-145/8 amino acids encompassing the proximal membrane and the propeptide domains (FIG. 14) in SVC-F.

C.2 Research Strategy, Milestones, Impact on Future Direction.

This proposal identifies ASTL splice variants that encode SAS1B isoforms accessible at the surfaces of cancer cells, select mAbs to epitopes that induce cell internalization and cell killing, employ quantitative PCR assays and mAbs to detect and measure ASTL splice variants and SAS1B proteins in tumor samples, and determine the frequency of SAS1B transcription and translation in OUC of various stages. Aim 1 uses a variety of cell biological methods to identify which ASTL splice variants encode proteins that traffic to the cell membrane. Aim 2 produces murine mAbs to SAS1B surface epitopes (ectodomains), study the accessibility of the target epitopes at tumor cell surfaces, and evaluate the mAbs as histopathological probes and their capacity to deliver cytotoxic payloads. mAbs that possess properties to kill cancer cells in vitro have their CDR regions sequenced. Aim 3 determines the frequency of SAS1B transcription and translation in OUC of various stages and develops knowledge on SAS1B splice variants in tumors.

Impact.

If incidences of SAS1B expression seen in pilot cohorts of OUC remain at the high levels observed in preliminary results, SAS1B will emerge as an exciting new OUC target. Identification of mAbs that bind SAS1B ectodomains and have tumoricidal activity as ADCs creates a library of antibodies that can subsequently be tested in mouse xenograft models, humanized, and/or used as targeting elements for CART vector construction. SAS1B emerges as a “platform target” around which a range of therapeutic approaches are built. Complementarity determining regions (CDRs) of the effective murine mAbs identified are humanized by grafting onto a human immunoglobulin scaffold to create non-immunogenic recombinant humanized antibodies or engineered as T-cell receptors or chimeric antigen receptors.

Aim 1: Determine which Alternative Splice Variants of ASTL Encode SAS1B Protein Isoforms Accessible at the Tumor Cell Surface.

Rationale.

As shown in preliminary results, six alternative splice variants of ASTL have been identified, cloned and sequenced in human ovaries and in human tumors. These variants are predicted to encode both secreted SAS1B proteins and SAS1B isoforms that orient as Type II integral membrane proteins. Qualitative PCR studies confirmed that tumors express multiple ASTL splice variants and that variants detected in tumors include those that are predicted to translate into both secreted and integral membrane isoforms. In order to correctly phenotype patient's tumors to direct targeted therapy and to select mAbs that bind to relevant epitopes at the cell surface it is crucial to determine which of the SAS1B protein isoforms localize to the plasma membrane and to understand the topology of different SAS1B isoforms in relationship to the cell membrane. We have developed domain specific mAbs (FIG. 27). These antibodies are used with the panel of tests described below to study the surface accessibility of SASIB isoforms.

Approach.

To identify SAS1B protein isoforms accessible at the cell surface, cDNA encoding splice variants A-F were cloned into reporter constructs to express a chimeric protein with a mCherry tag at the C-terminus. These tagged fusion proteins were expressed in Cos-7 cells, which do not express endogenous SAS1B, and a multipronged approach was used to test for localization of the expressed SAS1B proteins at the plasma membrane; namely: 1) FACS analysis, 2) internalization/endocytosis assays, 3) assessment of the SAS1B internalization pathway; 4) super resolution and confocal microscopy, and 5) surface biotinylation-detection of surface biotinylated proteins. Exposure of C-term tag epitopes at the cell surface as detected by FACS, microscopy, and verified by cell internalization and surface biotinylation served formal proofs of the surface accessibility of a specific isoform. Additionally, the mAbs to different domains of SAS1B shown in preliminary results were tested in Cos-7 cells transfected with different human splice variants to determine which antibodies bind at the cell surface, thus verifying both the capacity of a monoclonal antibody to bind to specific SAS1B isoforms at the cell surface and confirming which domains are accessible.

Feasibility.

FIG. 29A shows the internal Golgi pool of SAS1B SV-A (middle) in Cos7 cells co-localized with SAS1B-C-term mCherry (left) signal after cell permeabilization with TX-110. Right hand panels (FIG. 29B) show the surface pool of SAS1B SV-C with no permeabilization. FIGS. 30A and 30B demonstrate application of this reporter strategy in studies of SAS1B internalization. No staining with mCherry antibody, SAS1B antibody, and endosomal marker EEA1 is noted in Cos-7 cells transfected with the empty vector (FIG. 30A). However, in cells transfected with the SV-C isoform of SAS1B (FIG. 30B), the SAS1B antibody complex is endocytosed and merges with EEA1 vesicles after 15 minutes. These transfection experiments with epitope tagged SAS1B splice variants in Cos-7 cells demonstrate feasibility of the proposed approach and show that SAS1B isoforms A & C traffic to different locales.

Specific Methods: Constructs & Transfection.

cDNAs encoding full-length human SAS1B mCherry-tagged at the C-terminus were prepared for all 6 isoforms. Cos-7 cells were transfected by electroporation.

1) FACS & Analysis.

Surface expression of the different SAS1B isoforms were investigated using live cell flow cytometry. In brief, transfected cells were allowed to recover for 2 hours followed by blocking and antibody staining in chilled azide based medium for 2 hours. After washes with chilled media, cells were incubated with goat anti-rabbit and anti-mouse RPE for 1 hour in blocking media, washed and fixed with 4% PFA, and read on the flow cytometer. Data was analyzed by the FlowJo program.

2) Endocytosis/Internalization Assessed by Microscopy.

Four predicted SAS1B Type II transmembrane proteins have a small (9 aa) intracellular N-terminal region, a transmembrane domain close to the N-terminus and a large extracellular C-terminal region (FIG. 14). As described earlier, SAS1B isoforms tagged at the C-terminus exposing the tag extracellularly and making it easily accessible for anti-cherry antibody to bind and stimulate protein internalization by endocytosis. For such studies, Cos-7 cells were grown on coverslips, starved in serum free media for 2 hours and then incubated on ice for 20 min to immobilize the plasma membrane. Cells were then incubated on ice for 1 hour with anti-cherry antibody (1:1000), washed three times with cold DPBS, and then either stopped at time 0 by fixing cells with 4% PFA or transferred to fresh prewarmed complete Roswell Park Memorial Institute medium (RPMI) at 37° C. to allow antibody internalization to proceed for 5, 15, 30 or 60 min. The samples were subsequently fixed in 4% PFA, washed twice with DPBS and permeabilized with 0.1% Triton X-100 in PBS for 15 min. Next, cells were washed with DPBS and exposed to GαRb Alexa Fluor488 secondary antibodies diluted 1:500 for 60 min at 37° C. After final washes with DPBS, cells were mounted in slow-fade with DAPI (Molecular Probes, OR, USA) and imaged. Additionally, colocalization studies were performed with confocal microscopy at each of the mentioned time points to test if internalized SAS1B isoform colocalized with EEA1 and LAMP2, markers of early and late endosomes respectively.

3) Assessment of the SAS1B Internalization Pathway.

Endocytosis is a process by which a cell actively internalizes membrane bound proteins or extracellular cargo into the cell. Of the many kinds of endocytic pathway, clathrin mediated endocytosis seems to be predominant route by which the cell internalizes membrane proteins. We tested if any of the SAS1B isoforms are internalized by clathrin mediated endocytosis. Cos-7 cells expressing individually mCherry-tagged SAS1B isoforms were grown on coverslips, fixed and permeabilized as previously describe and incubated with anti-cherry antibodies and antibodies to clathrin or AP-2 (proteins involved in clathrin mediated endocytosis). Cells were washed and subsequently treated with secondary antibodies, washed again, and mounted in slowfade as described earlier. Images were captured using confocal microscopy. Further, live cell imaging by TIRF microscopy, which acquires images at a high axial resolution of 100 nm, were used to observe the packaging of SAS1B isoforms into clathrin coated pits as they got endocytosed at the plasma membrane. As an additional test, cells were depleted of clathrin using lentiviral mediated knockdown and internalization/endocytosis assays were performed as described above. Membrane bound isoforms were retained on the plasma membrane in cells depleted of clathrin.

4) Super Resolution Imaging of Surface Localization and Endocytic Processes of SAS1B Isoforms.

Those SAS1B isoforms which localized at the plasma membrane and which significantly colocalized with clathrin/AP2 were tested further using super-resolution approaches to better resolve the localization of the SAS1B isoforms at the plasma membrane. SAS1B variants were cloned with photo-switchable proteins such as Dendra2 (using pDendra2-N vector from Clontech) which is a monomeric fluorescent protein derived from octocoral (Dendronephthya sp). The Dendra2 sequence has been optimized for maturation and a bright fluorescence both before and after photoswitching. Its codon usage is optimized for high expression in mammalian cells. Transfected cells as described above were seeded on coverslips at a density of 10,000 cells and allowed to grown for 12-24 hours. Conventional IIF protocol were employed on formaldehyde fixed cells and membrane associated SAS1B protein and its isoforms were observed using the stochastic optical reconstruction microscopy (STORM) achieving thousands of high resolution images in the range of 30 nm. In parallel, classical antibody mediated endocytosis assay with co-localization with markers of the endoreticular system were employed and internalized SAS1B vesicles were visualized with high resolution confocal microscopy (Pires et al., 2015).

5) Live Cell Imaging.

Two color live cell imaging/spinning disc microscopy were used to trace the endocytic path of anti-SAS1B mAbs. Specifically, the heavy chains of were labelled with Alexa 488 fluorescent dyes using the Site Click™ labelling technology from Thermo fisher. Tumor cell lines known to express specific SAS1B isoforms were transfected with mCherry EEA1/LAMP2. These cells were fed with dye conjugated domain specific mAbs and the fate of the mAbs were imaged live as they traffic to the late endosomes.

6) Biochemical Proof of Surface Localization.

For surface biotinylation, Pierce™ Cell Surface Protein isolation kit were used. Briefly, Cos7 cells individually expressing SAS1B-cherry tagged isoforms (SVA-F) were treated with cell-impermeable, cleavable biotinylation reagent (Sulfo-NHS-SS-Biotin) to label exposed primary amines of proteins on the surface of intact adherent cells. Treated cells were harvested, lysed and the labeled surface proteins affinity-purified using NeutrAvidin Agarose Resin. The biotinylated protein enriched on the resin were eluted and resolved using SDS-PAGE, transferred to nitrocellulose membrane and probed with anti-cherry antibody for immunostaining.

Endpoints and Impact.

Preliminary results showed that tumors and tumor cell lines expressed multiple SAS1B splice variants encoding different protein isoforms. The results from Aim 1 identified which SAS1B splice variants encoded SAS1B proteins that are accessible at the cell surface. Moreover, using domain-specific mAbs, those SAS1B regions that were accessible at the cell surface were defined and the overall orientation of domains with respect to the cell membrane was determined, allowing models of the orientation of each SAS1B isoform with respect to the cell membrane. The knowledge gained in Aim 1 shaped the selection of mAbs to be used as targeting and diagnostic reagents.

Aim 2: Develop mAbs Reactive with Surface Exposed SAS1B Epitopes, Test their Biological Properties and Map their Epitopes.

Rationale.

Specific SAS1B domains and their epitopes are associated with the cell membrane and mAb binding with select epitopes can mediate anti-tumor immune mechanisms. By identifying mAbs to SAS1B ectodomains, determining which antibodies fix complement and have cytotoxic effects, and mapping their epitopes, SAS1B epitopes mediating anti-tumor activities were identified. As shown, a range of mAbs to different SAS1B domains has already been achieved. Additional regents were developed and tested in the assays below to develop a range of candidate targeting agents to different ectodomains.

Approach.

To identify mAbs that bind readily to SAS1B epitopes accessible at the tumor cell surface, each hybridoma supernatant was evaluated in a series of 5 primary screening assays: 1) ELISA selection on purified recombinant SAS1B targets; 2) immunofluorescence microscopy staining of SAS1B+ tumor cells; 3) two color FACS analysis using Cos or HEK 293 cells transfected with a specific SAS1B isoform bearing a reporter tag; 4) Western blotting to extracts from SAS1B+ cells including native (non-reduced) and reduced protein preparations; and 5) immunohistochemistry on formaldehyde fixed paraffin embedded SAS1B+ cells. Hybridomas secreting mAbs meeting any one of the above criteria was cloned and supernatants retested in the primary screens for retention of immunological activity. Stable cloned hybridomas were grown in gas permeable bags in defined media. Antibodies were purified by HPLC on Protein G columns. Purified mAbs were then tested in secondary screens (below).

Specific Methods.

Five ovarian and 2 uterine tumor cell lines (SKOV3, P76, P66, OVT53, R182, 539, 308) have been identified that express human SAS1B and display SAS1B on their cell membranes. In addition, Cos-7 and HEK293 cells have been transfected with various SAS1B isoform constructs containing 6-his and V5 tags. SAS1B

Antigen Preparations.

To optimize the identification of mAbs to surface exposed epitopes on human SAS1B, immunization of mice occurred with several SAS1B antigens: 1) recombinant human SAS1B expressed in bacteria; 2) various purified recombinant SAS1B isoforms expressed in HEK293 cells; and 3) native SAS1B isolated from tumor cells by immunoprecipitation. More than 30 mg of the SB3, 4 & 5 mAbs have been harvested and shown to immunoprecipitate “native” SAS1B expressed in HEK 293 cells. These mAbs were used to immunoprecipitate native SAS1B from scaled up cultures of 539 cells for the third immunogen.

Immunization.

Purified SAS1B antigens were injected into mice following an immunization protocol that utilizes intra-muscular and sub-cutaneous primary and secondary immunizations, and intra-splenic immunization via a percutaneous route, 5 days prior to splenocyte harvest for hybridoma fusion.

Primary Screens.

ELISA screens consist of standard 96 well plate coating assays using purified recombinant SAS1B produced in mammalian and bacterial expression systems. Immunofluorescence confocal microscopy was used to demonstrate that a mAb binds to the cell membrane of SAS1B+ cells that are transfected with constructs expressing various SAS1B isoforms containing a V5 reporter tag. The mAb must co-localize in SAS1B+ cells, which were verified using an antibody to the V5 tag, while neither the mAb nor the anti-V5 tag mAb binded to un-transfected cells. Confocal microscopy demonstrated that a mAb binds to surface membranes of live SAS1B+ tumor cells or SAS1B+ cells transfected with various SAS1B constructs, but does not bind to the cell surfaces of SAS1B− tumor cells or cells transfected with vector alone. FACS proofs consisted of showing a mAb binds to live (and/or fixed) SAS1B+ tumor cells or to SAS1B+ transfected cells, and does not bind to SAS1B− cells. This demonstration was followed by the proof that the mAb can be used to sort SAS1B+ cells from SAS1B-cells in an equal mixture of the 2, with enrichment of SAS1B protein demonstrated by western blot, ELISA, or MS/MS. A FACS Calibur equipped with a 96 well plate automated acquisition unit was employed. Each monoclonal was tested in triplicate wells. Methods demonstrating Western analyses and immunohistochemistry were shown. Those hybridomas secreting mAbs that tested positive were cloned, stabilized, scaled-up in gas permeable bags, and antibodies purified by HPLC on Protein A columns. The purified antibodies were tested in secondary screens.

Secondary Screens.

Immunochemical proofs of mAb specificity consisted of demonstrating that the mAb immunoprecipitates endogenous SAS1B from SAS1B+ cells. Proteins were extracted from SAS1B+ cells with non-ionic detergents, the mAb was used to immunoprecipitate SAS1B, with the recovery of SAS1B in the immune complex shown by western blots demonstrating SAS1B stained with peptide or polyclonal antibody, and by recovery of SAS1B peptides in MS/MS analysis of tryptic fragments of the immune complexes. Another experiment tested if a mAb will immunoprecipitate vectorially labeled SAS1B extracted from SAS1B+ cells. Cells expressing surface SAS1B were biotinylated with vectorial reagents to label SAS1B; surface proteins were extracted from cells with a mild non-ionic detergent and immunoprecipitated with anti-SAS1B mAbs; and biotinylated SAS1B was recovered, and analyzed by western blotting using avidin-peroxidase. Cell surface biotinylation was performed on live tumor cells using the cell impermeable, water soluble sulfo-NHS-biotin (Nhydroxysuccinimide biotin) which labels external lysines. Human SAS1B (SV-A: 431 aa) contains 10 well distributed lysines (positions: 53, 93, 111, 175, 220, 277, 352, 353, 394, 426).

Complement Dependent Cytotoxicity (CDC).

The Roche Excelligence impedence electrode system was used to assess the effect of mAbs on cell vitality in the presence of active and inactive complement. Markers of apoptosis were employed to verify cytotoxic effects.

Conjugations.

Anti-SAS1B mAbs was coupled, via linker arms, with drug payloads to create active directly conjugated ADCs. ADCs were created with drug payloads including the microtubule disruptors monomethyl auristatin E & F (MMAE & MMAF) and the DNA intercalating drug duocarmycin. Cathepsin sensitive cleavable linker arms were conjugated to Duocarmycin DM (Moradec).

Cell Lines.

The aim used ovarian SKOV3 and R182 cells, uterine MMMT 539 and 308 tumor cell lines and HEK293 and Cos-7 cells that express ASTL transcripts and display SAS1B protein on the cell surface.

Cell Internalization and the Endocytic Pathway.

To study internalization and co-localization of monoclonal antibody-SAS1B complexes with endocytic markers, cells were grown to 50% confluence, starved 2 hours in serum-free media, and incubated with primary polyclonal and mAbs to SAS1B after having been cooled to 1-4° C. on ice, in order to restrict lateral mobility of proteins in the plane of the membrane. Antibody concentrations over the micromolar to nanomolar range were employed to optimize signals. Cells were then warmed and at various time points (5, 15, 30, and 60 minutes) after reaching 37° C., they were fixed and the location of antibodies binding to SAS1B was monitored. SAS1B positive vesicles within the cytoplasm were taken as evidence of internalization. These vesicles were small and localized at the cell periphery in the early time points and coalesced into larger vesicles and be found deeper within the cytoplasm with longer time periods. mAbs to SAS1B, directly labeled with fluorescent reporters, were used to monitor the internalization process with spinning disc confocal microscopy. Next, at various time points after warming, SAS1B was co-localized with clathrin, caveolin, EEA1 and LAMP1, markers of the early and late arms of the endocytic pathway. Confocal microscopy was used to verify co-localization.

ADCs.

MAbs identified was custom conjugated with various drug payloads and used to study effects on tumor cell growth in vitro. Conjugates were tested for retention of biological activity against native SAS1B in ELISA and by live cell indirect immunofluorescence surface staining. For initial dose ranging studies, mAbs was tested over the micromolar to picomolar range. Negative control arms included drug conjugates of isotype matched mAbs to irrelevant antigens (CABYR & GFP). SAS1B-cells were also employed as a negative control. The positive control was treatment of cells with TX100 detergent or etoposide to induce cell lysis at the beginning of the treatment phase.

Total ATP as a Measure of Cytotoxicity.

The amount of ATP is proportional to the number of cells in culture. Human cancer cells were seeded 1500/well in triplicate in 96 well plates in and allowed to recover overnight. The next day antibody drug conjugates (100, 10, 1, 0.1, 0.01, 0.001, 0.0001 and 0.0 nM final concentration) were added to the cells in 10 ul media, mixed and incubated for ˜93 h at 37° C. On Day 4, the presence of viable cells per well were determined by measuring the total cellular ATP using the luciferin luciferase system CytoTox Glo from Promega. Relative luminescence units (RLU) were determined in a Biotek cytation3 luminometer. Viability of control live cells (100%) were determined from average of 9 wells. Controls included isotype matched mAbs, the drug alone, and baseline medium alone. Staurosporin, a general protein kinase inhibitor known to induce apoptosis, was used in 1% DMSO as a positive control in the assay for cytotoxicity. An ATP standard curve from 0 to 1000 nM was used to calibrate the assay. The sulforhodamine B (SRB) assay serves as a backup method.

Monoclonal antibody epitopes were mapped by demonstrating binding or an absence of binding to various recombinant deletion constructs using Western blot and dot blot analyses and by array-based oligo-peptide scanning on a library of oligo-peptide sequences from overlapping and non-overlapping segments of SAS1B. Monoclonal antibody binding was quantitated by ELISA. A homology model for SAS1B was used in conjunction with the estimated lengths of peptide antigens binding in the complementarity determining regions to deduce and synthesize potential discontinuous epitopes by combining non-adjacent peptide sequences from different parts of SAS1B and enforcing conformational rigidity onto this combined peptide (by using CLIPS scaffolds). In this way discontinuous epitopes were mapped with very high reliability and precision. This method is fast and relatively inexpensive, and specifically suited to profile continuous and discontinuous epitopes for large numbers of candidate antibodies. Affinity determinations (Kd) of mAbs reacting with SAS1B employed surface plasmon resonance with a Biacore instrument.

Endpoints and Impact.

Our demonstrated ability to make mAbs to recombinant SAS1B, to map mAb binding domains (FIG. 27), and to assess the cytotoxicity of mAb-duorcarmycin conjugates (FIG. 28) indicate that this aim is feasible. Aim 2 projected to yield a panel of high affinity anti-SAS1B mAbs reactive with both continuous and discontinuous epitopes on SAS1B ectodomains. Antibodies were identified that internalize into the endosomal-lysosomal system after cell surface binding and demonstrate cytotoxicity in SAS1B positive ovarian and uterine tumor cell lines. Some of these mAbs showed immunological activity in fixing complement and inducing complement dependent cytotoxicity, while others were suitable as immunoreagents on fixed sections. The complementarity determining regions of the mAbs were sequenced. By mapping the epitopes recognized by these mAbs, the key SAS1B domains accessible on the surface of tumor cells were identified and related to tumoricidal functions. Thus a map of the relevant SAS1B epitopes accessible at the tumor cell surface emerged.

Aim 3: Determine the Incidence of ASTL Expression, Including Alternative Splice Variants and SAS1B Protein Isoforms, in a Variety of Ovarian and Uterine Cancers.

Rationale.

ASTL is transcribed and translated at high frequency in OUCs, with several splice variants and SAS1B protein isoforms being expressed, and SAS1B expression related to histologic cell type, grade, and potentially stage and pattern of metastatic spread. At least 870 OUC specimens were obtained and tested for ASTL mRNA and SAS1B protein levels. The most common histological subtypes of ovarian (serous, endometrioid, and clear cell) and uterine (endometriod, MMMT, serous papillary, and clear cell carcinomas) were examined as well as representative samples of the much rarer germ cell tumors and sex cord tumors. Understanding splicing and SAS1B protein isoforms is essential information to have in designing a mAb that was used for diagnostic and therapeutic purposes as it provided a comprehensive understanding of which protein domains are present in the target tumor cells, provided for selection of mAbs that had the broadest reactivity to tumor cells and thus the broadest potential therapeutic and diagnostic utility.

Approach.

The study (target N=870) retrospectively (N=750) studied 350 archival OUC fixed anatomical pathology specimens and 400 frozen tumor specimens, and studied 120 newly consented OUC specimens. Whole sections of tumor blocks and tumor tissue microarrays were utilized to assemble a variety of tumor types for side by side comparison using standard immunohistochemical and in-situ hybridization protocols. Serial sections in triplicate for each treatment allowed reproducibility assessment. Staining and staging was read by two independent pathologists blinded to each other's data and results were analyzed for congruence (κ). Laser capture micro-dissections were used to circumscribe tumorous and non-tumor regions in select specimens. Archival frozen tissues were studied by quantitative RT-PCR, qRT-PCR, and Western blotting. Gene specific primers, PCR, and PCR RACE were used to amplify alternative splice variants. RNAseq were undertaken on six specimens each year (N=30).

Specific Methods.

Prospective specimens were sampled and dissected, when possible, so that both tumor RNA and fixed, embedded tissue was available for each specimen. For specimens where tumor boundaries are grossly visible, dissection into “tumor” and adjacent “non-tumor” tissues was made. Laser capture microdissection retrieved regions of embedded specimens containing tumorous tissue and adjacent regions of putatively normal tissue to allow comparisons of SAS1B RNA profiling and results from histochemical localization and in-situ hybridization. For histology, tissues were fixed in NBF and embedded in paraffin. Frozen archived tumor specimens were processed for RNA extraction using the Quiagen RNeasy Protect Mini Kit using RNeasy spin columns. Specimens were stored in RNAlater RNA Stabilization Reagent when stabilization of RNA in tissue samples is required. Standardization and quality control conditions for PCR included consistent use of 1 ug RNA for cDNA synthesis and use of control GAPDH primers to verify RNA quality and load equalization. All samples followed an identical PCR program with constant cycle number and reagents, and the PCR amplimers from 1 out of every 5 samples were authenticated by cloning and sequencing and comparison to the reference sequence. Human ovarian cDNA served as the positive control for PCR reactions. All mRNA and cDNA samples were saved for subsequent qRT-PCR studies to define CT values.

Gene-specific primers and PCR RACE were used to amplify alternative splice variants which were cloned and sequenced to identify human SAS1B isoforms. Anti-sense in-situ hybridization probes were obtained from Advanced Cell Diagnostics and labeled with colorimetric and FISH reporters. RNA-sequencing, a powerful emerging technology for quantitative transcriptome profiling, was used to capture SAS1B splice variants by a largely hypothesis-independent approach. RNA was isolated, depleted of ribosomal RNA, adapters ligated to the 3′ end of the mRNA, and primed using this adapter sequence for synthesis of cDNAs by thermostable reverse transcriptase. cDNA amplimers were paired-end sequenced and expressed sequences assembled to produce an inventory of mRNA isoforms. Expression levels of the transcript variants were also quantified. To confirm the accuracy of RNA-seq analysis of alternative splicing, a subset of tumors and ovarian cDNA were amplified using semi-quantitative RT-PCR.

Additional PCR reactions using primers designed to flank identified splice variants were used to confirm results from RNA sequencing and to generate amplicons for subcloning and sequencing. Proficiency with PCR and immunohistochemical methods have been demonstrated, including detailed studies of splice variants and in situ hybridization methods. Standardization of immunohistochemical staining methods included the use of preimmune sera as the negative control for polyclonal immune sera, isotype matched negative controls for mAbs, and the inclusion of positive control human or monkey ovary tissue in every tumor microarray experiment. Positive controls for in situ methods included use of GAPDH probes and negative control scrambled probes. A “+, ++, and +++” grading system for hybridization and histochemical results was used. This was referenced on the intensity of human and macaque oocyte staining in ovary sections which were scored as +++. In large histologic sections, patterns of heterogeneity of expression were also assessed. For serum antibodies, identical experimental and control dilutions were made. Pathological assessments were provided and specimens classified following the WHO Nomenclature, assessing type, grade, and the clinical and surgical records correlated to assess tumor stage, the latter to follow the FIGO nomenclature.

Analysis and Interpretation.

Data obtained included intensity of staining scores following in situ-hybridization and immunohistochemistry on whole sections and microarrays, detection of individual splice variants, protein expression of individual protein isoforms, and tumor stage, grade and histologic cell type. Patient records were studied to determine the correlations between variables such as ASTL expression, expression of different isoforms, SAS1B protein expression, cell type localization, tumor classification, and clinical outcomes such as responsiveness to treatment, recurrence and survival. Data was stored in tabular form as a matrix. Of particular interest was overall incidences of SAS1B in OUC subtypes, and whether particular subsets of these cancers were particularly prone to express SAS1B. Expression of SAS1B in metastatic tumors isolated from different organs was of particular interest. Frequent expression of SAS1B in metastatic specimens from disseminated sites suggested targeting SAS1B to treat advanced disease.

Endpoints and Impact.

This aim delivered detailed information on the incidence of SAS1B expression in OUC cancers, identified specific SAS1B isoforms found in OUC, defined cancer subtypes in which expression occurs, and correlated SAS1B expression with metastasis and response to therapy. Since SAS1B expression is understood to be qualitatively different between normal tissues and the tumor, the development of diagnostic methods to detect the presence of SAS1B in OUC provided for a differential diagnosis, and governed future decisions to treat a patient with a SAS1B targeted therapy. PCR assays developed in this aim for SAS1B splice variants were important diagnostics to selectively identify patients expressing cell surface SAS1B isoforms permitting stratification of patients who may benefit from targeted SAS1B immunotherapy. Determination of the incidence of SAS1B expression in OUC of various types, and the identification of precisely which splice variants are present in which tumors, helped validate SAS1B as a diagnostic and therapeutic target for different OUC types, grades and perhaps stages. Data regarding SAS1B expression in different types of OUC supported FDA applications to gain clearance for experimental trials of a SAS1B IT in select indications.

Overall Deliverables.

As an oncology target, the SAS1B surface metalloprotease offers a possible pathbreaking tumor-selective mechanism of drug action that targets only tumor cells and an expendable population of growing oocytes while sparing the ovarian reserve and preserving fertility. The forms of SAS1B present on tumor cell surfaces are defined, leading to precision diagnostic assays. Monoclonal antibodies to SAS1B ectodomains were created that bind to tumor cell surfaces, internalize, and deliver cytotoxic payloads. These can become immunotherapeutics. Importantly, the overall incidence of expression of the ASTL gene and its splice variants in different ovarian and uterine cancers were gained so that a firm understanding can be reached regarding which tumors could be treated and how many women may be helped by a SAS1B-targeted therapy.

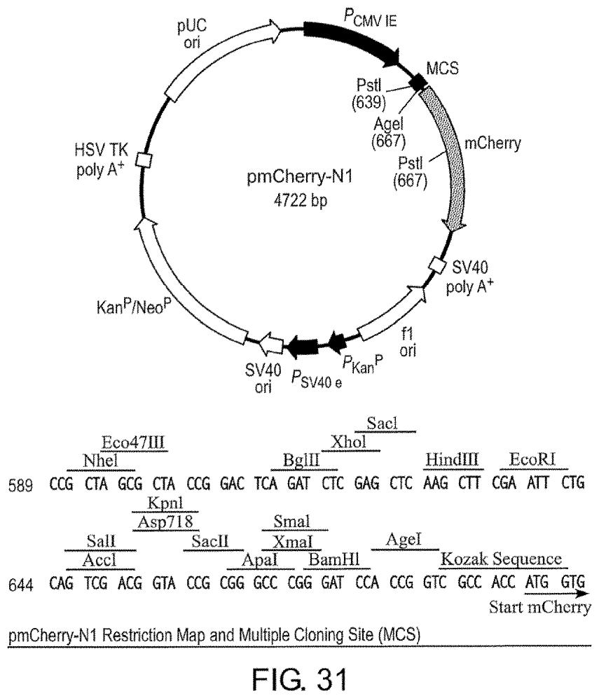

FIGS. 31-47D disclose data and other information supporting aspects of the embodiments already described with respect to FIGS. 12A-30B.

FIG. 31 shows primers with restriction sites designed to amplify (a) the full length gene (1-431); (b) mutant SAS1B without signal peptide domain (1-23 deleted); and (c) mutant SAS1B without signal and pro-peptide domains (1-90 deleted). PCR products were cloned into m-Cherry N1 vector to express a fusion protein at the N-term of mCherry (C-term of SAS1B has mCherry). SAS1B-Cherry engineered DNA was transfected in COS cells to express fluorescent tagged protein.

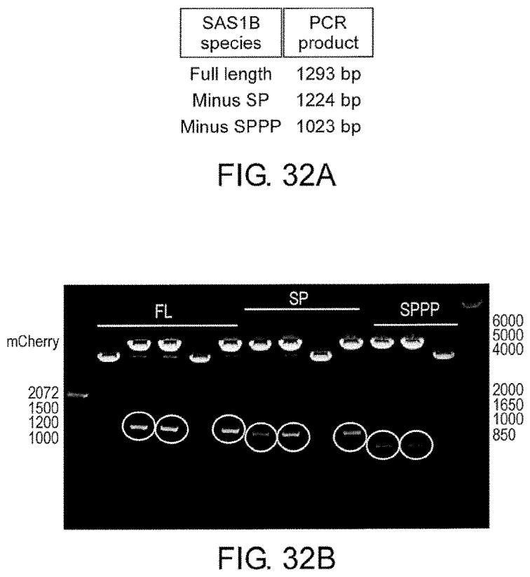

FIGS. 32A and 32B show full length & truncated mutant SAS1B species, wherein FL correlates to full length SAS1B, SP correlates to minus signal peptide domain of SAS1B, and SPPP correlates to minus signal and propeptide domains of SAS1B.

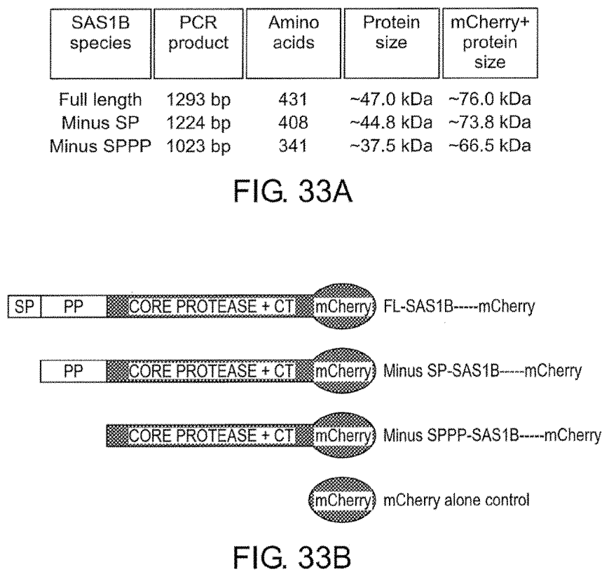

FIGS. 33A and 33B show FL and mutant SAS1B constructs.

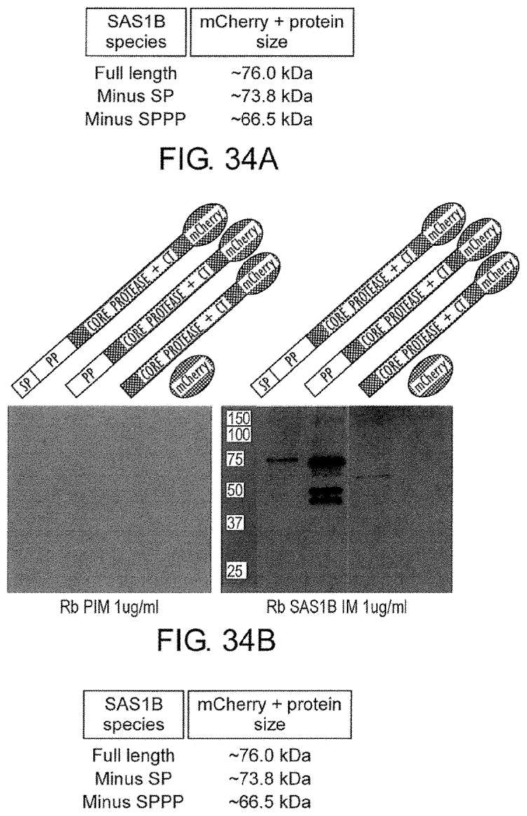

FIGS. 34A and 34B show protein expression analyses wherein (a) 1 million transfected cells were harvested in Laemmli buffer containing BME; (b) 20 ul was loaded in wells of a SDS-PAGE followed by transfer and Western blotting; (c) membranes blocked with 5% NFDM-PBS; (d) primary antibody: Rb PIM/IM @ 1 ug/ml conc, O/N, 4 C; (e) washes followed by secondary antibody Goat anti-Rb HRP 1:20,000, 1 hr RT; and (f) washes followed by ECL detection. Rb=Rabbit polyclonal antibody; PIM: pre immune, control antibody; IM: immune, test antibody.

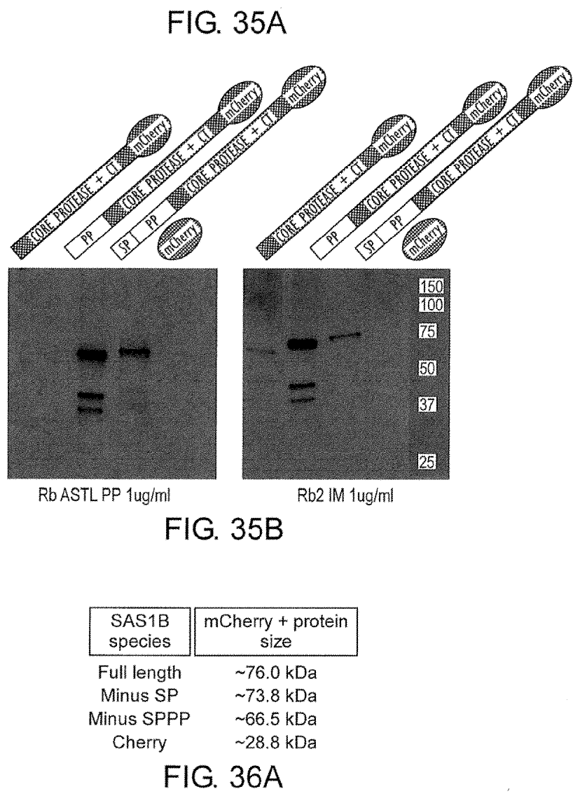

FIGS. 35A and 35B show protein expression analyses wherein (a) 1 million transfected cells were harvested in Laemmli buffer containing BME; (b) 20 ul was loaded in wells of a SDS-PAGE followed by transfer and Western blotting; (c) membranes blocked with 5% NFDM-PBS; (d) primary antibody: Rb ASTL pro-peptide, Abcam and Rb2 IM @ 1 ug/ml conc, O/N, 4 C; (e) washes followed by Secondary antibody Goat anti-Rb HRP 1:20,000, 1 hr RT; and (f) washes followed by ECL detection. Rb=Rabbit polyclonal antibody; PIM: pre immune, control antibody; IM: immune, test antibody.

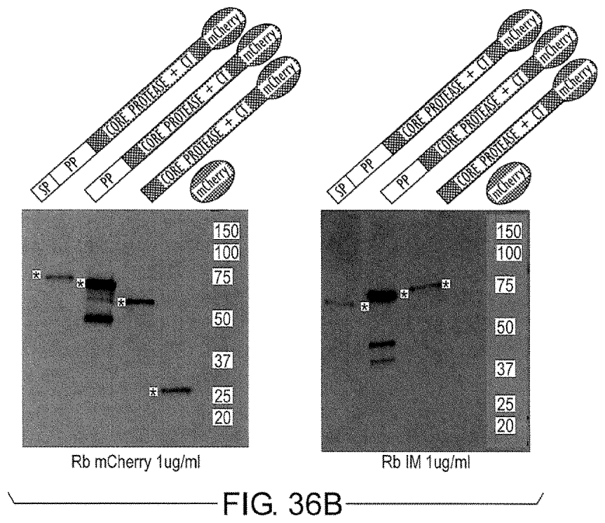

FIGS. 36A and 36B show protein expression analyses wherein (a) 1 million transfected cells were harvested in Laemmli buffer containing BME; (b) 20 ul was loaded in wells of a SDS-PAGE followed by transfer and Western blotting; (c) membranes blocked with 5% NFDM-PBS; (d) primary antibody: Rb mCherry, BioVision and Rb IM @ 1 ug/ml conc, O/N, 4 C; (e) washes followed by Secondary antibody Goat anti-Rb HRP 1:20,000, 1 hr RT; (f) washes followed by ECL detection. Rb=Rabbit polyclonal antibody; PIM: pre immune, control antibody; IM: immune, test antibody.

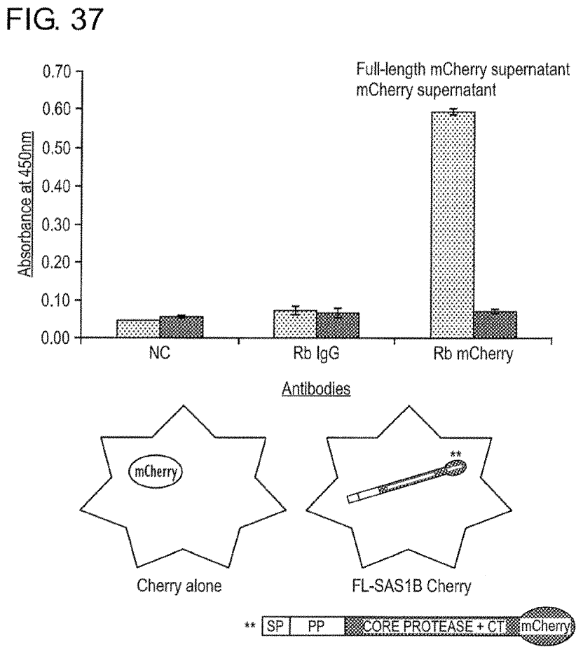

FIG. 37 shows free SAS1B in spent medium wherein (a) supernatants from phenol red-free DMEM were collected post 48 hr of transfection (24+24); (b) 7 ml of supernatant was concentrated to 300 ul and then proteins were precipitated in TCA-Acetone; (c) 100 ug/100 ul protein coated in ELISA well plates with Carbonate-bicarbonate buffer O/N, 4 C; (d) wells were blocked with 10% NGS/5% NFDM-PBS, 1 hr RT; (e) primary antibody: Rb mCherry, BioVision @ 0.2 ug/100 ul conc in duplicates, 37 C, 2 hrs; (f) washes followed by 100 ul of secondary antibody, Goat anti-Rb HRP 1:5000, 1 hr RT; (g) washes followed by 100 ul of TMB substrate, 37 C 5 mins and then stopped in 100 ul of 1N HCl, A450 nm; and (h) values plotted on Excel spreadsheet.

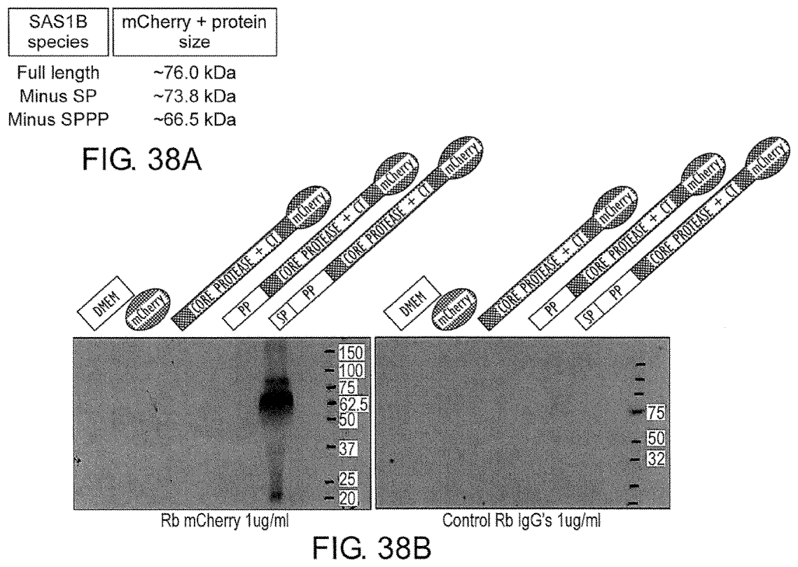

FIGS. 38A and 38B show spent media Western blot wherein (a) supernatants from phenol red-free DMEM were collected post 48 hr of transfection (24+24); (b) 7 ml of supernatant was concentrated to 300 ul and then proteins were precipitated in TCA-Acetone; (c) 100 ug protein was loaded in a SDS-PAGE, transferred to CN membrane and blocked with 10% NGS/5% NFDM-PBS, 1 hr RT; (d) primary antibody: Rb mCherry, BioVision @0.2 ug/100 ul conc in duplicates, 4 C, O/N; (e) washes followed by Secondary antibody Goat anti-Rb HRP 1:20,000, 1 hr RT; and (f) washes and then detected by ECL. From FIGS. 38A and 38B, (a) the antibody detects several forms of the fusion protein; (b) a dominant ˜66 kDa form which corresponds to the Minus SPPP mutant which is also the active form of ASTL/SAS1B; (c) a ˜76 kDa which is the full length form of ASTL/SAS1B; and (d) lower forms are seen around ˜35 kDa and ˜20 kDa.

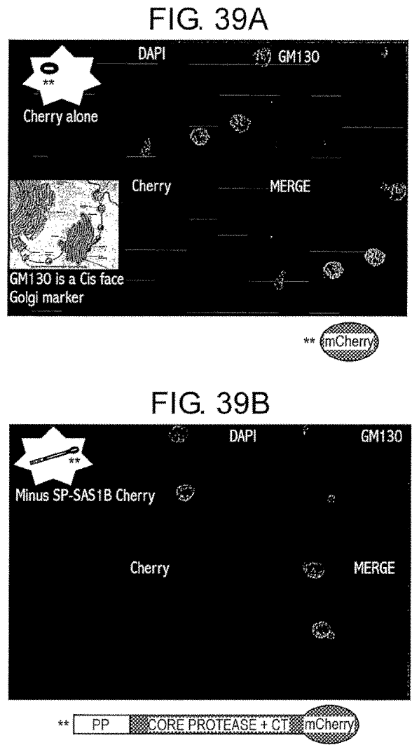

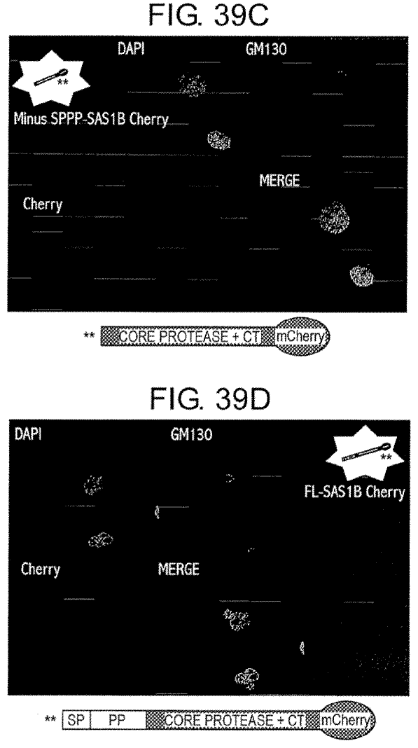

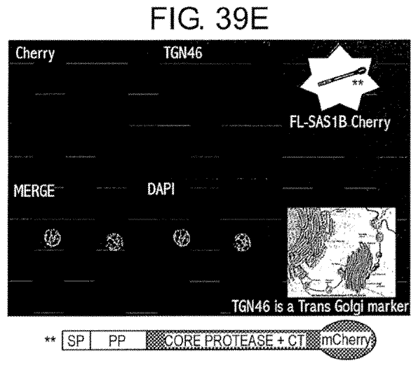

FIGS. 39A-39E shows SAS1B localization methods wherein (a) 1 million COS cells were counted/transfection; (b) cells re-suspended in Amaxa transfection buffer containing 1 ug engineered DNA; (c) transfection by electroporation; (d) seed transfected cells in DMEM and plate in dishes; and (e) next day, split and seed 20,000 cells on slips for IIF; and (f) cells were fixed with 4% PFA-PBS, 15 mins RT followed by imaging or co-localization as indicated.

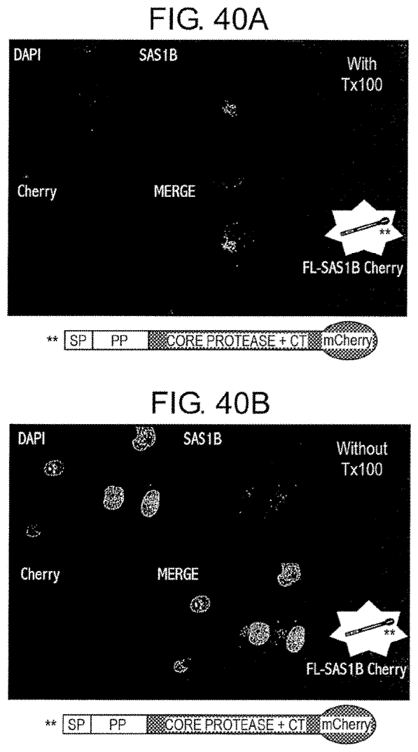

FIGS. 40A and 40B show surface and cytoplasmic forms wherein (a) post transfection with FL-SAS1B cherry constructs COS cells were treated by (i) blocking in BSA containing TritonX 100. Fixed with 4% PFA-PBS and stained with Rb IM Ab (4 ug/ml). All buffers were made with PBS-Tx100. TritonX100 allows large molecules such as antibodies to enter the inside of the cell. And (ii) blocking in BSA. Fixed with 4% PFA-PBS and stained with Rb IM Ab (4 ug/ml). All buffers were made with PBS alone (NO Tx100). The detection antibody was Goat-anti rabbit Alexa 488 (green) 1:500 in PBS. All Abs incubated for 1 hr at RT.

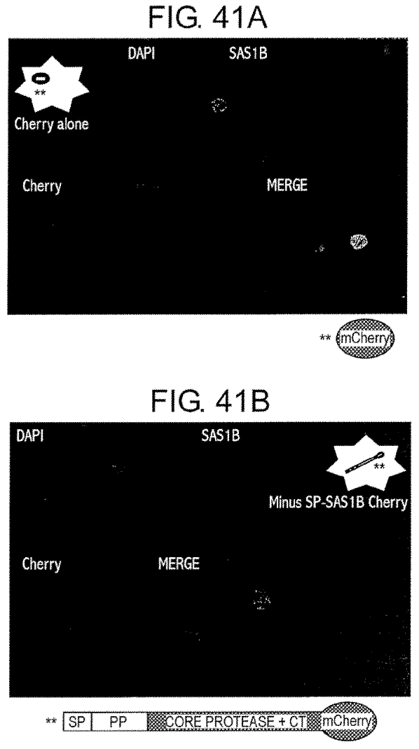

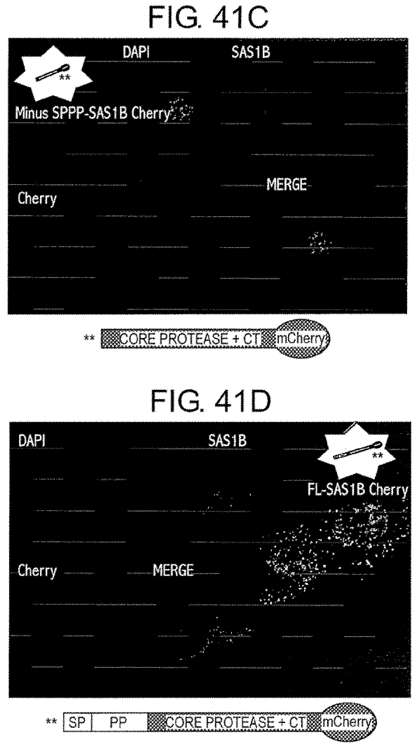

FIGS. 41A-41D show internalization wherein (a) Cos cells with the below constructs were transfected as described previously; (b) mCherry antibody at a concentration of 1 ug/ml was incubated with the cells for 1 hr at 37 C; (c) cells were washed and fixed with 4% PFA in a PBS-Tx100 solution; and (d) detected with Goat anti-Rabbit Alexa 488 (green) 1:500 1 hr, RT.

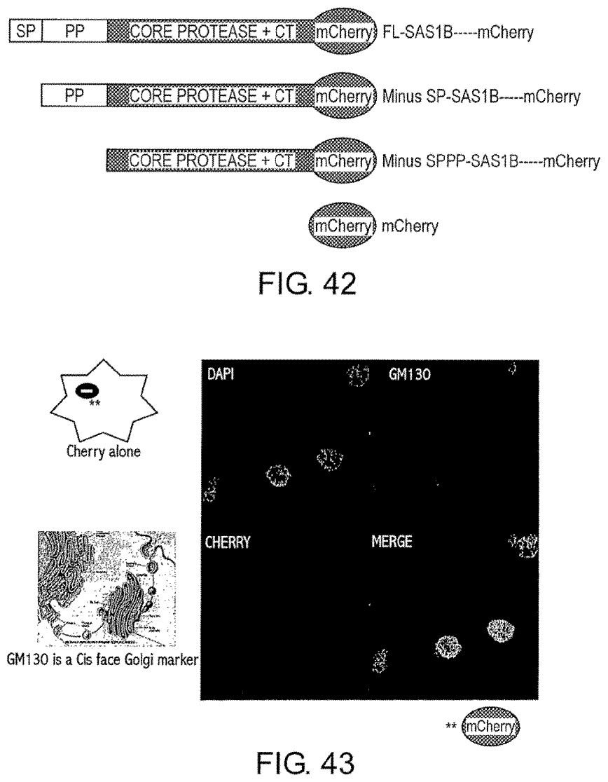

FIG. 42 shows FL and mutant SAS1B constructs.

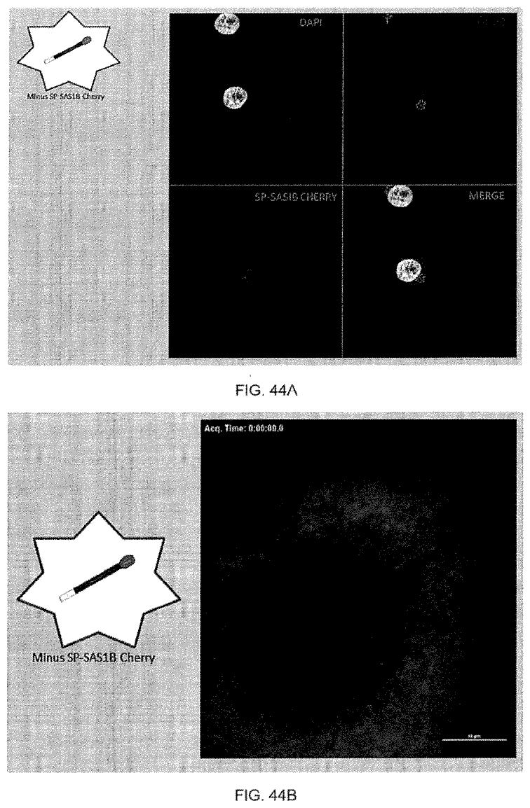

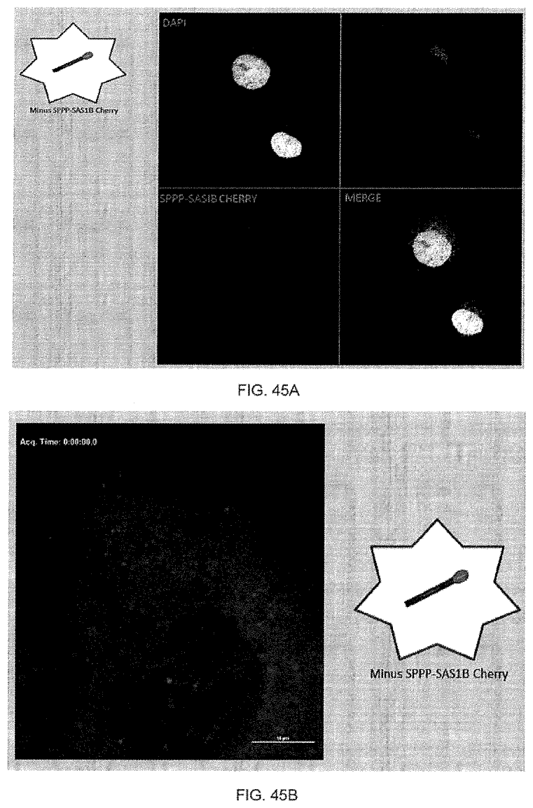

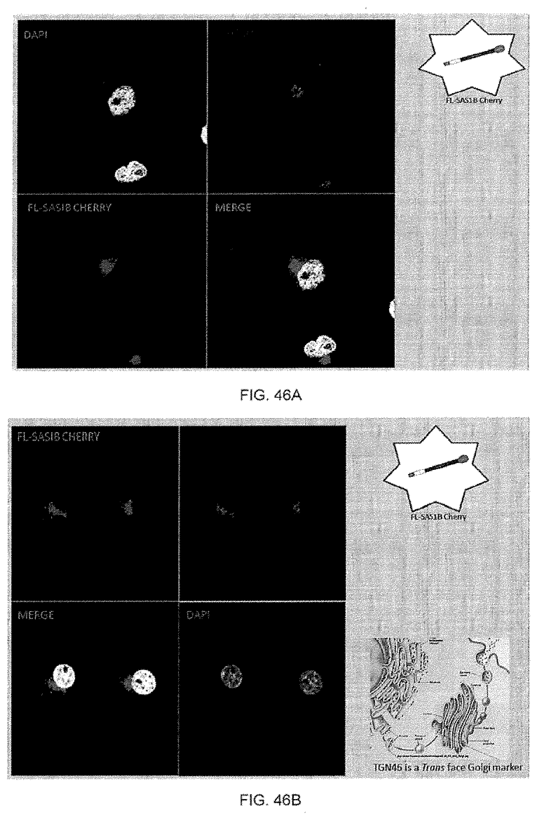

FIG. 43 shows mCherry alone expression on transfection, where the protein tends to be arrested in nucleus and not in the cytoplasm.

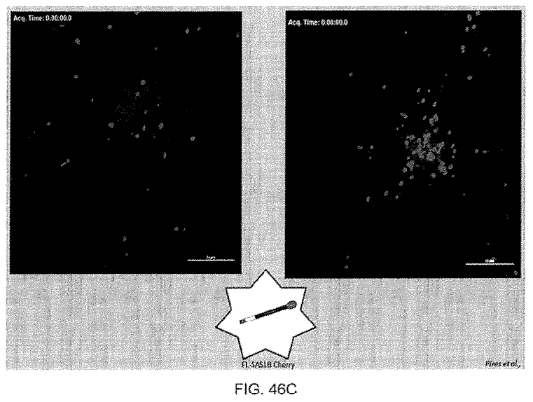

FIG. 44A shows that SP-SAS1B appears to be cytosolic; does not enter the ER-GC route; and there is no co-localization with GC marker. FIG. 44B shows real time profiling of SP-SAS1B Cos cells.

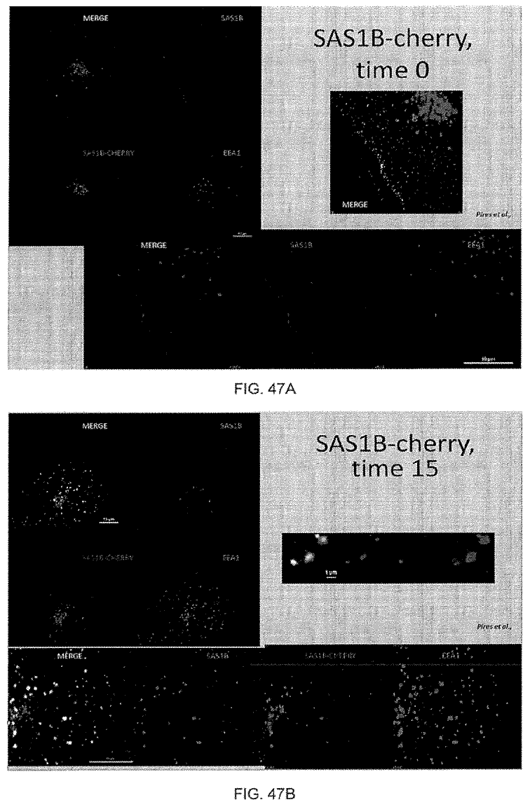

FIG. 45A shows that SPPP-SAS1B appears to be cytosolic; does not enter the ER-GC route; and there is no co-localization with GC marker. FIG. 45B shows real time profiling of SPPP-SAS1B Cos cells.

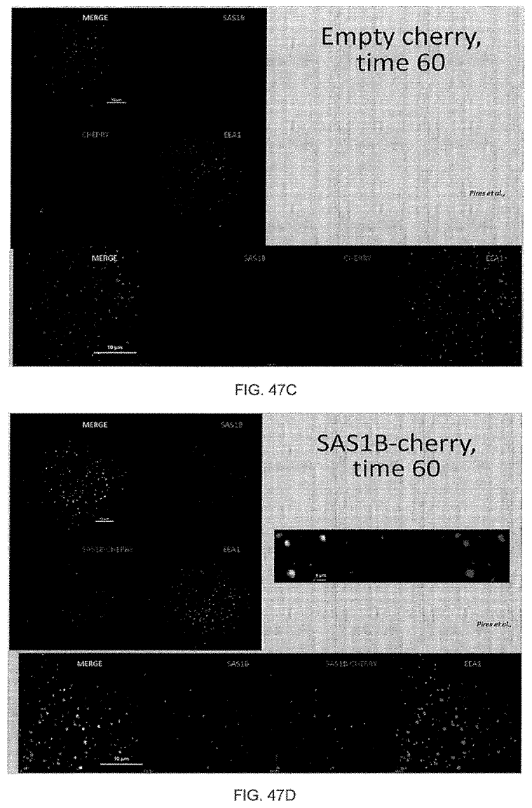

FIGS. 46A and 46B show co-localization of SAS1B protein with two independent Golgi markers indicating protein gets trafficked into this compartment prior to the membrane. FIG. 46C shows real time profiling of FL-SAS1B Cos cells.

With respect to FIGS. 43-46C, at steady state, over-expressed SAS1B-cherry protein appears to be present in the reticular system as well in the perinuclear region (Golgi Complex+endosomes). Furthermore, without the signal peptide as well as the pro-peptide domains, SAS1B protein seems to be arrested in the cytosol and does not enter the perinuclear pool and thereby does not get trafficked to the membrane.

FIGS. 47A-47D show endocytosis wherein (a) transfected Cos7 cells (empty and SAS1B-cherry); (b) anti-mCherry antibody 1:1000 dilution on ice for 1 hour; (c) warm slides at 15 mins and 60 mins at 37 C; (d) fix with PFA; (e) co-localization with mouse EEA1 for 1 hr 1:300; (f) anti-Rabbit green and Anti-mouse Far-red (blue); and (g) image on confocal 100×, 1125×1125 resolution, 70 nM/pixel, 0.5 micron optical slice.

With respect to FIGS. 47A-47D, (a) FL-SAS1B protein gets trafficked to cell membrane; (b) in the presence of antibody, SAS1B gets internalized by 15 mins and coalesces with the early endosomal pathway (EEA1); and (c) endocytosed SAS1B vesicles continue to be present in vesicular structures and likely enters late endosomal pathway by 60 mins.

Those skilled in the art will appreciate that, given the disclosure herein, modification may be made to the invention without departing from the spirit of the inventive concept. It is not intended that the scope of the disclosure be limited to the specific and preferred embodiments illustrated and described. All documents referenced herein are hereby incorporated by reference, with the understanding that where there is any discrepancy between this specification and the incorporated document, this specification controls.

1. (canceled)

2. The method of claim 22, wherein the antibody comprises at least one of:
(a) a VH CDR1 of SEQ ID NO:1, a VH CDR2 of SEQ ID NO:2, a VH CDR3 of SEQ ID NO:3, a VL CDR1 of SEQ ID NO:4, a VL CDR2 of GAS, a VL CDR3 of SEQ ID NO:5;
(b) a VH CDR1 of SEQ ID NO:1, a VH CDR2 of SEQ ID NO:6, a VH CDR3 of SEQ ID NO:7, a VL CDR1 of SEQ ID NO:8, a VL CDR2 of KVS, a VL CDR3 of SEQ ID NO:9; or
(c) a VH CDR1 of SEQ ID NO:1, a VH CDR2 of SEQ ID NO:2, a VH CDR3 of SEQ ID NO:10, a VL CDR1 of SEQ ID NO:11, a VL CDR2 of KVS, a VL CDR3 of SEQ ID NO:9.

3-6. (canceled)

7. The method of claim 22, wherein the antibody or antigen-binding portion thereof is a monoclonal antibody, a chimeric antibody, a humanized antibody, a synthetic antibody, a single chain antibody, a diabody, or a CDR-grafted antibody.

8. The method of claim 22, wherein the antibody or antigen-binding portion thereof comprises a VL amino acid sequence of SEQ ID NOs:13, 15, 17, or 18.

9. The method of claim 22, wherein said antibody or antigen-binding portion thereof comprises the VH amino acid sequence of SEQ ID NOs:12, 14, or 16.

10-11. (canceled)

12. The method of claim 22, wherein the antibody or antigen-binding portion thereof specifically binds human SASiB with an affinity (K_{d}) of at least about 10⁻⁶ M.

13. The method of claim 22, wherein said antibody or antigen-binding portion thereof binds to cancer cells.

14-20. (canceled)

21. The method of claim 22, wherein the antibody is a chimeric antibody comprising VL and VH domains obtained from a mouse antibody, wherein said VL and VH domains include sequences capable of binding to human SAS1B, and the VL and VH domains are fused to human CL and CH domains, respectively.

22. A method of treating a hyperproliferative disorder comprising:
administering a composition to a mammal in need thereof, the composition comprising:
(a) an antibody or antigen-binding portion thereof and a pharmaceutically acceptable carrier; or
(b) the antibody or antigen-binding portion thereof, wherein the antibody or antigen-binding portion thereof is conjugated to a therapeutic agent, and a pharmaceutically acceptable carrier,
wherein the antibody or antigen-binding portion thereof comprises:
a VH CDR1 of SEQ ID NO:1; a VH CDR2 of SEQ ID NO:2 or 6; a VH CDR3 of SEQ ID NO:3, 7, or 10; a VL CDR1 of SEQ ID NO:4, 8, or 11; a VL CDR2 of GAS or KVS; and a VL CDR3 of SEQ ID NO:5 or 9 or 95% identity thereto.

23. (canceled)

24. A method of detecting SAS1B-positive cells in a test sample comprising:
(a) contacting one or more antibodies with the test sample under conditions that allow SAS1B-positive cell/antibody complexes to form; and
(b) detecting SAS1B positive cell/antibody complexes;
wherein
the antibody comprise:
a VH CDR1 of SEQ ID NO:1; a VH CDR2 of SEQ ID NO:2 or 6; a VH CDR3 of SEQ ID NO:3, 7, or 10; a VL CDR1 of SEQ ID NO:4, 8, or 11; a VL CDR2 of GAS or KVS; and a VL CDR3 of SEQ ID NO:5 or 9 or 95% identity thereto, and
the detection of SAS1B positive cell/antibody complexes is an indication that SAS1B cells are present in the test sample.

25. The method of claim 24, wherein the sample is lymph node or tissue aspirate, serum, whole blood, cellular suspension, lymphocytes, whole blood, plasma, circulating tumor cells, tumor cells or tissue, ascites fluid, urine, or fluid effusion.

26-32. (canceled)

33. The method of claim 24, wherein the antibody comprises at least one of:
(a) a VH CDR1 of SEQ ID NO:1, a VH CDR2 of SEQ ID NO:2, a VH CDR3 of SEQ ID NO:3, a VL CDR1 of SEQ ID NO:4, a VL CDR2 of GAS, a VL CDR3 of SEQ ID NO:5;
(b) a VH CDR1 of SEQ ID NO:1, a VH CDR2 of SEQ ID NO:6, a VH CDR3 of SEQ ID NO:7, a VL CDR1 of SEQ ID NO:8, a VL CDR2 of KVS, a VL CDR3 of SEQ ID NO:9; or
(c) a VH CDR1 of SEQ ID NO:1, a VH CDR2 of SEQ ID NO:2, a VH CDR3 of SEQ ID NO:10, a VL CDR1 of SEQ ID NO:11, a VL CDR2 of KVS, a VL CDR3 of SEQ ID NO:9.

34. The method of claim 24, wherein the antibody is a monoclonal antibody, a chimeric antibody, a humanized antibody, a synthetic antibody, a single chain antibody, a diabody, or a CDR-grafted antibody.

35. The method of claim 24, wherein the antibody comprises a VL amino acid sequence of SEQ ID NOs:13, 15, 17, or 18.

36. The method of claim 24, wherein said antibody comprises the VH amino acid sequence of SEQ ID NOs:12, 14, or 16.

37. The method of claim 24, wherein the antibody specifically binds human SAS1B with an affinity (K_{d}) of at least about 10⁻⁶ M.

38. The method of claim 24, wherein said antibody binds to cancer cells.

39. The method of claim 24, wherein the antibody is a chimeric antibody comprising VL and VH domains obtained from a mouse antibody, wherein said VL and VH domains include sequences capable of binding to human SAS1B, and the VL and VH domains are fused to human CL and CH domains, respectively.


SEQUENCE LISTING (36 sequences)

SEQ ID NO: 1 - protein, 8 aa, Mus musculus
GYTFTDYN

SEQ ID NO: 2 - protein, 8 aa, Mus musculus
INPNNGGT

SEQ ID NO: 3 - protein, 5 aa, Mus musculus
ATNEY

SEQ ID NO: 4 - protein, 6 aa, Mus musculus
ENVGTY

SEQ ID NO: 5 - protein, 9 aa, Mus musculus
GQSYSYPWT

SEQ ID NO: 6 - protein, 8 aa, Mus musculus
VNPNNGGT

SEQ ID NO: 7 - protein, 9 aa, Mus musculus
VPNWDWFAY

SEQ ID NO: 8 - protein, 11 aa, Mus musculus
QSLVHSNGNTY

SEQ ID NO: 9 - protein, 9 aa, Mus musculus
FQGSHVPFT

SEQ ID NO: 10 - protein, 9 aa, Mus musculus
APNWDWFAY

SEQ ID NO: 11 - protein, 11 aa, Mus musculus
QSILHSNGNTY

SEQ ID NO: 12 - protein, 112 aa, Mus musculus
EVQLQQSGPELVKPGSSVKISCKASGYTFTDYNMDWVKQSHGKSLEWIGAINPNNGGTSYNQKFKGKATLTVDKSSSTAYMELRSLTSEDSAVYYCATNEYWGQGTTLTVSS

SEQ ID NO: 13 - protein, 107 aa, Mus musculus
NIVMTQSPKSMSMSVGERVTLSCKASENVGTYVSWYQQKPEQSPKLLIYGASNRYTGVPDRFTGSGSATDFTLTISSVQAEDLADYHCGQSYSYPWTFGGGTKLEIK

SEQ ID NO: 14 - protein, 116 aa, Mus musculus
EVQLQQSGPELVKPGSSVKISCKASGYTFTDYNMVWVKQSHGKSLEWIGAVNPNNGGTSYNQKFKGKATLTVDKSSSTAYMELRSLTSEDSAVYYCVPNWDWFAYWGQGTLVTVSA

SEQ ID NO: 15 - protein, 112 aa, Mus musculus
DVLMTQTPLSLPVSLGDQASISCRSSQSLVHSNGNTYLEWYLQKPGQSPNLLIYKVSNRFSGVPDRFSGSGSGTDFTLKISRVEAEDLGVYYCFQGSHVPFTFGSGTKLEIK

SEQ ID NO: 16 - protein, 116 aa, Mus musculus
EVQLQQSGPELVKPGSSVKISCKPSGYTFTDYNMVWMKQSHGKSLEWIGAINPNNGGTTYNQKFKGKATLTVDKSSSTAYMELRSLTSEDSAVYYCAPNWDWFAYWGQGTLVTVSA

SEQ ID NO: 17 - protein, 112 aa, Mus musculus
DVLMTQTPLSLPVSLGDQASISCRSSQSILHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 18 - protein, 112 aa, Mus musculus
DVLMTQIPLSLPVSLGDQASISCRSSQSILHSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 19 - DNA, 336 bp, Mus musculus
gaggtccagctgcaacagtctggacctgagctggtgaagcctgggtcttcagtgaagatatcctgcaaagcttctggatacacattcactgactacaacatggactgggtgaagcagagccatggaaagagccttgagtggattggagctattaatcctaacaatggtggtactagctacaaccagaagttcaagggcaaggccacattgactgtagacaagtcctccagtacagcctacatggagctccgcagcctgacatctgaggactctgcagtctattactgtgcaacaaatgagtactggggccaaggcaccactctcacagtctcctca

SEQ ID NO: 20 - DNA, 321 bp, Mus musculus
aacattgtaatgacccaatctcccaaatccatgtccatgtcagtaggagagagggtcaccttgagctgcaaggccagtgagaatgtgggtacttatgtatcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagagttacagctatccgtggacgttcggtggaggcaccaagctggaaatcaaa

SEQ ID NO: 21 - DNA, 348 bp, Mus musculus
gaggtccagctgcaacagtctggacctgagctggtgaagcctgggtcttcagtgaagatatcctgcaaagcttctggatacacattcactgactacaacatggtctgggtgaagcagagccatggaaagagccttgagtggattggagctgttaatcctaacaatggtggtactagctacaaccagaagttcaagggcaaggccacattgactgtagacaagtcctccagcacagcctacatggagctccgcagcctgacatctgaggactctgcagtctattactgtgtccccaactgggactggtttgcttactggggccaagggactctggtcactgtctctgca

SEQ ID NO: 22 - DNA, 336 bp, Mus musculus
gatgttttgatgacccaaactccactctccctgcctgtcagtcttggagatcaagcctccatctcttgcagatctagtcagagccttgtacatagtaatggaaacacctatttagaatggtacctgcagaaaccaggccagtctccaaacctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaa

SEQ ID NO: 23 - DNA, 348 bp, Mus musculus
gaggtccagctgcaacagtctggacctgagctggtgaagcctgggtcttcagtgaagatatcctgcaaaccttctggatacacattcactgactacaacatggtctggatgaagcagagccatggaaagagccttgagtggattggagctattaatcctaacaatggtggtactacctacaaccagaagttcaagggcaaggccacattgactgtagacaagtcctccagcacagcctacatggagctccgcagcctgacatctgaggactctgcagtctattactgtgcccccaactgggactggtttgcttactggggccaagggactctggtcactgtctctgca

SEQ ID NO: 24 - DNA, 337 bp, Mus musculus
gatgttttgatgacccaaactccactctccctgcctgtcagtcttggagatcaagcctccatctcttgcagatctagtcagagcattttacatagtaatggaaacacctatttagaatggtacctgcagaaaccaggccagtctccaaagctcctgatctacaaagtttccaaccgattttccggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaac

SEQ ID NO: 25 - DNA, 337 bp, Mus musculus
gatgttttgatgacccaaattccactctccctgcctgtcagtcttggagatcaagcctccatctcttgcagatctagtcagagcattttacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaac

SEQ ID NO: 26 - protein, 431 aa, Homo sapiens
MEGVGGLWPWVLGLLSLPGVILGAPLASSCAGACGTSFPDGLTPEGTQASGDKDIPAINQGLILEETPESSFLIEGDIIRPSPFRLLSATSNKWPMGGSGVVEVPFLLSSKYDEPSRQVILEALAEFERSTCIRFVTYQDQRDFISIIPMYGCFSSVGRSGGMQVVSLAPTCLQKGRGIVLHELMHVLGFWHEHTRADRDRYIRVNWNEILPGFEINFIKSQSSNMLTPYDYSSVMHYGRLAFSRRGLPTITPLWAPSVHIGQRWNLSASDITRVLKLYGCSPSGPRPRGRGSHAHSTGRSPAPASLSLQRLLEALSAESRSPDPSGSSAGGQPVPAGPGESPHGWESPALKKLSAEASARQPQTLASSPRSRPGAGAPGVAQEQSWLAGVSTKPTVPSSEAGIQPVPVQGSPALPGGCVPRNHFKGMSED

SEQ ID NO: 27 - protein, 4 aa, Artificial Sequence
  A synthetic polypeptide
GGGS

SEQ ID NO: 28 - protein, 4 aa, Artificial Sequence
  A synthetic polypeptide
SGGG

SEQ ID NO: 29 - protein, 5 aa, Artificial Sequence
  A synthetic polypeptide
GGGGS

SEQ ID NO: 30 - protein, 5 aa, Artificial Sequence
  A synthetic polypeptide
SGGGG

SEQ ID NO: 31 - protein, 6 aa, Artificial Sequence
  A synthetic polypeptide
GGGGGS

SEQ ID NO: 32 - protein, 6 aa, Artificial Sequence
  A synthetic polypeptide
SGGGGG

SEQ ID NO: 33 - protein, 7 aa, Artificial Sequence
  A synthetic polypeptide
GGGGGGS

SEQ ID NO: 34 - protein, 7 aa, Artificial Sequence
  A synthetic polypeptide
SGGGGGG

SEQ ID NO: 35 - protein, 5 aa, Artificial Sequence
  A synthetic polypeptide
GGGGS

SEQ ID NO: 36 - protein, 5 aa, Artificial Sequence
  A synthetic polypeptide
SGGGG